SEQUENCE LISTING (40 sequences)

SEQ ID NO: 1 - protein, 10 aa, Artificial Sequence
  Synthetic polyleucine hydrophobic peptide.
LLLLLLLLLL

SEQ ID NO: 2 - protein, 15 aa, Artificial Sequence
  Synthetic 15 polyleucine hydrophobic peptide.
LLLLLLLLLLLLLLL

SEQ ID NO: 3 - protein, 10 aa, Artificial Sequence
  Synthetic valine hydrophobic peptide.
VVVVVVVVVV

SEQ ID NO: 4 - protein, 10 aa, Artificial Sequence
  Synthetic phenylalanine hydrophobic peptide.
FFFFFFFFFF

SEQ ID NO: 5 - protein, 6 aa, Artificial Sequence
  Synthetic hydrophilic polylysine and polyserine carrier or linker.
KKKKSS

SEQ ID NO: 6 - protein, 5 aa, Artificial Sequence
  Synthetic hydrophilic polylysine and serine carrier or linker.
SKKKK

SEQ ID NO: 7 - protein, 13 aa, Artificial Sequence
  Synthetic Pan-DR-helper cell epitope, PADRE.
AKFVAAWTLKAAA

SEQ ID NO: 8 - protein, 17 aa, Artificial Sequence
  Synthetic T helper epitope, P25.
KLIPNASLIENCTKAEL

SEQ ID NO: 9 - protein, 28 aa, Artificial Sequence
  J8, a synthetic epitope derived from amino acids 344-355 of the M protein of M1 GAS strain, flanked with GCN4 DNA-binding protein sequences.
QAEDKVKQSREAKKQVEKALKQLEDKVQ

SEQ ID NO: 10 - protein, 19 aa, group A streptococcus
EVLTRRQSQDPKYVTQRIS

SEQ ID NO: 11 - protein, 20 aa, group A streptococcus
DNGKAIYERARERALQELGP

SEQ ID NO: 12 - protein, 22 aa, Necator americanus
TSLIAGPKAQVEAIQKYIGAEL

SEQ ID NO: 13 - protein, 9 aa, Human papillomavirus type 16
RAHYNIVTF

SEQ ID NO: 14 - protein, 14 aa, Human papillomavirus type 16
QAEPDRAHYNIVTF

SEQ ID NO: 15 - protein, 17 aa, Severe acute respiratory syndrome coronavirus 2
AIHADQLTPTWRVYSTG

SEQ ID NO: 16 - protein, 40 aa, Severe acute respiratory syndrome coronavirus 2
STEIYQAGSTPCNGVEGFNCYFPLQSYGFQPTNGVGYQPY

SEQ ID NO: 17 - protein, 39 aa, Severe acute respiratory syndrome coronavirus 2
VGGNYNYLYRLFRKSNLKPFERDISTEIYQAGSTPCNGV

SEQ ID NO: 18 - protein, 14 aa, Severe acute respiratory syndrome coronavirus 2
FLPFQQFGRDIADT

SEQ ID NO: 19 - protein, 30 aa, Severe acute respiratory syndrome coronavirus 2
SVLYNSASFSTFKCYGVSPTKLNDLCFTNV

SEQ ID NO: 20 - protein, 42 aa, Artificial Sequence
  Synthetic peptide incorporating J8, lysine and PADRE.
QAEDKVKQSREAKKQVEKALKQLEDKVQKAKFVAAWTLKAAA

SEQ ID NO: 21 - protein, 41 aa, Artificial Sequence
  Synthetic peptide incorporating PADRE and J8.
AKFVAAWTLKAAAQAEDKVKQSREAKKQVEKALKQLEDKVQ

SEQ ID NO: 22 - protein, 32 aa, Artificial Sequence
  Synthetic peptide incorporating PADRE and PL1.
AKFVAAWTLKAAAEVLTRRQSQDPKYVTQRIS

SEQ ID NO: 23 - protein, 33 aa, Artificial Sequence
  Synthetic peptide incorporating PADRE and 88/30.
AKFVAAWTLKAAADNGKAIYERARERALQELGP

SEQ ID NO: 24 - protein, 40 aa, Artificial Sequence
  Synthetic peptide incorporating p3, lysine and P25.
TSLIAGPKAQVEAIQKYIGAELKKLIPNASLIENCTKAEL

SEQ ID NO: 25 - protein, 24 aa, Artificial Sequence
  Immunogenic agent, Compound Leu-8Qm, incorporating polyleucine hydrophobic peptide and 8Qm.
LLLLLLLLLLQAEPDRAHYNIVTF

SEQ ID NO: 26 - protein, 51 aa, Artificial Sequence
  Immunogenic agent incorporating polyleucine hydrophobic peptide, PADRE and J8.
LLLLLLLLLLAKFVAAWTLKAAAQAEDKVKQSREAKKQVEKALKQLEDKVQ

SEQ ID NO: 27 - protein, 42 aa, Artificial Sequence
  Immunogenic agent incorporating polyleucine hydrophobic peptide, PADRE and PL1.
LLLLLLLLLLAKFVAAWTLKAAAEVLTRRQSQDPKYVTQRIS

SEQ ID NO: 28 - protein, 43 aa, Artificial Sequence
  Immunogenic agent incorporating polyleucine hydrophobic peptide, PADRE and 88/30.
LLLLLLLLLLAKFVAAWTLKAAADNGKAIYERARERALQELGP

SEQ ID NO: 29 - protein, 57 aa, Artificial Sequence
  Immunogenic agent incorporating polyleucine hydrophobic peptide, PADRE, lysine and J8.
LLLLLLLLLLLLLLLAKFVAAWTLKAAAKQAEDKVKQSREAKKQVEKALKQLEDKVQ

SEQ ID NO: 30 - protein, 57 aa, Artificial Sequence
  Immunogenic agent incorporating polyleucine hydrophobic peptide, J8, lysine and PADRE.
LLLLLLLLLLLLLLLQAEDKVKQSREAKKQVEKALKQLEDKVQKAKFVAAWTLKAAA

SEQ ID NO: 31 - protein, 56 aa, Artificial Sequence
  Immunogenic agent incorporating 15 polyleucine hydrophobic peptide, PADRE and J8.
LLLLLLLLLLLLLLLAKFVAAWTLKAAAQAEDKVKQSREAKKQVEKALKQLEDKVQ

SEQ ID NO: 32 - protein, 56 aa, Artificial Sequence
  Immunogenic agent incorporating polyglutamine hydrophobic peptide, PADRE and J8.
QQQQQQQQQQQQQQQAKFVAAWTLKAAAQAEDKVKQSREAKKQVEKALKQLEDKVQ

SEQ ID NO: 33 - protein, 56 aa, Artificial Sequence
  Immunogenic agent incorporating polyglycine hydrophobic peptide, PADRE and J8.
GGGGGGGGGGGGGGGAKFVAAWTLKAAAQAEDKVKQSREAKKQVEKALKQLEDKVQ

SEQ ID NO: 34 - protein, 56 aa, Artificial Sequence
  Immunogenic agent incorporating polyserine hydrophobic peptide, PADRE and J8.
SSSSSSSSSSSSSSSAKFVAAWTLKAAAQAEDKVKQSREAKKQVEKALKQLEDKVQ

SEQ ID NO: 35 - protein, 56 aa, Artificial Sequence
  Immunogenic agent incorporating polyproline hydrophobic peptide, PADRE and J8.
PPPPPPPPPPPPPPPAKFVAAWTLKAAAQAEDKVKQSREAKKQVEKALKQLEDKVQ

SEQ ID NO: 36 - protein, 56 aa, Artificial Sequence
  Immunogenic agent incorporating alanine hydrophobic peptide, PADRE and J8.
AAAAAAAAAAAAAAAAKFVAAWTLKAAAQAEDKVKQSREAKKQVEKALKQLEDKVQ

SEQ ID NO: 37 - protein, 56 aa, Artificial Sequence
  Immunogenic agent incorporating phenylalanine- leucine-alanine repeat hydrophobic peptide, PADRE and J8.
FLAFLAFLAFLAFLAAKFVAAWTLKAAAQAEDKVKQSREAKKQVEKALKQLEDKVQ

SEQ ID NO: 38 - protein, 50 aa, Artificial Sequence
  Immunogenic agent, (Leu)10-B3, incorporating B3.
LLLLLLLLLLGVGGNYNYLYRLFRKSNLKPFERDISTEIYQAGSTPCNGV

SEQ ID NO: 39 - protein, 23 aa, Artificial Sequence
  Immunogenic agent, (Leu)10-PADRE, incorporating PADRE.
LLLLLLLLLLAKFVAAWTLKAAA

SEQ ID NO: 40 - protein, 20 aa, group A streptococcus
LRRDLDASREAKKQVEKALE

(19) United States
(12) Patent Application Publication
(10) Pub. No.: US 2023/0090311 A1
(43) Pub. Date: Mar. 23, 2023

(54) SELF-ASSEMBLING, SELF-ADJUVANTING SYSTEM FOR DELIVERY OF VACCINES

(71) Applicant: THE UNIVERSITY OF QUEENSLAND, St. Lucia, Queensland (AU)

(72) Inventors: Istvan TOTH, St. Lucia, Queensland (AU); Mariusz SKWARCZYNSKI, St. Lucia, Queensland (AU); Guangzu ZHAO, St. Lucia, Queensland (AU)

(21) Appl. No.: 17/791,856

(22) PCT Filed: Jan. 8, 2021
(86) PCT No.: PCT/AU2021/050012

§ 371 Date: Jul. 8, 2022

(30) Foreign Application Priority Data
Jan. 9, 2020 (AU) 2020900058

(51) Int. Cl. A61K 39/215; A61P 31/14

(52) U.S. Cl. CPC A61K 39/215; A61P 31/14; A61K 2039/6031

(57) ABSTRACT

This invention relates to an immunogenic agent comprising a hydrophobic peptide covalently coupled or conjugated to at least one antigenic molecule that facilitates self-assembly of a plurality of the immunogenic agents into an immunogenic complex that has adjuvant properties. The invention also relates to a method of eliciting an immune response in a subject, comprising the step of administering to the subject at least one immunogenic agent or at least one immunogenic complex to thereby elicit an immune response in the subject.

### RELATED APPLICATIONS

This application claims priority of Australian Provisional Patent Application No. 2020900058, filed 9 Jan. 2020, the entire contents of which are incorporated herein by reference.

### TECHNICAL FIELD

THIS INVENTION relates to immunogenic agents. More particularly, this invention relates to an immunogenic agent comprising a hydrophobic peptide covalently coupled or conjugated to at least one antigenic molecule that facilitates self-assembly of a plurality of the immunogenic agents into an immunogenic complex that has adjuvant properties.

### BACKGROUND

Peptide- and protein-based vaccines for their optimal effectiveness need the help of safe but powerful adjuvants. Many currently available adjuvants are too weak to stimulate potent immune responses against peptides and proteins. In contrast, bacteria-derived and oil-based adjuvants are typically strong enough to trigger immune responses against peptides but can cause undesirable reactions, including allergic responses and excessive inflammation. Self-assembling and self-adjuvanting delivery systems based on polymers have recently demonstrated high efficiency in stimulation of immune responses against carried peptide antigens without generating adverse effects. Unfortunately, such systems are usually not biodegradable and their chemical compositions as well as stereochemistry are never fully defined. Accordingly, there remains a need for delivery or adjuvant systems for peptide- and protein-based vaccines that overcome one or more of the deficiencies of such prior art delivery systems.

### DETAILED DESCRIPTION

To at least partly address and overcome one or more disadvantages of the prior art, such as discussed above, the present invention in one or more embodiments provides a peptide-based vaccine delivery system based on fully-defined and biodegradable polymers built from hydrophobic amino acids (HAA).

The present invention in one or more embodiments broadly provides an immunogenic agent that can self-assemble into an immunogenic complex for delivery to a subject and elicit an immune response. Suitably, the immunogenic complex has “self-adjuvanting” properties that obviate the need to co-administer an adjuvant or other general immune-stimulant.

The present invention, in one or more embodiments, broadly provides an immunogenic agent that can self-assemble into an immunogenic complex for delivery to a subject and elicit an immune response. Suitably, the immunogenic complex has “self-adjuvanting” properties that obviates the need to co-administer an adjuvant or other general immune-stimulant.

According to a first aspect of the present invention, there is provided an immunogenic agent suitable for administration to a subject, said immunogenic agent comprising a hydrophobic peptide covalently coupled or conjugated to at least one antigenic molecule.

According to a second aspect of the present invention, there is provided an immunogenic agent having the structure: [Antigenic molecule]ₙ-[Carrier/Linker]ₘ-[Hydrophobic peptide]

wherein: n is 1, 2, 3 or a higher number; m is 0, 1, 2, 3 or a higher number; and “-” represents a covalent coupling or conjugation.

According to a third aspect of the present invention, there is provided a method of producing an immunogenic agent, comprising the step of covalently coupling or conjugating a hydrophobic peptide to at least one antigenic molecule to thereby produce the immunogenic agent.

According to a fourth aspect of the present invention, there is provided an immunogenic agent when produced by the method according to the third aspect.

According to a fifth aspect of the present invention, there is provided an immunogenic complex comprising a plurality of immunogenic agents according to the first, second or fourth aspects, wherein a plurality of hydrophobic peptides interact in the immunogenic complex.

According to a sixth aspect of the present invention, there is provided a method of producing an immunogenic complex comprising the step of combining a plurality of immunogenic agents of the first, second or fourth aspects, whereby the plurality of immunogenic agents self-assemble into the immunogenic complex.

According to a seventh aspect of the present invention, there is provided an immunogenic complex when produced by the method of the sixth aspect.

According to an eighth aspect of the present invention, there is provided a composition comprising at least one said immunogenic agent of any one of the first, second and fourth aspects, and/or at least one immunogenic complex of the fifth or seventh aspects.

According to a ninth aspect of the present invention, there is provided a method of eliciting an immune response in a subject, said method comprising the step of administering to the subject at least one said immunogenic agent of any one of the first, second and fourth aspects, at least one said immunogenic complex of the fifth or seventh aspects, or at least one said composition of the eighth aspect, to thereby elicit an immune response in the subject.

According to a tenth aspect of the present invention, there is provided a method of immunizing a subject, said method comprising the step of administering to the subject at least one said immunogenic agent of any one of the first, second and fourth aspects, at least one said immunogenic complex of the fifth or seventh aspects, or at least one said composition of the eighth aspect, to thereby immunize the subject.

According to an eleventh aspect of the present invention, there is provided a method of treating or preventing a disease, disorder or condition in a subject, said method comprising the step of administering to the subject at least one said immunogenic agent of any one of the first, second and fourth aspects, at least one said immunogenic complex of the fifth or seventh aspects, or at least one said composition of the eighth aspect.

According to a twelfth aspect of the present invention, there is provided use of at least one said immunogenic agent of any one of the first, second and fourth aspects, at least one said immunogenic complex of the fifth or seventh aspects, or at least one said composition of the eighth aspect, in the preparation of a medicament for eliciting an immune response in a subject.

According to a thirteenth aspect of the present invention, there is provided use of at least one said immunogenic agent of any one of the first, second and fourth aspects, at least one said immunogenic complex of the fifth or seventh aspects, or at least one said composition of the eighth aspect, in the preparation of a medicament for immunizing a subject.

According to a fourteenth aspect of the present invention, there is provided use of at least one said immunogenic agent of any one of the first, second and fourth aspects, at least one said immunogenic complex of the fifth or seventh aspects, or at least one said composition of the eighth aspect, in the preparation of a medicament for treating or preventing a disease, disorder or condition in a subject.

According to a fifteenth aspect of the present invention, there is provided at least one said immunogenic agent of any one of the first, second and fourth aspects, at least one said immunogenic complex of the fifth or seventh aspects, or at least one said composition of the eighth aspect, for use in eliciting an immune response in the subject.

According to a sixteenth aspect of the present invention, there is provided at least one said immunogenic agent of any one of the first, second and fourth aspects, at least one said immunogenic complex of the fifth or seventh aspects, or at least one said composition of the eighth aspect, for use in immunizing a subject.

According to a seventeenth aspect of the present invention, there is provided at least one said immunogenic agent of any one of the first, second and fourth aspects, at least one said immunogenic complex of the fifth or seventh aspects, or at least one said composition of the eighth aspect, for use in treating or preventing a disease, disorder or condition in a subject.

Preferably the immunogenic agent is a single molecular entity. Preferably the immunogenic agent is amphiphilic.

In some embodiments, the immunogenic complex is capable of inducing production of opsonic epitope-specific antibodies without the help of any external adjuvant. In some embodiments, the immunogenic complex acts as a strong humoral adjuvant.

The hydrophobic peptide can be of any suitable length and can consist of any suitable number and type or types of amino acid residues. The hydrophobic peptide can comprise a plurality of hydrophobic natural, non-natural, D- and/or L-amino acids. In some embodiments, the hydrophobic peptide comprises a plurality of hydrophobic natural amino acids.

In some embodiments, the hydrophobic peptide comprises, consists essentially of, or consists of anywhere between 2 and about 50 amino acids, including all integers between 2 and 50, including 2, 3, 4, 5 etc. In some embodiments, the hydrophobic peptide comprises at least two (2), three (3), four (4), five (5), six (6), seven (7), eight (8), nine (9), ten (10), eleven (11), twelve (12), thirteen (13), fourteen (14), fifteen (15), or any range therein, contiguous, hydrophobic amino acids. By way of example, the hydrophobic peptide may comprise 10 to 15 contiguous amino acids. In some embodiments, the hydrophobic peptide comprises no more than about fifty (50), forty (40), thirty (30), twenty-five (25), twenty (20), nineteen (19), eighteen (18), seventeen (17), sixteen (16), or fifteen (15) contiguous, hydrophobic amino acids. In some embodiments, the hydrophobic peptide comprises, consists essentially of, or consists of anywhere between 2-30, 3-25, 4-20, 5-20, 7-18, 5-15 or 10-15 contiguous, hydrophobic amino acids. In some embodiments, the hydrophobic peptide comprises, consists essentially of, or consists of 10 or 15 amino acids.

In some embodiments, the hydrophobic peptide consists of the same amino acid. In some embodiments, the hydrophobic peptide consists of two, three or more different amino acids (eg. phenylalanine-leucine-alanine). In some embodiments, the hydrophobic amino acids can include alanine, proline, glycine, tyrosine, valine, leucine, isoleucine, tryptophan, phenylalanine and/or methionine. In some embodiments, the hydrophobic amino acids can be selected from the group consisting of: glycine, proline, valine, alanine, phenylalanine and leucine. In some embodiments, the hydrophobic peptide consists of 10 valine residues, 10 phenylalanine residues, 10 leucine residues, 15 leucine residues, 15 glycine residues, 15 proline residues, 15 alanine residues, 25 alanine residues, or 5 phenylalanine-leucine-alanine repeats.

In some embodiments, the hydrophobic peptide enables anchoring of the immunogenic agent to a liposomal membrane, or other lipophilic surface such as a micelle or nanoparticle. As natural protein transmembrane domains are often leucine rich, in embodiments where the hydrophobic peptide consists of or comprises polyleucine, the hydrophobic peptide may allow anchoring of the immunogenic agent to a liposomal membrane.

Although not wishing to be bound by theory, the length and particular hydrophobic amino acid composition of the hydrophobic peptide may be optimized in light of the particular at least one antigenic molecule of the immunogenic agent. By way of example, hydrophilic antigenic molecules will usually require a longer hydrophobic peptide, whereas antigenic molecules that are not as hydrophilic (e.g., having some degree of hydrophobicity) may require a shorter hydrophobic peptide. Such optimization will minimize or eliminate the risk of precipitation. In some embodiments, the hydrophobic peptide can induce the formation of alpha-helices and/or beta-sheets.

In some embodiments, the conformation of the at least one antigenic molecule (epitope) is controlled by changing the amino acid sequence and/or length of the hydrophobic peptide.

In some embodiments, the solubility of the immunogenic agent is controlled by changing the amino acid sequence and/or length of the hydrophobic peptide.

In some embodiments, the immunogenic agent can further comprise at least one carrier or linker to which the hydrophobic peptide and at least one antigenic molecule are directly or indirectly covalently coupled or conjugated. The immunogenic agent can comprise 1, 2, 3, 4, 5 or more carriers or linkers. These can be the same or different from each other.

In some embodiments, the immunogenic agent can be essentially linear. In some embodiments, the immunogenic agent can be branched or dendritic in structure. In embodiments with a plurality of antigenic molecules, the carrier or linker may be utilised to couple or connect the antigenic molecules together in a contiguous or branched form or manner.

In some embodiments the at least one carrier or linker functions as a spacer. In some embodiments the at least one carrier or linker can balance the hydrophobic:hydrophilic ratio of the immunogenic agent to assist with proper conformation of the immunogenic agent/at least one antigenic molecule, and/or solubility of the immunogenic agent. In some embodiments, the carrier or linker can induce the formation of alpha-helices and/or beta-sheets.

In some embodiments the at least one carrier or linker can provide the immunogenic agent with a linear structure. As mentioned, in some embodiments, the at least one carrier or linker can provide the immunogenic agent with a branched or dendritic structure.

The at least one carrier or linker can comprise one or more of the following: an amino acid, a peptide, a carbohydrate and/or a polymeric molecule. In some embodiments, the at least one carrier or linker can comprise one or a plurality of same or different amino acids or other suitable molecules such as described above.

In some embodiments, the at least one carrier or linker comprises at least one amino acid that is capable of forming a branched chain, for attachment to the hydrophobic peptide. Any suitable amino acid, such as lysine or cysteine, can be used. In some embodiments, the at least one carrier or linker amino acid or amino acids comprise one or more free amino groups. Suitably, the amino acid carrier or linker comprises at least two (2) free amino groups. In a particular form of this embodiment the amino acid is a single lysine residue or a branched chain polylysine with or without one or more serine residues. In some embodiments, the at least one carrier or linker amino acid or amino acids comprise one or more cysteine residues having one or more free thiol groups. Suitably, the amino acid carrier or linker comprises one cysteine residue having a free thiol group. In another embodiment, the at least one carrier or linker comprises one or a plurality of acidic amino acids, such as aspartic acid and glutamic acid, although without limitation thereto.

In some embodiments, the at least one carrier or linker comprises the peptide lysine-lysine-lysine-lysine-serine-serine (SEQ ID NO:5). In some embodiments, the at least one carrier or linker comprises the peptide serine-lysine-lysine-lysine-lysine (SEQ ID NO:6). In some embodiments, the at least one carrier or linker comprises a polyol or polyether, such as polyethylene glycol (PEG).

In some embodiments, any or all N-terminal ends of the immunogenic agent can be acetylated (eg. the at least one antigenic molecule and/or hydrophobic peptide). Likewise, any or all C-terminal ends of the immunogenic agent can be subjected to amidation (eg. the linker). These modifications can reduce the total charge and solubility of the immunogenic agent.

The at least one antigenic molecule may be any molecule capable of eliciting an immune response upon administration to a subject, at least when present in the immunogenic agent. By way of example, the antigenic molecule may be a protein, peptide, carbohydrate, lipid or combination of these such as a glycoprotein, proteoglycan, lipoprotein, glycolipoprotein or fragment, variant or derivative thereof.

Any suitable number of antigenic molecules can be used. This includes 1, 2, 3, 4, 5, 6, 7, 8, 9, 10 or even more antigenic molecules. If two or more antigenic molecules are used, then these can be the same as each other or different from each other. The antigenic molecule can be hydrophilic or hydrophobic.

Generally, a fragment may be a portion, segment, subunit, moiety, monomer, sub-sequence, region or domain of the antigenic molecule. In the specific context of a protein, a “fragment” is a segment, domain, portion or region of a protein, which constitutes less than 100% of the amino acid sequence of the antigenic molecule protein. It will be appreciated that the fragment may be a single fragment or may be repeated alone or with other fragments. Peptide fragments of antigenic molecules may comprise, consist essentially of or consist of at least 5, 6, 7, 8, 9, 10, 11, 12, 13, 14, 15, 16, 17, 18, 19, 20, 21, 22, 25, 30, 35 or more contiguous amino acids.

In some embodiments, the at least one antigenic molecule comprises, consists essentially of, or consists of a peptide or a protein. In some embodiments, the at least one antigenic molecule is or comprises a peptide antigen comprising at least one epitope.

In some embodiments, the antigenic molecule is, or comprises, a synthetic epitope that is not derived from, or directed to, a particular pathogen. Such synthetic epitopes may be “universal” epitopes that elicit a broad spectrum immune response (such as a helper T cell or memory T cell response) when coupled with, or administered with, an epitope from a particular pathogen. A non-limiting example is a non-natural Pan-DR-helper cell epitope (PADRE; amino acid sequence: AKFVAAWTLKAAA; SEQ ID NO:7) or P25 (amino acid sequence: KLIPNASLIENCTKAEL; SEQ ID NO:8), such as described hereinafter.

In some embodiments, the at least one antigenic molecule consists of or comprises a B-cell epitope. In some embodiments, the at least one antigenic molecule consists of or comprises a T-cell (memory or helper) epitope. In some embodiments the at least one antigenic molecule comprises both a B cell epitope and a T cell epitope.

As generally used herein, an “epitope” is an antigenic or preferably immunogenic fragment, such as of a peptide, protein or carbohydrate or other molecule, or combination of these, such as a glycoprotein or proteoglycan. It will be appreciated that the epitope may be continuous or discontinuous, the latter being particularly relevant to B-cell epitopes.

In one embodiment, the at least one antigenic molecule, or fragment thereof, is of a pathogen, or of a molecular component of the pathogen. As generally used herein, when a molecule (such as a protein) is referred to as being “of” or “from” a pathogen is meant that the molecule is at least partly, or entirely, present in or derived from a pathogen.

Pathogens may include viruses, bacteria, fungi, yeasts, protists, worms and/or any other organism capable of causing a disease, disorder or condition in a subject. By way of example only, these pathogens may include: bacteria of genera such as Staphylococcus, Bacillus, Yersinia, Henophilus, Streptococcus, Neisseria, Klebsiella, Brucella, Bordatella, Clostridium, Listeria. Legionella, Vibrio, Salmonella and Shigella, although without limitation thereto; viruses such as coronavirus (eg. SARS-CoV-2), influenza virus, rhinovirus, Epstein Barr virus, varicella zoster, mumps virus, measles virus, cytomegalovirus, human immunodeficiency virus, papillomavirus, poliovirus, hepatitis virus, H-endra virus, flaviviruses and herpesviruses, although without limitation thereto: protists such as Plasmodium (causing malaria), Leishmania, Giardia and Babesia parasites; and worms such as schistosomes, trematodes, nematodes (e.g., hookworms) and other helminths, although without limitation thereto.

In some embodiments, the at least one antigenic molecule consists of or comprises a peptide derived from Group A Streptococcus (GAS) major virulent factor M-protein. In some embodiments, the at least one antigenic molecule consists of or comprises a peptide derived from the C repeat region of the M-protein. In some embodiments, the at least one antigenic molecule consists of or comprises a J8 peptide (QAEDKVKQSREAKKQVEKALKQLEDKVQ; SEQ ID NO:9) derived from Group A Streptococcus (GAS) major virulent factor M-protein. J8 derives from amino acids 344-355 of the M protein of M1 GAS strain, flanked with GCN4 DNA-binding protein sequences.

In some embodiments, the at least one antigenic molecule consists of or comprises the PL1 B-cell epitope (EVLTRRQSQDPKYVTQRIS; SEQ ID NO:10) derived from the N-terminal of M protein of GAS strain M54.

In some embodiments, the at least one antigenic molecule consists of or comprises the 88/30 B-cell epitope (DNGKAIYERARERALQELGP; SEQ ID NO:11) derived from the N-terminal of M protein of GAS strain 88/30.

In some embodiments, the immunogenic agent comprises a hydrophobic 10 to 15 amino acid peptide covalently coupled or conjugated to the GAS B-cell epitope J8 (SEQ ID NO:9) and the universal T-helper epitope PADRE (SEQ ID NO:7).

In some embodiments, the immunogenic agent comprises a hydrophobic 10 amino acid peptide, preferably consisting of polyleucine, covalently coupled or conjugated to the GAS B-cell epitope J8 (SEQ ID NO:9) and the universal T-helper epitope PADRE (SEQ ID NO:7).

In some embodiments, the immunogenic agent comprises a hydrophobic 15 amino acid peptide, preferably consisting of polyleucine, covalently coupled or conjugated to the universal T-helper epitope PADRE (SEQ ID NO:7) which, in turn, is coupled or conjugated to the GAS B-cell epitope J8 (SEQ ID NO:9).

In some embodiments, the immunogenic agent has the structure of Compound 5 of FIG. 1.

In some embodiments, the immunogenic agent has the structure of Compound 7 of FIG. 22 b) (SEQ ID NO:26).

In some embodiments, the immunogenic agent comprises a hydrophobic 10 to 15 amino acid peptide covalently coupled or conjugated to the GAS B-cell epitope PL1 (SEQ ID NO:10) and the universal T-helper epitope PADRE (SEQ ID NO:7).

In some embodiments, the immunogenic agent has the structure of Compound 8 of FIG. 22 b) (SEQ ID NO:27).

In some embodiments, the immunogenic agent comprises a hydrophobic 10 to 15 amino acid peptide covalently coupled or conjugated to the GAS B-cell epitope 88/30 (SEQ ID NO:11) and the universal T-helper epitope PADRE (SEQ ID NO:7).

In some embodiments, the immunogenic agent has the structure of Compound 9 of FIG. 22 b) (SEQ ID NO:28).

In some embodiments, the immunogenic agent comprises a hydrophobic 10 to 15 amino acid peptide covalently coupled or conjugated to the GAS B-cell epitopes J8 (SEQ ID NO:9), PL1 (SEQ ID NO:10) and 88/30 (SEQ ID NO:11), and the universal T-helper epitope PADRE (SEQ ID NO:7).

In some embodiments, the immunogenic agent has the structure of Compound 14 of FIG. 22 b).

In some embodiments, Compounds 7, 8 and 9 of FIG. 22 b) are combined to form the immunogenic complex.

In some embodiments, the immunogenic agent has the structure of one of the following:

Ac-J8-K(Ac-(Leu)₁₅)PADRE, wherein “Ac” denotes acetylation and “K” denotes lysine;

Ac-(Leu)₁₅-PADRE-K-J8, wherein “Ac” denotes acetylation and “K” denotes lysine (SEQ ID NO:29);

Ac-(Leu)₁₅-J8-K-PADRE, wherein “Ac” denotes acetylation and “K” denotes lysine (SEQ ID NO:30);

Ac-(Leu)₁₅-PADRE-J8, wherein “Ac” denotes acetylation (SEQ ID NO:31);

Ac-(Glu)₁₅-PADRE-J8, wherein “Ac” denotes acetylation (SEQ ID NO:32);

Ac-(Gly)₁₅-PADRE-J8, wherein “Ac” denotes acetylation (SEQ ID NO:33);

Ac-(Ser)₁₅-PADRE-J8, wherein “Ac” denotes acetylation (SEQ ID NO:34);

Ac-(Pro)₁₅-PADRE-J8, wherein “Ac” denotes acetylation (SEQ ID NO:35);

Ac-(Ala)₁₅-PADRE-J8, wherein “Ac” denotes acetylation (SEQ ID NO:36); or

Ac-(Phe-Leu-Ala)₅-PADRE-J8, wherein “Ac” denotes acetylation (SEQ ID NO:37).

In some embodiments, the immunogenic agent has the structure of any one of Compounds 2 and 3 to 11 of FIG. 26 (SEQ ID NOs:29-37).

In some embodiments, the at least one antigenic molecule consists of or comprises a peptide derived from a spike protein of a coronavirus. In some embodiments, the at least one antigenic molecule consists of or comprises a peptide derived from SARS-CoV-2 spike protein. In some embodiments, the at least one antigenic molecule consists of or comprises: ACE2-RBD critical binding amino acid residues; amino acid residues at or near S1/S2 cleavage/priming site; amino acid residues at receptor binding motif of RBD; or amino acid residues at NTD of receptor binding domain but not in direct contact with ACE2.

In some embodiments, the at least one antigenic molecule consists of or comprises the SARS-CoV spike protein epitope AIHADQLTPTWRVYSTG (S₆₂₃₋₆₃₉) (SEQ ID NO:15).

In some embodiments, the at least one antigenic molecule consists of or comprises the SARS-CoV spike protein epitope STEIYQAGSTPCNGVEGFNCYFPLQSYGFQPTNGVGYQPY (S₄₆₉₋₅₀₈) (SEQ ID NO:16).

In some embodiments, the at least one antigenic molecule consists of or comprises the SARS-CoV spike protein epitope VGGNYNYLYRLFRKSNLKPFERDISTEIYQAGSTPCNGV (S₄₄₅₋₄₈₃) (SEQ ID NO:17).

In some embodiments, the at least one antigenic molecule consists of or comprises the SARS-CoV spike protein epitope FLPFQQFGRDIADT (S₅₅₉₋₅₇₂) (SEQ ID NO:18).

In some embodiments, the at least one antigenic molecule consists of or comprises the SARS-CoV spike protein epitope SVLYNSASFSTFKCYGVSPTKLNDLCFTNV (S₃₆₆₋₃₉₅) (SEQ ID NO:19).

In some embodiments, the immunogenic agent comprises a hydrophobic 10 to 15 amino acid peptide covalently coupled or conjugated to an SARS-CoV spike protein epitope, preferably an epitope as described herein.

In some embodiments, the immunogenic agent comprises a hydrophobic 10 to 15 amino acid peptide covalently coupled or conjugated to an SARS-CoV spike protein epitope and the T-helper epitope PADRE (SEQ ID NO:7).

In some embodiments, the immunogenic agent comprises a hydrophobic 10 to 15 amino acid peptide covalently coupled or conjugated to an SARS-CoV spike protein epitope and at least one carrier or linker, such as a peptide or protein.

In some embodiments, the immunogenic agent comprises a hydrophobic 10 to 15 amino acid peptide covalently coupled or conjugated to VGGNYNYLYRLFRKSNLKPFERDISTEIYQAGSTPCNGV (SEQ ID NO:17). In some embodiments the hydrophobic peptide consists of leucine, valine, phenylalanine, glycine, proline, alanine, phenylalanine-leucine-alanine tri-peptide repeat residues, or any combination of these.

In some embodiments, the immunogenic agent is LLLLLLLLLLVGGNYNYLYRLFRKSNLKPFERDISTEIYQAGSTPCNGV (Compound (Leu)₁₀-B3; SEQ ID NO:38).

In some embodiments, the immunogenic agent comprises a hydrophobic 10 to 15 amino acid peptide covalently coupled or conjugated to the universal T-helper epitope PADRE (SEQ ID NO:7). In some embodiments, the immunogenic agent comprises a hydrophobic 10 leucine amino acid peptide covalently coupled or conjugated to the universal T-helper epitope PADRE (LLLLLLLLLLAKFVAAWTLKAAA; Compound (Leu)₁₀-PADRE; SEQ ID NO:39).

In some embodiments, Compounds (Leu)₁₀-B3 (SEQ ID NO:38) and (Leu)₁₀-PADRE (SEQ ID NO:39) are combined to form the immunogenic complex.

In some embodiments, the immunogenic agent or immunogenic complex can be delivered in a composition comprising liposomes (described later in this specification).

In some embodiments, the immunogenic agent or immunogenic complex can be anchored into the liposomes by way of the hydrophobic peptide.

In some embodiments, the immunogenic agent or immunogenic complex can be delivered in a composition comprising liposomes and a mannose targeting moiety incorporated into the liposomes (described later in this specification).

In yet another embodiment, the at least one antigenic molecule is, or comprises, a tumour antigen or fragment thereof. As used herein, a “tumour antigen” may be a protein, glycoprotein (or a carbohydrate-containing fragment), lipoprotein or other molecule expressed by tumour cells. This may include “unique” expression by tumour cells (e.g., as a result of viral infection such as by HPV (Human Papillomavirus)), aberrant expression of endogenous molecules compared to normal or non-tumour cells such as by expression of a molecule not normally expressed by that cell type, over-expression of the endogenous molecule by tumour cells and/or expression of a modified or mutated molecule by the tumour cells (e.g., due to mutated oncogenes or tumour suppressor genes).

In some embodiments, the at least one antigenic molecule consists of or comprises a peptide derived from an oncoprotein. In some embodiments, the at least one antigenic molecule consists of or comprises a peptide derived from a HPV oncoprotein. In some embodiments, the at least one antigenic molecule consists of or comprises a peptide derived from the HPV oncoprotein called E7. In some embodiments, the at least one antigenic molecule consists of or comprises a CD8+ peptide, which can activate cytotoxic T lymphocytes, which can in turn destroy tumour cells. In some embodiments, the at least one antigenic molecule consists of or comprises 8Qm (E7_{44_57}, QAEPDRAHYNIVTF; SEQ ID NO:14), derived from HPV-16 E7 oncoprotein which bears both a CTL epitope (CD8+ CTL) and a T helper (CD4+) epitope in the single sequence.

In some embodiments, the immunogenic agent comprises a hydrophobic 10 to 15 amino acid leucine polypeptide covalently coupled or conjugated to 8Qm (SEQ ID NO:14).

In some embodiments, the immunogenic agent has the structure of Compound Leu-8Qm of FIG. 14 b) (LLLLLLLLLLQAEPDRAHYNIVTF; SEQ ID NO:25).

In some embodiments, the immunogenic agent or immunogenic complex can be delivered in a composition comprising liposomes (described later in this specification). In some embodiments, the immunogenic agent or immunogenic complex can be anchored into the liposomes by way of the hydrophobic peptide. In some embodiments, the immunogenic agent or immunogenic complex can be delivered in a composition comprising liposomes and a mannose targeting moiety incorporated into the liposomes (described later in this specification).

In some embodiments, the at least one antigenic molecule consists of or comprises a peptide derived from a hookworm. In some embodiments, the at least one antigenic molecule consists of or comprises a Necator americanus peptide derived from Na-APR-1, called A₂₉₁Y. In some embodiments, the at least one antigenic molecule consists of or comprises a B cell epitope called p3 (TSLIAGPKAQVEAIQKYIGAEL; SEQ ID NO:12).

As mentioned, in some embodiments, the antigenic molecule is, or comprises, a synthetic epitope that is not derived from, or directed to, a particular pathogen.

In some embodiments, the immunogenic agent comprises a hydrophobic 10 to 15 amino acid peptide covalently coupled or conjugated to the epitope p3 (SEQ ID NO:12) and the universal T-helper epitope P25 (SEQ ID NO:8).

In some embodiments, the immunogenic agent comprises a hydrophobic 10 amino acid peptide, preferably consisting of polyleucine, covalently coupled or conjugated to the epitope p3 (SEQ ID NO:12) and the universal T-helper epitope P25 (SEQ ID NO:8). In some embodiments, the immunogenic agent further comprises at least one carrier or linker, preferably a lysine branching spacer attached to the hydrophobic peptide, and/or a hydrophilic moiety built from lysine and serine to increase the solubility and allow for self-assembly of the epitopes.

In some embodiments, the immunogenic agent has the structure of Compound 3 of FIG. 18.

In some embodiments, the immunogenic agent or immunogenic complex is suitable for oral administration to the subject. In this case, the composition can comprise a liquid excipient or carrier containing said immunogenic agent and/or immunogenic complex.

It will also be appreciated that the invention provides an immunogenic complex that may comprise a plurality of the same and/or different immunogenic agents disclosed herein. Thus, immunogenic complexes may comprise a plurality of immunogenic agents that each have the same B- and/or T-cell epitopes, or may comprise a heterogeneous mixture of immunogenic agents wherein there are a plurality of different T and/or B epitopes in the immunogenic complex. It will be understood that the different T and/or B epitopes in the immunogenic complex could be different epitopes from the same pathogen or could be different epitopes from different pathogens, the latter providing a multivalent immunogenic complex that could be used to immunize against a plurality of different pathogens.

It will also be appreciated that epitopes may be artificially modified, or where natural allelic variation results in variation in an epitope between individuals of a species or between species. By way of example, this may be relevant to peptide epitopes and non-peptide epitopes such as carbohydrate epitopes.

The at least one antigenic molecule can be a variant of any one of the protein, peptide or polypeptide sequences described herein, including any one of the relevant sequences listed in the Sequence Listing (eg. J8) or as shown in the Figures. As specifically used herein, a protein or peptide “variant” shares a definable amino acid sequence relationship with a reference amino acid sequence. The “variant” protein may have one or a plurality of amino acids of the reference amino acid sequence deleted or substituted by different amino acids. It is well understood in the art that some amino acids may be substituted or deleted without adversely affecting the immunogenicity of the immunogenic fragment (conservative substitutions). In some embodiments, modification of the amino acid sequence may improve immunogenicity. Preferably, protein or peptide variants share at least 70% or 75%, preferably at least 80% or 85% or more preferably at least 90%, 91%, 92%, 93%, 94%, 95%, 96%, 97%, 98% or 99% sequence identity with a reference amino acid sequence.

Terms used generally herein to describe sequence relationships between respective proteins and nucleic acids include “comparison window”, “sequence identity”, “percentage of sequence identity” and “substantial identity”. Because respective nucleic acids/proteins may each comprise (1) only one or more portions of a complete nucleic acid/protein sequence that are shared by the nucleic acids/proteins, and (2) one or more portions which are divergent between the nucleic acids/proteins, sequence comparisons are typically performed by comparing sequences over a “comparison window” to identify and compare local regions of sequence similarity. A “comparison window” refers to a conceptual segment of typically 6, 9 or 12 contiguous residues that is compared to a reference sequence. The comparison window may comprise additions or deletions (i.e., gaps) of about 20% or less as compared to the reference sequence for optimal alignment of the respective sequences. Optimal alignment of sequences for aligning a comparison window may be conducted by computerised implementations of algorithms (Geneworks program by Intelligenetics; GAP, BESTFIT, FASTA, and TFASTA in the Wisconsin Genetics Software Package Release 7.0, Genetics Computer Group, 575 Science Drive Madison, Wis., USA, incorporated herein by reference) or by inspection and the best alignment (i.e. resulting in the highest percentage homology over the comparison window) generated by any of the various methods selected. Reference also may be made to the BLAST family of programs as for example disclosed by Altschul et al., 1997, Nucl. Acids Res. 25 3389, which is incorporated herein by reference. A detailed discussion of sequence analysis can be found in Unit 19.3 of CURRENT PROTOCOLS IN MOLECULAR BIOLOGY Eds. Ausubel et al. (John Wiley & Sons Inc NY, 1995-1999).

The term “sequence identity” is used herein in its broadest sense to include the number of exact nucleotide or amino acid matches having regard to an appropriate alignment using a standard algorithm, having regard to the extent that sequences are identical over a window of comparison. Thus, a “percentage of sequence identity” is calculated by comparing two optimally aligned sequences over the window of comparison, determining the number of positions at which the identical nucleic acid base (e.g., A, T, C, G, I) occurs in both sequences to yield the number of matched positions, dividing the number of matched positions by the total number of positions in the window of comparison (i.e., the window size), and multiplying the result by 100 to yield the percentage of sequence identity. For example, “sequence identity” may be understood to mean the “match percentage” calculated by the DNASIS computer program (Version 2.5 for windows; available from Hitachi Software engineering Co., Ltd., South San Francisco, Calif., USA).

As used herein, “derivatives” are molecules such as proteins, fragments or variants thereof that have been altered, for example by conjugation or complexing with other chemical moieties, by post-translational modification (e.g. phosphorylation, acetylation, amidation and the like), modification of glycosylation (e.g. adding, removing or altering glycosylation), lipidation and/or inclusion of additional amino acid sequences as would be understood in the art.

Additional amino acid sequences may include fusion partner amino acid sequences which create a fusion protein. By way of example, fusion partner amino acid sequences may assist in detection and/or purification of the isolated fusion protein. Non-limiting examples include metal-binding (e.g. polyhistidine) fusion partners, maltose binding protein (MBP), Protein A, glutathione S-transferase (GST), fluorescent protein sequences (e.g. GFP), epitope tags such as myc, FLAG and haemagglutinin tags.

Other derivatives contemplated by the invention include, but are not limited to, modification to side chains, incorporation of unnatural amino acids and/or their derivatives during peptide, or protein synthesis and the use of crosslinkers and other methods which impose conformational constraints on the immunogenic proteins, fragments and variants of the invention. Peptides may also be flanked with specific peptide sequences, such as to maintain its helicity, an example being J8 peptide (SEQ ID NO:9) that includes a GCN4 peptide sequence facilitating helicity. In this regard, the skilled person is referred to Chapter 15 of CURRENT PROTOCOLS IN PROTEIN SCIENCE, Eds. Coligan et al. (John Wiley & Sons NY 1995-2008) for more extensive methodology relating to chemical modification of proteins.

As mentioned, an aspect of the invention provides a method of producing an immunogenic agent, including the step of covalently coupling a hydrophobic peptide to at least one antigenic molecule to thereby produce the immunogenic agent.

Suitably, the immunogenic agents may be produced by solid phase peptide synthesis (SPPS), a combination of SPPS and azide alkyne cycloaddition or any other chemical synthetic method that can couple the antigenic molecules (such as one or more epitopes) and the plurality of hydrophobic amino acids. Suitably, in some embodiments the hydrophobic peptide is coupled to each epitope through a carrier or linker, such as one comprising one or more lysine residues. As described in more detail hereinafter with reference to FIG. 1, peptide immunogenic agents may be synthesized by a microwave-assisted Boc-solid-phase peptide synthesis (SPPS) method. In some embodiments, the immunogenic agent is synthesized using classic solid phase peptide synthesis (SPPS).

As mentioned, an aspect of the invention provides a method of producing an immunogenic complex including the step of providing a plurality of immunogenic agents of the aforementioned aspects, whereby the plurality of immunogenic agents self-assemble into the immunogenic complex.

In particular embodiments, hydrophilic antigenic molecules upon covalent coupling or conjugation with hydrophobic peptide sequences form moieties/conjugates which have amphiphilic properties and therefore are able to self-assemble to form particles, such as nanoparticles or microparticles. Self-assembly may occur in any suitable environment (e.g., at suitable pH, salt concentration, temperature such as phosphate-buffered saline, although without limitation thereto). Self-assembly may be examined by dynamic light scattering (DLS), transmission electron microscopy (TEM) or any other methodology that facilitates detection of particles in a nanometer or micrometer size range (ie., nanoparticles or microparticles).

In some embodiments, self-assembly may involve forming chain-like aggregates of nanoparticles (CLAN) or microparticles.

In some embodiments, the hydrophobic peptide can induce the formation of alpha-helices and/or beta-sheets.

In some embodiments, the carrier or linker can induce the formation of alpha-helices and/or beta-sheets.

As mentioned, an aspect of the invention provides a method of eliciting an immune response to one or a plurality of pathogens in a subject. Preferably, the immune response is a protective immune response, whereby subsequent infection by the pathogen of interest is at least partly prevented or minimized.

As mentioned, an aspect of the invention provides a method of immunizing a subject against one or a plurality of pathogens.

As generally used herein “immunogenic” means capable of eliciting or inducing an immune response, such as upon administration to a subject. The elicited or induced immune response may include molecular and/or cellular elements of the innate and adaptive immune systems inclusive of NK cells, antigen-presenting cells such as dendritic cells, myelo-monocytic cells (e.g macrophages, eosinophils, neutrophils, granulocytes etc), lymphocytes inclusive of T cells and B cells, immunoreceptors such as antibodies and other cell surface molecules (e.g adhesion molecules, cytokine receptors, co-stimulatory molecules such as CD28, CD40, CD40L etc), cytokines, chemokines, growth factors and/or complement, although without limitation thereto.

As generally used herein “antigenic” means capable of binding, complexing with, or being detected or responded to by one or elements of the immune system. In some embodiments, an antigenic molecule may also be “immunogenic”, as described above.

For the purposes of this invention, by “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material may be in native, chemical synthetic or recombinant form.

By “protein” is meant an amino acid polymer. The amino acids may be natural or non-natural amino acids, D- or L-amino acids as are well understood in the art.

The term “protein” includes and encompasses “peptide”. A “peptide” typically has no more than fifty (50) amino acids, but this need not necessarily be the case. A “protein” typically has more than fifty (50) amino acids, but this need not necessarily be the case. The term “polypeptide” is typically used to describe a protein having more than one hundred (100) amino acids, but this need not be the case.

As generally used herein the terms “immunize”, “vaccinate” and “vaccine” refer to methods and/or compositions that elicit a protective immune response upon administration to a subject. The immune response may be protective, such as against a subsequent infection by a pathogen, or may have at least partial therapeutic efficacy once an infection has occurred.

As mentioned above, another aspect of the invention provides a method of treating or preventing a disease, disorder or condition in a subject.

As used herein, “treating”, “treat” or “treatment” refers to a therapeutic intervention that at least partly ameliorates, eliminates or reduces a symptom or pathological sign of disease, disorder or condition after it has begun to develop. Treatment need not be absolute to be beneficial to the subject. The beneficial effect can be determined using any methods or standards known to the ordinarily skilled artisan.

As used herein, “preventing”, “prevent” or “prevention” refers to a course of action initiated prior to disease onset (such as infection by, or exposure to, a pathogen or molecular components thereof) and/or before the onset of a symptom or pathological sign of the disease, disorder or condition, so as to prevent infection and/or reduce the symptom or pathological sign. It is to be understood that such preventing need not be absolute to be beneficial to a subject. A “prophylactic” treatment is a treatment administered to a subject who does not exhibit signs of the disease, disorder or condition, or exhibits only early signs for the purpose of decreasing the risk of developing a symptom or pathological sign of the disease, disorder or condition.

In one embodiment, the disease, disorder or condition is associated with, or caused by, an infection by the one or plurality of pathogens in the subject. Pathogens may be such as hereinbefore described.

In another embodiment, the disease, disorder or condition is a cancer. It will be appreciated that some cancers may be responsive to cancer immunotherapy by administration of the immunogenic agents comprising one or a plurality of same or different tumour antigens or fragments thereof. Non-limiting examples include MUC1 for metastatic cancers such as breast cancer, EBV for nasopharyngeal carcinoma, HPV for cervical cancer, hepatitis B and/or C for liver cancer, WT-1 for lung cancer, MAGE-1 for melanoma and PSA for prostate cancer, although without limitation thereto. A non-limiting review of tumour antigen-specific cancer treatments may be found in Tagliamonte et al., 2014, Hum. Vaccin. Immunother 10 3332.

As mentioned above, another aspect of the invention provides a composition comprising the immunogenic agent and/or the immunogenic complex hereinbefore described.

In a preferred form, the composition disclosed herein comprises an acceptable carrier, diluent or excipient.

By “acceptable carrier, diluent or excipient” is meant a solid or liquid filler, diluent or encapsulating substance that may be safely used in systemic administration. Depending upon the particular route of administration, a variety of carriers, diluent and excipients well known in the art may be used. These may be selected from a group including sugars, starches, cellulose and its derivatives, malt, gelatine, talc, glidants, calcium sulfate, vegetable oils, synthetic oils, polyols, alginic acid, phosphate buffered solutions, emollients, emulsifiers, glidants, isotonic saline and salts such as mineral acid salts including hydrochlorides, bromides and sulfates, organic acids such as acetates, propionates and malonates, water and pyrogen-free water.

A useful reference describing acceptable carriers, diluents and excipients is Remington's Pharmaceutical Sciences (Mack Publishing Co. N.J. USA, 1991) which is incorporated herein by reference.

The composition may, or may not comprise an adjuvant. In some embodiments, an adjuvant may not be necessary due to the “self-adjuvanting” properties of the immunogenic agent. In some embodiments, the methods do not further include administering an adjuvant to the subject. In some embodiments, the methods/composition further includes administering an adjuvant to the subject. Non-limiting examples of adjuvants include Freund's adjuvant, aluminium hydroxide (alum), aluminium phosphate, squalene, IL-12, CpG-oligonucleotide, Montanide ISA720, imiquimod, SBAS2, SBAS4, MF59, MPL, Quil A, QS21 and ISCOMs.

Any suitable procedure is contemplated for producing compositions such as vaccine formulations. Exemplary procedures include, for example, those described in New Generation Vaccines (1997, Levine et al., Marcel Dekker, Inc. New York, Basel, Hong Kong), which is incorporated herein by reference.

Any safe route of administration may be employed, including intranasal, oral, rectal, parenteral, sublingual, buccal, intravenous, intra-articular, intra-muscular, intra-dermal, subcutaneous, inhalational, intraocular, intraperitoneal, intracerebroventricular, topical, mucosal and transdermal administration, although without limitation thereto.

In particular embodiments, the composition is suitable for delivery of the immunogenic agent and/or the immunogenic complex to a mucosal epithelium, such as by oral administration, for delivering a desired antigenic molecule across the mucosal epithelium. The composition of the present aspect may thus be used to deliver one or more antigen molecules across intestinal epithelial tissue, lung epithelial tissue and other mucosal surfaces including nasal surfaces, vaginal surfaces, ocular surfaces and colon surfaces so as to induce mucosal immune responses and/or systemic immune responses.

Dosage forms include tablets, dispersions, suspensions, injections, solutions, syrups, troches, capsules, nasal sprays, suppositories, aerosols, transdermal patches and the like. These dosage forms may also include injecting or implanting controlled release devices designed specifically for this purpose or other forms of implants modified to act additionally in this fashion. Controlled release may be effected by coating with hydrophobic polymers including acrylic resins, waxes, higher aliphatic alcohols, polylactic and polyglycolic acids and certain cellulose derivatives such as hydroxypropylmethyl cellulose.

Compositions may be presented as discrete units such as capsules, sachets, functional foods/feeds or tablets each containing a pre-determined amount of one or more therapeutic agents of the invention, as a powder or granules or as a solution or a suspension in an aqueous liquid, a non-aqueous liquid, lipid particles or vesicles such as liposomes, micelles or minicells, an oil-in-water emulsion or a water-in-oil liquid emulsion. Such formulations may be prepared by any pharmaceutical methods but all methods include the step of bringing into association one or more agents as described above with the carrier which constitutes one or more necessary ingredients. In general, the formulations are prepared by uniformly and intimately admixing the agents of the invention with liquid carriers or finely divided solid carriers or both, and then, if necessary, shaping the product into the desired presentation.

In some embodiments, the composition is formulated as a liposomal formulation or vaccine. A “liposome” is a small vesicle composed of various types of lipids, phospholipids and/or surfactant which is useful for delivery of a drug (such as the immunogenic agent and/or the immunogenic complex disclosed herein) to a subject. The components of the liposome are commonly arranged in a bilayer formation, similar to the lipid arrangement of biological membranes. The liposomal formulation may be prepared by any means known in the art.

The present invention further contemplates active targeting, which involves the use of additional excipients, referred to herein as “targeting ligands” that may be bound (either covalently or non-covalently) to the liposome or other transfer vehicle to encourage localization of such liposome or transfer vehicle at certain target cells or target tissues (e.g., immune cells). For example, targeting may be mediated by the inclusion of one or more endogenous targeting ligands (e.g., mannose or a fragment or derivative thereof) in or on the liposome or transfer vehicle to encourage distribution to the target cells or tissues. Recognition of the targeting ligand by the target tissues actively facilitates tissue distribution and cellular uptake of the transfer vehicle and/or its contents in the target cells and tissues (e.g., the inclusion of a mannose targeting ligand in or on the liposome or transfer vehicle encourages recognition and binding of the transfer vehicle to mannose receptors expressed by immune cells, such as antigen presenting cells, as hereinafter described). To this end, mannosylated liposomes can be considered as promising non-live vectors for the targeted delivery of therapeutic agents, such as the composition, the immunogenic agent and/or the immunogenic complex disclosed herein.

The above formulations may be administered in a manner compatible with the dosage formulation, and in such an amount that is effective. The dose administered to a patient, in the context of the present invention, should be sufficient to effect a beneficial response in a patient over an appropriate period of time. The quantity of agent(s) to be administered may depend on the subject to be treated inclusive of the age, sex, weight and general health condition thereof, factors that will depend on the judgement of the practitioner.

The subject may be any suitable type of animal. For example, the subject may be a fish, bird, amphibian, reptile or mammal. The subject may be a vertebrate. In some embodiments, the subject is a mammal. As generally used herein, the terms “patient”, “individual” and “subject” are used in the context of any animal recipient of a treatment or formulation disclosed herein. Accordingly, the methods and formulations disclosed herein may have medical and/or veterinary applications, such as in humans, performance animals (such as horses, camels and greyhounds), livestock (such as cows, sheep, horses, pigs and chickens), birds and poultry (such as chickens), companion animals (such as cats and dogs), and laboratory animals (such as mice, rats, rabbits and primates). In a preferred form, the mammal is a human.

Preferred embodiments of the invention are described in the paragraphs below.

1. An immunogenic agent suitable for administration to a subject, said immunogenic agent comprising a hydrophobic peptide covalently coupled or conjugated to at least one antigenic molecule.

2. An immunogenic agent having the structure: [Antigenic molecule]ₙ-[Carrier/Linker]ₘ-[Hydrophobic peptide],

wherein: n is 1 or a higher number;

m is 0 or a higher number; and

“-” represents a covalent coupling or conjugation.

3. A method of producing an immunogenic agent, including the step of covalently coupling a hydrophobic peptide to at least one antigenic molecule to thereby produce the immunogenic agent.

4. An immunogenic agent when produced by the method according to paragraph 3.

5. An immunogenic complex comprising a plurality of immunogenic agents according to any one of paragraphs 1, 2 and 4, wherein a plurality of hydrophobic peptides interact in the immunogenic complex.

6. A method of producing an immunogenic complex comprising the step of combining a plurality of immunogenic agents of any one of paragraphs 1, 2 and 4, whereby the plurality of immunogenic agents self-assemble into the immunogenic complex.

7. An immunogenic complex when produced by the method of paragraph 6.

8. A composition comprising at least one said immunogenic agent of any one of paragraphs 1, 2 and 4, and/or at least one said immunogenic complex of paragraph 5 or paragraph 7.

9. A method of eliciting an immune response in a subject, said method comprising the step of administering to the subject at least one said immunogenic agent of any one of paragraphs 1, 2 and 4, at least one said immunogenic complex of paragraph 5 or paragraph 7, or at least one said composition of paragraph 8, to thereby elicit an immune response in the subject.

10. A method of immunizing a subject, said method comprising the step of administering to the subject at least one said immunogenic agent of any one of paragraphs 1, 2 and 4, at least one said immunogenic complex of paragraph 5 or paragraph 7, or at least one said composition of paragraph 8, to thereby immunize the subject.

11. A method of treating or preventing a disease, disorder or condition in a subject, said method comprising the step of administering to the subject at least one said immunogenic agent of any one of paragraphs 1, 2 and 4, at least one said immunogenic complex of paragraph 5 or paragraph 7, or at least one said composition of paragraph 8.

Context permitting, the invention as described above has the following features.

The invention as described above, wherein the hydrophobic peptide comprises a plurality of hydrophobic natural amino acids.

The invention as described above, wherein the hydrophobic peptide comprises, consists essentially of, or consists of anywhere between 2 and about 50 amino acids.

The invention as described above, wherein, the hydrophobic amino acids is selected from the group consisting of: glycine, proline, valine, alanine, phenylalanine and leucine.

The invention as described above, wherein the hydrophobic peptide consists of 10 valine residues, 10 phenylalanine residues, 10 leucine residues, 15 leucine residues, 15 glycine residues, 15 proline residues, 15 alanine residues, 25 alanine residues, or 5 phenylalanine-leucine-alanine tri-peptide residues.

The invention as described above, wherein when the hydrophobic peptide consists of or comprises polyleucine, the hydrophobic peptide allows anchoring of the immunogenic agent to a liposomal membrane.

The invention as described above, wherein the conformation of the at least one antigenic molecule (epitope) is controlled by changing the amino acid sequence and/or length of the hydrophobic peptide.

The invention as described above, wherein the solubility of the immunogenic agent is controlled by changing the amino acid sequence and/or length of the hydrophobic peptide.

The invention as described above, wherein the immunogenic agent further comprises at least one carrier or linker to which the hydrophobic peptide and at least one antigenic molecule are directly or indirectly covalently coupled or conjugated.

The invention as described above, wherein the immunogenic agent is essentially linear (not branched).

The invention as described above, wherein the immunogenic agent is branched or dendritic in structure.

The invention as described above, wherein when the immunogenic agent comprises a plurality of antigenic molecules, the carrier or linker is utilised to couple or connect the antigenic molecules together in a contiguous or branched form or manner.

The invention as described above, wherein the at least one carrier or linker balances the hydrophobic:hydrophilic ratio of the immunogenic agent to assist with proper conformation of the immunogenic agent/at least one antigenic molecule, and/or solubility of the immunogenic agent.

The invention as described above, wherein the carrier or linker induces the formation of alpha-helices and/or beta-sheets.

The invention as described above, wherein the at least one carrier or linker provides the immunogenic agent with a linear structure.

The invention as described above, wherein the at least one carrier or linker provides the immunogenic agent with a branched or dendritic structure.

The invention as described above, wherein the at least one carrier or linker comprises at least one amino acid that is capable of forming a branched chain, for attachment to the hydrophobic peptide.

The invention as described above, wherein the at least one amino acid that is capable of forming a branched chain is lysine or cysteine.

The invention as described above, wherein the at least one carrier or linker comprises the peptide lysine-lysine-lysine-lysine-serine-serine (SEQ ID NO:5).

The invention as described above, wherein the at least one carrier or linker comprises the peptide serine-lysine-lysine-lysine-lysine (SEQ ID NO:6).

The invention as described above, wherein any or all N-terminal ends of the immunogenic agent are acetylated.

The invention as described above, wherein any or all C-terminal ends of the immunogenic agent are amidated.

The invention as described above, wherein the antigenic molecule is a protein, peptide, carbohydrate, lipid or combination of these such as a glycoprotein, proteoglycan, lipoprotein, glycolipoprotein or fragment or derivative thereof.

The invention as described above, comprising more than one antigenic molecule.

The invention as described above, wherein the at least one antigenic molecule comprises, consists essentially of, or consists of a peptide or a protein.

The invention as described above, wherein the antigenic molecule is, or comprises, a synthetic epitope that is not derived from, or directed to, a particular pathogen.

The invention as described above, wherein the at least one antigenic molecule consists of or comprises a B-cell epitope.

The invention as described above, wherein the at least one antigenic molecule consists of or comprises a T-cell epitope.

The invention as described above, wherein the at least one antigenic molecule comprises both a B cell epitope and a T cell epitope.

The invention as described above, wherein the at least one antigenic molecule consists of or comprises a non-natural Pan-DR-helper cell epitope (PADRE; amino acid sequence: AKFVAAWTLKAAA; SEQ ID NO:7).

The invention as described above, wherein the at least one antigenic molecule consists of or comprises P25 (amino acid sequence: KLIPNASLIENCTKAEL; SEQ ID NO:8).

The invention as described above, wherein the at least one antigenic molecule, or fragment thereof, is of a pathogen.

The invention as described above, wherein the pathogen is selected from a virus, bacteria, fungus, yeast, protist or worms.

The invention as described above, wherein the at least one antigenic molecule consists of or comprises a peptide derived from Group A Streptococcus (GAS) major virulent factor M-protein.

The invention as described above, wherein the at least one antigenic molecule consists of or comprises a peptide derived from the C repeat region of the M-protein.

The invention as described above, wherein the at least one antigenic molecule consists of or comprises a J8 peptide (QAEDKVKQSREAKKQVEKALKQLEDKVQ; SEQ ID NO:9) derived from Group A Streptococcus (GAS) major virulent factor M-protein.

The invention as described above, wherein the at least one antigenic molecule consists of or comprises the PL1 B-cell epitope (EVLTRRQSQDPKYVTQRIS; SEQ ID NO:10) derived from the N-terminal of M protein of GAS strain M54.

The invention as described above, wherein the at least one antigenic molecule consists of or comprises the 88/30 B-cell epitope (DNGKAIYERARERALQELGP; SEQ ID NO:11) derived from the N-terminal of M protein of GAS strain 88/30.

The invention as described above, wherein the at least one antigenic molecule consists of or comprises a synthetic epitope that is not derived from or directed to a particular pathogen, and an epitope from a particular pathogen.

The invention as described above, wherein the immunogenic agent comprises a hydrophobic 10 to 15 amino acid peptide covalently coupled or conjugated to the GAS B-cell epitope J8 and the universal T-helper epitope PADRE.

The invention as described above, wherein the immunogenic agent comprises a hydrophobic 10 amino acid peptide, preferably consisting of polyleucine, covalently coupled or conjugated to the GAS B-cell epitope J8 and the universal T-helper epitope PADRE.

The invention as described above, wherein the immunogenic agent comprises a hydrophobic 15 amino acid peptide, preferably consisting of polyleucine, covalently coupled or conjugated to the universal T-helper epitope PADRE which, in turn, is coupled or conjugated to the GAS B-cell epitope J8.

The invention as described above, wherein the immunogenic agent has the structure of Compound 5 of FIG. 1:

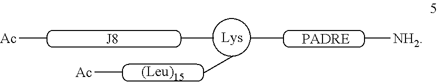

The invention as described above, wherein the immunogenic agent has the structure of Compound 7 of FIG. 22 b) (SEQ ID NO:26).

The invention as described above, wherein the immunogenic agent comprises a hydrophobic 10 to 15 amino acid peptide covalently coupled or conjugated to the GAS B-cell epitope PL1 and the universal T-helper epitope PADRE.

The invention as described above, wherein the immunogenic agent has the structure of Compound 8 of FIG. 22 b) (SEQ ID NO:27).

The invention as described above, wherein the immunogenic agent comprises a hydrophobic 10 to 15 amino acid peptide covalently coupled or conjugated to the GAS B-cell epitope 88/30 and the universal T-helper epitope PADRE.

The invention as described above, wherein the immunogenic agent has the structure of Compound 9 of FIG. 22 b) (SEQ ID NO:28).

The invention as described above, wherein the immunogenic agent comprises a hydrophobic 10 to 15 amino acid peptide covalently coupled or conjugated to the GAS B-cell epitopes J8, PL1 and 88/30, and the universal T-helper epitope PADRE.

The invention as described above, wherein the immunogenic agent has the structure of Compound 14 of FIG. 22 b):

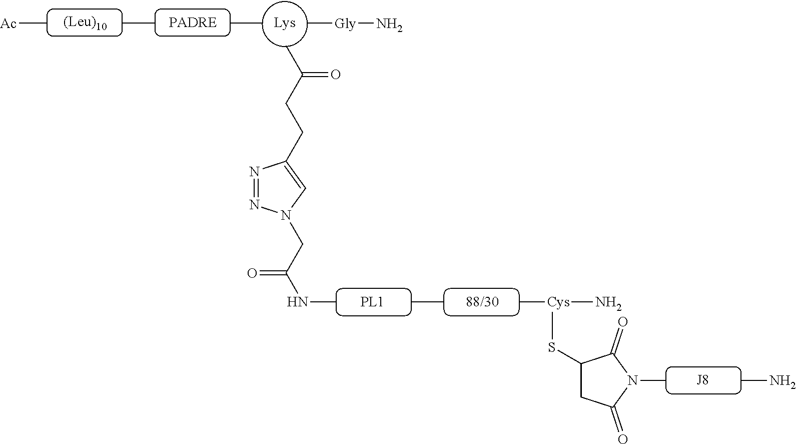

The invention as described above, wherein Compounds 7, 8 and 9 of FIG. 22 b) are combined to form the immunogenic complex:

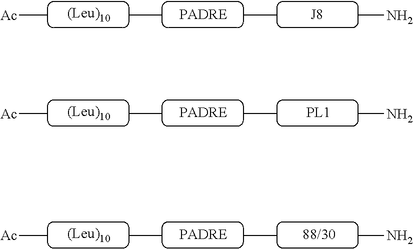

The invention as described above, wherein the immunogenic agent has the structure of one of the following:

Ac-J8-K(Ac-(Leu)₁₅)PADRE, wherein “Ac” denotes acetylation and “K” denotes lysine;

Ac-(Leu)₁₅-PADRE-K-J8, wherein “Ac” denotes acetylation and “K” denotes lysine (SEQ ID NO:29);

Ac-(Leu)₁₅-J8-K-PADRE, wherein “Ac” denotes acetylation and “K” denotes lysine (SEQ ID NO:30);

Ac-(Leu)₁₅-PADRE-J8, wherein “Ac” denotes acetylation (SEQ ID NO:31);

Ac-(Glu)₁₅-PADRE-J8, wherein “Ac” denotes acetylation (SEQ ID NO:32);

Ac-(Gly)₁₅-PADRE-J8, wherein “Ac” denotes acetylation (SEQ ID NO:33);

Ac-(Ser)₁₅-PADRE-J8, wherein “Ac” denotes acetylation (SEQ ID NO:34);

Ac-(Pro)₁₅-PADRE-J8, wherein “Ac” denotes acetylation (SEQ ID NO: 35);

Ac-(Ala)₁₅-PADRE-J8, wherein “Ac” denotes acetylation (SEQ ID NO:36); or

Ac-(Phe-Leu-Ala)₅-PADRE-J8, wherein “Ac” denotes acetylation (SEQ ID NO:37).

The invention as described above, wherein the immunogenic agent has the structure of Compound 2:

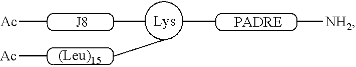

or the structure of any one of Compounds 3 to 11 of FIG. 26 (SEQ ID NOs:29-37).

The invention as described above, wherein the at least one antigenic molecule consists of or comprises a peptide derived from a spike protein of a coronavirus.

The invention as described above, wherein the at least one antigenic molecule consists of or comprises a peptide derived from SARS-CoV-2 spike protein.

The invention as described above, wherein the at least one antigenic molecule consists of or comprises: ACE2-RBD critical binding amino acid residues; amino acid residues at or near S1/S2 cleavage/priming site; amino acid residues at receptor binding motif of RBD; or amino acid residues at NTD of receptor binding domain but not in direct contact with ACE2.

The invention as described above, wherein the at least one antigenic molecule consists of or comprises the SARS-CoV spike protein epitope AIHADQLTPTWRVYSTG (S₆₂₃₋₆₃₉) (SEQ ID NO:15).

The invention as described above, wherein the at least one antigenic molecule consists of or comprises the SARS-CoV spike protein epitope STEIYQAGSTPCNGVEGFNCYFPLQSYGFQPTNGVGYQPY (S₄₆₉₋₅₀₈) (SEQ ID NO:16).

The invention as described above, wherein the at least one antigenic molecule consists of or comprises the SARS-CoV spike protein epitope VGGNYNYLYRLFRKSNLKPFERDISTEIYQAGSTPCNGV (S₄₄₅₋₄₈₃) (SEQ ID NO:17).

The invention as described above, wherein the at least one antigenic molecule consists of or comprises the SARS-CoV spike protein epitope FLPFQQFGRDIADT (S₅₅₉₋₅₇₂) (SEQ ID NO:18).

The invention as described above, wherein the at least one antigenic molecule consists of or comprises the SARS-CoV spike protein epitope SVLYNSASFSTFKCYGVSPTKLNDLCFTNV (S₃₆₆₋₃₉₅) (SEQ ID NO:19).

The invention as described above, wherein the immunogenic agent comprises a hydrophobic 10 to 15 amino acid peptide covalently coupled or conjugated to an SARS-CoV spike protein epitope.

The invention as described above, wherein the immunogenic agent comprises a hydrophobic 10 to 15 amino acid peptide covalently coupled or conjugated to an SARS-CoV spike protein epitope and the T-helper epitope PADRE (SEQ ID NO:7).

In some embodiments, the immunogenic agent comprises a hydrophobic 10 to 15 amino acid peptide covalently coupled or conjugated to an SARS-CoV spike protein epitope and at least one carrier or linker, preferably a peptide or protein.

The invention as described above, wherein the immunogenic agent comprises a hydrophobic 10 to 15 amino acid peptide covalently coupled or conjugated to VGGNYNYLYRLFRKSNLKPFERDISTEIYQAGSTPCNGV (SEQ ID NO:17).

The invention as described above, wherein the hydrophobic peptide consists of leucine, valine, phenylalanine, glycine, proline, alanine, phenylalanine-leucine-alanine tri-peptide repeat residues, or any combination of these.

The invention as described above, wherein the immunogenic agent is LLLLLLLLLLVGGNYNYLYRLFRKSNLKPFERDISTEIYQAGSTPCNGV (Compound (Leu)₁₀-B3; SEQ ID NO:38).

The invention as described above, wherein the immunogenic agent comprises a hydrophobic 10 to 15 amino acid peptide covalently coupled or conjugated to the universal T-helper epitope PADRE (SEQ ID NO:7).

The invention as described above, wherein the immunogenic agent comprises a hydrophobic 10 leucine amino acid peptide covalently coupled or conjugated to the universal T-helper epitope PADRE (Compound (Leu)₁₀-PADRE) (SEQ ID NO:39).

The invention as described above, wherein Compounds (Leu)₁₀-B3 (SEQ ID NO:38) and (Leu)₁₀-PADRE (SEQ ID NO:39) are combined to form the immunogenic complex.

The invention as described above, wherein the immunogenic agent or immunogenic complex are delivered in a composition comprising liposomes.

The invention as described above, wherein the immunogenic agent or immunogenic complex are anchored into the liposomes by way of the hydrophobic peptide.

The invention as described above, wherein the immunogenic agent or immunogenic complex are delivered in a composition comprising liposomes and a mannose targeting moiety incorporated into the liposomes.

The invention as described above, wherein the at least one antigenic molecule is, or comprises, a tumour antigen or fragment thereof.

The invention as described above, wherein the tumour antigen is a protein, glycoprotein, a carbohydrate-containing fragment, lipoprotein or other molecule expressed by tumour cells.

The invention as described above, wherein the at least one antigenic molecule consists of or comprises a peptide derived from an oncoprotein.

The invention as described above, wherein the at least one antigenic molecule consists of or comprises a peptide derived from a HPV oncoprotein.

The invention as described above, wherein the at least one antigenic molecule consists of or comprises a peptide derived from the HPV oncoprotein called E7.

The invention as described above, wherein the at least one antigenic molecule consists of or comprises a CD8+ peptide.

The invention as described above, wherein the at least one antigenic molecule consists of or comprises 8Qm (E7_{44_57}, QAEPDRAHYNIVTF; SEQ ID NO:14), derived from HPV-16 E7 oncoprotein.

The invention as described above, wherein the immunogenic agent comprises a hydrophobic 10 to 15 amino acid leucine polypeptide covalently coupled or conjugated to 8Qm (SEQ ID NO:14).

The invention as described above, wherein the immunogenic agent has the structure of Compound Leu-8Qm of FIG. 14 b) (LLLLLLLLLLQAEPDRAHYNIVTF; SEQ ID NO:25).

The invention as described above, wherein the immunogenic agent or immunogenic complex is delivered in a composition comprising liposomes.

The invention as described above, wherein the immunogenic agent or immunogenic complex is anchored into the liposomes by way of the hydrophobic peptide.

The invention as described above, wherein the immunogenic agent or immunogenic complex is delivered in a composition comprising liposomes and a mannose targeting moiety incorporated into the liposomes.

The invention as described above, wherein the at least one antigenic molecule consists of or comprises a peptide derived from a hookworm.

The invention as described above, wherein the at least one antigenic molecule consists of or comprises a Necator americanus peptide derived from Na-APR-1, called A₂₉₁Y.

The invention as described above, wherein the at least one antigenic molecule consists of or comprises a B cell epitope called p3 (TSLIAGPKAQVEAIQKYIGAEL; SEQ ID NO:12).

The invention as described above, wherein the immunogenic agent comprises a hydrophobic 10 to 15 amino acid peptide covalently coupled or conjugated to the epitope p3 (SEQ ID NO:12) and the universal T-helper epitope P25 (SEQ ID NO:8).

The invention as described above, wherein the immunogenic agent comprises a hydrophobic 10 amino acid peptide, preferably consisting of polyleucine, covalently coupled or conjugated to the epitope p3 (SEQ ID NO:12) and the universal T-helper epitope P25 (SEQ ID NO:8).

The invention as described above, wherein the immunogenic agent further comprises at least one carrier or linker, preferably a lysine branching spacer attached to the hydrophobic peptide, and a hydrophilic moiety built from lysine and serine to increase the solubility and allow for self-assembly of the epitopes.

The invention as described above, wherein the immunogenic agent has the structure of Compound 3 of FIG. 18:

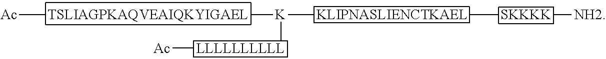

The invention as described above, wherein the immunogenic agent or immunogenic complex is suitable for oral administration to the subject.

The invention as described above, wherein the immunogenic agent is synthesized using classic solid phase peptide synthesis (SPPS).

The invention as described above, wherein said hydrophilic antigenic molecules upon covalent coupling or conjugation with hydrophobic peptide sequences form moieties/conjugates which have amphiphilic properties and therefore are able to self-assemble to form particles, such as nanoparticles or microparticles.

The invention as described above, wherein self-assembly involves forming chain-like aggregates of nanoparticles (CLAN) or microparticles.

The invention as described above, wherein the hydrophobic peptide can induce the formation of alpha-helices and/or beta-sheets.

The invention as described above, wherein the carrier or linker induces the formation of alpha-helices and/or beta-sheets.

The invention as described above, wherein the hydrophobic peptide enables anchoring of the immunogenic agent to a liposomal membrane, or other lipophilic surface such as a micelle or nanoparticle.

The invention as described above, wherein the hydrophobic amino acids are preferably selected from the group consisting of: polyglycine, polyproline, polyvaline, polyalanine, polyphenylalanine and polyleucine.

The invention as described above, wherein the at least one antigenic molecule comprises, consists essentially of, or consists of a peptide, a protein or carbohydrate, or a fragment or a derivative thereof.

The invention as described above, the at least one antigenic molecule and the hydrophobic peptide are situated at opposed termini of the immunogenic agent.

The invention as described above, wherein when the immunogenic agent comprises a plurality of antigenic molecules, a carrier or linker is utilised to couple or connect the antigenic molecules together in a contiguous, branched or dendritic form or manner.

The invention as described above, wherein the composition further comprises:

liposomes, micelles or nanoparticles; or

liposomes, micelles or nanoparticles, and a mannose targeting moiety.

The invention as described above, wherein the animal is a mammal, bird, pig or human; and/or the method does not require co-administration of an adjuvant or other general immune-stimulant.

By “consisting essentially of” in the context of an amino acid sequence is meant the recited amino acid sequence together with an additional one, two or three amino acids at the N- or C-terminus.

Unless the context requires otherwise, the terms “comprise”, “comprises” and “comprising”, or similar terms are intended to mean a non-exclusive inclusion, such that a recited list of elements or features does not include those stated or listed elements solely, but may include other elements or features that are not listed or stated.

As used herein, the indefinite articles ‘a’ and ‘an’ are used here to refer to or encompass singular or plural elements or features and should not be taken as meaning or defining “one” or a “single” element or feature.

### BRIEF DESCRIPTION OF THE DRAWINGS

FIG. 1. Schematic structures of Compounds 1-5. The vaccine candidates were constructed using three general building blocks: the B-cell epitope (J8; SEQ ID NO:9); the T-helper epitope (PADRE; SEQ ID NO:7); and a poly-hydrophobic amino acids (pHAAs) unit (ie. (Val)₁₀, (Phe)₁₀, (Leu)₁₀ or (Leu)₁₅). Also shown is a branched carrier comprising a single lysine (Lys) residue. The N-termini are acetylated (“Ac”).

FIG. 2. Physicochemical characterization of Compounds 2-5. Transmission electron microscopy photographs of the vaccine Compounds (a) 2, (b) 3, (c) 4, and (d) 5 (bar 200 nm). (e) Circular dichroism spectra of Compounds 1-5.

FIG. 3. Expression of APC maturation markers MHC-II and CD40 on CD11c⁺ dendritic cells (“DCs”) and F4/80⁺ macrophages in response to stimulation with vaccine candidates 2 to 5. Bar represents the intensity of mean fluorescence of CD11c/F4/80 and CD40/CD86/MHC-II double positive cells. (a) Mean fluorescence intensity (MFI (±SD)) of isolated CD11c⁺ dendritic cells and F4/80+ macrophages for CD40 expression; (b) Mean fluorescence intensity (MFI (±SD)) of isolated CD11c⁺ dendritic cells and F4/80⁺ macrophages for MHC-II expression. Statistical analysis was performed using two-way ANOVA followed by Tukey's multiple comparison test compared with PBS as indicated (ns, p>0.05; *p<0.05; **p<0.01; ***p<0.001; ****p<0.0001).

FIG. 4. Experimental mice (C57BL/6, n=10) were vaccinated subcutaneously (on days 0, 21, 28 and 35) with Compounds 1-5. Each triangle in (a)-(c) represents an individual mouse; the mean J8-specific titers are represented as a bar. (a) Titers of J8-specific IgG in serum; (b) titers of J8-specific IgA in saliva; and (c) titers of J8-specific IgG in saliva, collected after final immunization. (d) Average percentage opsonisation of GAS strain 2002; (e) a D2612; (f) D3840; (g) GC2203; (h) 5230; (i) 5199. PBS: negative control group, mice immunised with phosphate-buffered saline; 1/CFA: positive control group, Compound 1 emulsified with CFA. Statistical analysis was performed using one-way ANOVA followed by Tukey's post hoc test compared with PBS as indicated (ns, p>0.05; *, p<0.05; **, p<0.01; ***, p<0.001).

FIG. 5. Bacterial burden after intranasal challenge with M1 GAS strain in C57BL/6 mice (n=10/group). Bacterial burden results are represented as the mean CFU±SEM for the 10 mice/group. (a) Nasal shedding; (b) Throat swabs; (c) Colonization of NALT; and (d) spleen. PBS: negative control group, mice immunised with phosphate-buffered saline; 1/CFA: positive control group, Compound 1 emulsified with CFA. Statistical analysis was performed using two-way ANOVA followed by Tukey's post hoc test compared with PBS as indicated (ns, p>0.05; *, p<0.05; **, p<0.01; ***, p<0.001).

FIG. 6. (a) MS and (b) HPLC spectra of Compound 1.

FIG. 7. (a) MS and (b) HPLC spectra of Compound 2.

FIG. 8. (a) MS and (b) HPLC spectra of Compound 3.

FIG. 9. (a) MS and (b) HPLC spectra of Compound 4.

FIG. 10. (a) MS and (b) HPLC spectra of Compound 5.

FIG. 11. Photographs of mouse tails 55 days post-primary immunisation. A. Tails of mice from the positive group that had CFA-adjuvanted peptide epitopes injected; B. Tails of mice from the group that had vaccine candidate 19 injected. The scars on the injection site (inflammation) were visible even after 55 days post-CFA immunisation (FIGS. 4 and 5).

FIG. 12. Latex agglutination test results of bacteria collected from challenge experiment. A, group A Streptococcus; B, group B Streptococcus; C, group C Streptococcus; D, group D Streptococcus; F, group F Streptococcus; G, group G Streptococcus; +, group A Streptococcus. Presence of dotted spots (and not solid) confirmed presence of group A Streptococcus.

FIG. 13. Generalized structure of an immunogenic agent.

FIG. 14. Schematic representation of vaccine candidates used in the study: (a) D-8Q; (b) Leu-8Q; (c) DOPE-PEG3.4K-mannose; (d) Li; (e) L2; and (f) L2M.

FIG. 15. Scheme depicting synthesis of DOPE-PEG3.4K-mannose (10).

FIG. 16. Transmission electron microscopy images of (a) L1, (b) L2, and (c) L2M stained with 2% uranyl acetate (bar=200 nm).

FIG. 17. In vivo tumour treatment experiments and CD8+ T-cell activation. C57BL/6 mice (n=8 mice/group) were inoculated subcutaneously with TC-1 tumour cells (day 0) and vaccinated with different immunogens on day 7. a) Survival rate was monitored and time to death plotted on a Kaplan-Meier survival curve. Tumour volume was monitored and plotted for each individual mouse immunised with L2 (b) and L2M (c). d) Assessment of CD8+ T-cell response to vaccination. Mice were subcutaneously immunised with the vaccine candidates in both flanks. Ten days later, spleens were harvested and IFN-7 production in response to short E7₄₉₋₅₇ (RAHYNIVTF; SEQ ID NO:13) peptide was determined by ELISPOT (n=6 mice/group). The L3 group was J8 derived from GAS attached to polyleucine, encapsulated into liposomes (irrelevant control). The data were pooled from two independent experiments and analyzed using one-way ANOVA followed by Tukey's post hoc multiple comparison test (*, p<0.05; **, p<0.01; ***, p<0.001; ****, p<0.0001).

FIG. 18. Schematic representation of vaccine peptides 1 (a), 2 (b) and 3 (c). Vaccine candidates consisted of Na p3 peptide (SEQ ID NO:12) derived from hookworm, and a T helper P25 (SEQ ID NO:8) (a), conjugated to a LCP delivery system with KKKKSS peptide (SEQ ID NO:5) as hydrophilic linker (b) or conjugated to a polyleucine delivery system with a similar hydrophilic linker (c) (SKKKK; SEQ ID NO:6).

FIG. 19. Transmission electron microscopy images of peptide 2 (a) and peptide 3 (b) stained with 2% uranyl acetate.

FIG. 20. p3-specific, Na-APR-1 and Nbr ESP IgG antibody titers following oral immunisation from pre-challenge blood (day 48) and 7 days post challenge with N. brasiliensis (day 56) analysed using ELISA. (a) p3-specific serum IgG titers at day 48, (b) Na-APR-1-specific serum IgG titers at day 48, (c) Nbr ESP-specific serum IgG titers at day 48, (d) p3-specific serum IgG titers at day 56, (e) Na-APR-1-specific serum IgG titers at day 56, and (f) Nbr ESP-specific serum IgG titers at day 56. Blood was collected from tail bleed (pre-challenge) or cardiac puncture after challenge (at euthanasia). Each point represents an individual inbred BALB/c mouse (n=10). Statistical analysis was performed using one-way ANOVA followed by the Tukey Post Hoc test compared to PBS as indicated (ns, p>0.05; *, p<0.05; **, p<0.01; ***, p<0.001, ****, p<0.0001).

FIG. 21. Peptides 1, 2 and 3 induce highly significant reductions in parasite burden after challenge with N. brasiliensis. Number of worms in the small intestines (a) and number of eggs in faeces collected from the colon (b) were significantly reduced compared to control mice that received PBS. The egg burden was calculated based on the amount of eggs in a gram of faeces. Horizontal bars represent the mean of each group. Statistical analysis was performed using non-parametric Mann-Whitney U test (*, p<0.05; **, p<0.01; ***, p<0.001; ****, p<0.0001). Statistical analyses compared mice immunized with peptide antigens with PBS-treated mice.

FIG. 22. The structure of peptide epitopes (1-6) and pHAAs-antigen conjugate used to compare conjugation (14) vs. mix strategy (7-8).

FIG. 23. Particle-imaging and morphology of Compounds 4-6 (mixture), 7-9 (mixture) and individual Compound 7-9 and Compound 14, captured by TEM (bar 500 nm). Individual Compound 4-6 formed polydisperse nanoparticles, which mainly detected the peptide itself (DLS) no TEM. On the other hand, mixture of Compound 4-6 formed larger polydisperse nanoparticles (120 and 700 nm), with more defined combination of small nanoparticles and CLAN from TEM. Compound 7 self-assembled into small nanoparticles and CLAN (TEM image) with particle size (320 nm) from DLS. Similar particle size was measured by DLS for Compound 9. However, TEM image for Compound 9 shows small nanoparticle with slightly larger and more visible aggregates compared to Compound 7, which has similar morphology to Compound 8 that have bigger particle size (530 nm). Mixture of Compounds 7-9 formed large particles of size 600 nm than multiepitopes-conjugate (Compound 14) with 350 nm aggregates measured by DLS. TEM results are needed for the complete evaluation on the physiochemical characteristic and morphologies of mixed compounds and multiepitopes compound.

FIG. 24. J8-, PL1-, and 88/30-specific serum IgG antibody titers present in the final bleed after immunisation with Compounds 4-6 (mixture)+CFA (positive control), 2-3 (mixture) and 14 in C57BL/6 mice, as analysed by ELISA. Each point represents individual mice; a bar represents the average antigen-specific serum IgG antibody titers. Statistical analysis was performed using one-way ANOVA with Tukey's multiple comparison test ((*) P<0.05, (**) P<0.01, (***) P<0.001, (****) P<0.0001).

FIG. 25. Analysis of the purified Compounds 1-14 by analytical RP-HPLC (a) and ESI-MS (b). The compounds were purified using preparative RP-HPLC with solvent B concentration gradient 25-45% (1-3 and 10-11), 30-50% (4-6), 55-75% (7-9), and 70-90% (13) from Rₜ 5 min to 30 min. Analytical RP-HPLC graphs show pure compounds in single peak. The mass from these peaks, matched to the desired compounds in ESI-MS.

FIG. 26. The structure of (a) peptide epitopes; (b) the vaccine constructs with different epitope arrangements; and (c) different pHAA sequences.

FIG. 27. Particle images of compounds 2-8 and 10-11, captured by TEM (bar 200 nm; negative staining from 2% phosphotungstic acid visible as dark areas).

FIG. 28. J8-specific serum IgG antibody titers in C57BL/6 mice (n=5) following immunization with compounds 1+CFA and 2-4, as determined by ELISA. Each point represents an individual mouse; bars represent the average antigen-specific serum IgG antibody titers.

Statistical analysis was performed using one-way ANOVA with Tukey's multiple comparison test ((*) p<0.05, (**) p<0.01, (***) p<0.001, (****) p<0.0001).

FIG. 29. Immune responses after subcutaneous injection of PBS (negative control), compound 1, 1+CFA, 1+alum, 1+MF59, 1+ASO4 (adjuvanted controls) and 5-11 in C57BL/6 mice (n=5). (a) J8-specific serum IgG antibody titers and (b) J8-specific saliva IgG antibody titers, as analyzed by ELISA. Each point represents an individual mouse; bars represent the average antigen-specific IgG antibody titers. Statistical analysis was performed using one-way ANOVA with Tukey's multiple comparison test ((*) p<0.05, (**) p<0.01, (***) p<0.001, (****) p<0.0001). (c) Average opsonization percentage of different GAS strains (D3840 and GC2 203) by serum collected on day 49 following the primary immunization of C57BL/6 mice (n=5) with pHAA conjugates 5-11 and controls.

FIG. 30. Analysis of the purified Compounds 1-11 by analytical RP-HPLC (a) and ESI-MS (b). The compounds were purified using preparative RP-HPLC with solvent B concentration gradient 25-45% (1), 35-55% (9 and 11), 40-60% (7-8), 65-85% (2-5), and 80-100% (6 and 10) from Rₜ 5 to 30 min with C18 (1), C4 (2-9) or C8 (11) column. Analytical RP-HPLC graphs show pure compounds in single peak. The mass from these peaks, matched to the desired compounds in ESI-MS.

FIG. 31. DLS spectra of particles size by intensity (top) and number (bottom) for Compounds 1-11.

FIG. 32. Secondary structure of Compound 1, and 5-11 and the analysis of their α-helix, β-strand and random coil content.

FIG. 33. p145-specific serum IgG antibody titers after subcutaneous injection of PBS, Compound 1 (negative controls), 1+CFA, 1+alum, 1+MF59, 1+ASO4 (adjuvanted controls) and 5-11 in C57BL/6 mice (n=5), as analysed by ELISA. Each point represents individual mouse; a bar represents the average antigen-specific serum IgG antibody titers. Statistical analysis was performed using one-way ANOVA with Tukey's multiple comparison test ((*) p<0.05, (**) p<0.01, (***) p<0.001, (****) p<0.0001).

FIG. 34. Specificity of immune response. Sera (1:200 dilution) from mice (n=5) vaccinated with PBS, PADRE-88/30+CFA and 5 were used on ELISA plates coated with (Leu)₁₅-PADRE-88/30. Conjugate 5 did not induce antibody production against PADRE and polyleucine. Each point represents individual mouse; a bar represents the average antigen-specific serum IgG antibody optical density. Statistical analysis was performed using one-way ANOVA with Tukey's multiple comparison test (****; p<0.0001).

FIG. 35. Subcutaneous immunisation schedule with marked immunisation, serum and saliva collection days.

FIG. 36. Anti-RBD IgG responses following single subcutaneous immunization of BALB/c mice with RBD or epitopes adjuvanted with CFA presented as OD₄₅₀ values. The immunized group were compared with PBS and statistical analysis was performed using one-way ANOVA with Tukey's multiple comparison test; **p<0.01, ****p<0.0001.

FIG. 37. RBD/ACE2 binding inhibition assay in presence of A) B3 immunized mice serum, B) RBD immunized mice serum and C) mean group inhibition percent at various serum dilutions. Sera were serially diluted starting form 4-fold dilution.

FIG. 38. Anti-RBD IgG responses following three subcutaneous immunization with RBD or B3 adjuvanted with CFA or MF59 and vaccine candidates L1 and L2 presented as OD₄₅₀ values. Statistical analysis was performed using one-way ANOVA with Tukey's multiple comparison test; ****p<0.0001.

FIG. 39. RBD/ACE2 binding inhibition assay in presence of A) CFA+RBD, B) B3+PADRE+MF59, C) L1, D) L2, and E) PBS. Sera were serially diluted starting from 10-fold dilution.

FIG. 40. Analysis of the purified Compound (Leu)₁₀-B3 by analytical RP-HPLC (a) and ESI-MS (b). Analytical RP-HPLC graph shows the pure compound in single peak.

FIG. 41. Analysis of the purified Compound (LEU)₁₀-PADRE by analytical RP-HPLC (a) and ESI-MS (b). Analytical RP-HPLC graph shows the pure compound in single peak.

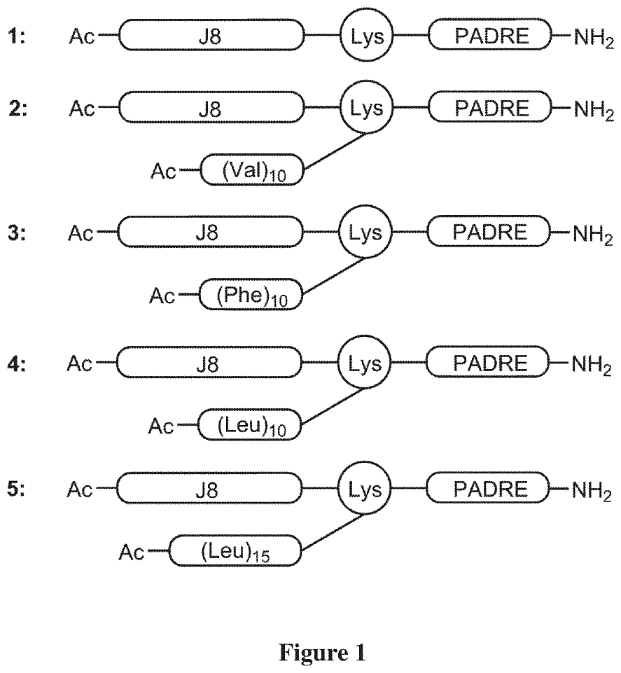

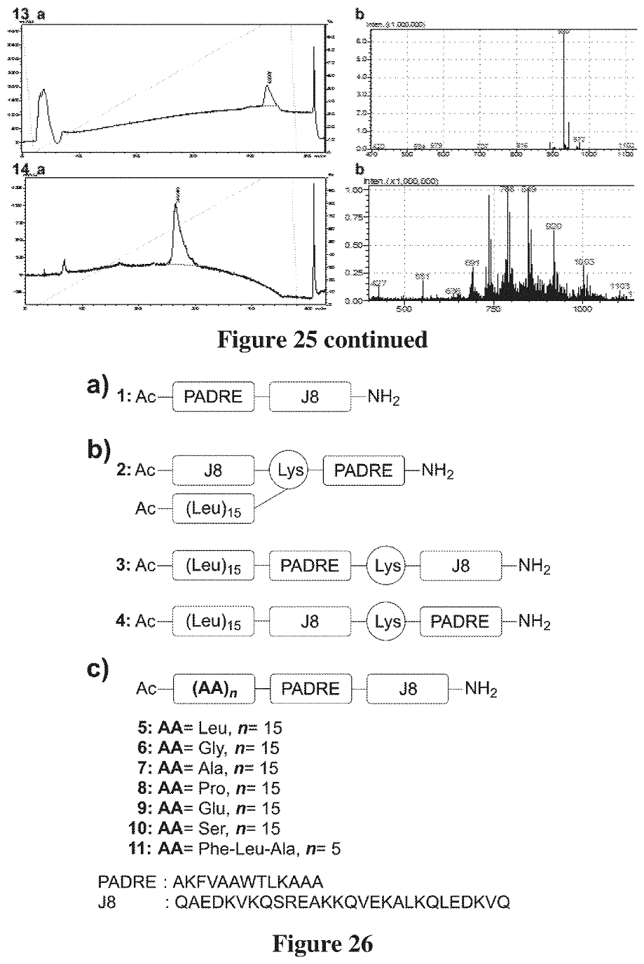

BRIEF DESCRIPTION OF THE SEQUENCES

| SEQ ID | | |
|---|---|---|
| NO: | Sequence | Description |
| 1 | LLLLLLLLLL | Synthetic polyleucine hydrophobic peptide. |
| 2 | LLLLLLLLLLLLLLL | Synthetic polyleucine hydrophobic peptide. |
| 3 | VVVVVVVVVV | Synthetic valine hydrophobic peptide. |
| 4 | FFFFFFFFFF | Synthetic phenylalanine hydrophobic peptide. |
| 5 | KKKKSS | Synthetic hydrophilic lysine and serine carrier or |
| | | linker. |
| 6 | SKKKK | Synthetic hydrophilic lysine and serine carrier or |
| | | linker. |
| 7 | AKFVAAWTLKAAA | Synthetic Pan-DR-helper cell epitope, PADRE. |
| 8 | KLIPNASLIENCTKAEL | Synthetic T helper epitope, P25. |
| 9 | QAEDKVKQSREAKKQVEKALKQLE | J8, a synthetic epitope derived from amino acids |
| | DKVQ | 344-355 of the M protein of M1 GAS strain |
| | | (shown in underline), flanked with GCN4 DNA- |
| | | binding protein sequences. |
| 10 | EVLTRRQSQDPKYVTQRIS | PL1, an epitope derived from type-specific N- |
| | | terminal from M protein of GAS strain M54. |
| 11 | DNGKAIYERARERALQELGP | 88/30, an epitope derived from type-specific N- |
| | | terminal from M protein of GAS strain 88/30. |
| 12 | TSLIAGPKAQVEAIQKYIGAEL | Necator americanus peptide p3 derived from Na- |
| | | APR-1, called A₂₉₁ Y. |
| 13 | RAHYNIVTF | E7₄₉₋₅₇ peptide, derived from HPV-16 E7 |
| | | oncoprotein. |
| 14 | QAEPDRAHYNIVTF | 8Qm (E744-57), an epitope derived from HPV-16 |
| | | E7 oncoprotein, see FIG. 14 b). |
| 15 | AIHADQLTPTWRVYSTG | SARS-CoV-2 epitope B1, S₆₂₃₋₆₃₉. |
| 16 | STEIYQAGSTPCNGVEGFNCYFPLQS | SARS-CoV-2 epitope B2, S₄₆₉₋₅₀₈. |
| | YGFQPTNGVGYQPY | |
| 17 | VGGNYNYLYRLFRKSNLKPFERDIST | SARS-CoV-2 epitope B3, S₄₄₅₋₄₈₃. |
| | EIYQAGSTPCNGV | |
| 18 | FLPFQQFGRDIADT | SARS-CoV-2 epitope B4, S₅₅₉₋₅₇₂. |
| 19 | SVLYNSASFSTFKCYGVSPTKLNDLC | SARS-CoV-2 epitope B5, S₃₆₆₋₃₉₅. |
| | FTNV | |
| 20 | QAEDKVKQSREAKKQVEKALKQLE | Synthetic peptide incorporating J8, lysine and |
| | DKVQKAKFVAAWTLKAAA | PADRE, see Compound 1 of FIG. 1. |
| 21 | AKFVAAWTLKAAAQAEDKVKQSRE | Synthetic peptide incorporating PADRE and J8, |
| | AKKQVEKALKQLEDKVQ | see Compound 4 of FIG. 22 and Compound 1 |
| | | of FIG. 26. |
| 22 | AKFVAAWTLKAAAEVLTRRQSQDP | Synthetic peptide incorporating PADRE and |
| | KYVTQRIS | PL1, see Compound 5 of FIG. 22. |
| 23 | AKFVAAWTLKAAADNGKAIYERAR | Synthetic peptide incorporating PADRE and |
| | ERALQELGP | 88/30, see Compound 6 of FIG. 22. |
| 24 | TSLIAGPKAQVEAIQKYIGAELKK | Synthetic peptide incorporating p3, lysine and |
| | LIPNASLIENCTKAEL | P25. |
| 25 | LLLLLLLLLLQAEPDRAHYNIVTF | Immunogenic agent, Compound Leu-8Qm, |
| | | incorporating polyleucine hydrophobic peptide |
| | | and 8Qm, see FIG. 14 b). |
| 26 | LLLLLLLLLLAKFVAAWTLKAAAQA | Immunogenic agent incorporating polyleucine |
| | EDKVKQSREAKKQVEKALKQLEDK | hydrophobic peptide, PADRE and J8, see |
| | VQ | Compound 7 of FIG. 22. |
| 27 | LLLLLLLLLLAKFVAAWTLKAAAEV | Immunogenic agent incorporating polyleucine |
| | LTRRQSQDPKYVTQRIS | hydrophobic peptide, PADRE and PL1, see |
| | | Compound 8 of FIG. 22. |
| 28 | LLLLLLLLLLAKFVAAWTLKAAADN | Immunogenic agent incorporating polyleucine |
| | GKAIYERARERALQELGP | hydrophobic peptide, PADRE and 88/30, see |
| | | Compound 9 of FIG. 22. |
| 29 | LLLLLLLLLLLLLLLAKFVAAWTLKA | Immunogenic agent incorporating polyleucine |
| | AAKQAEDKVKQSREAKKQVEKALK | hydrophobic peptide, PADRE, lysine and J8, see |
| | QLEDKVQ | Compound 3 of FIG. 26. |
| 30 | LLLLLLLLLLLLLLLQAEDKVKQSRE | Immunogenic agent incorporating polyleucine |
| | AKKQVEKALKQLEDKVQKAKFVAA | hydrophobic peptide, J8, lysine and PADRE, see |
| | WTLKAAA | Compound 4 of FIG. 26. |
| 31 | LLLLLLLLLLLLLLLAKFVAAWTLKA | Immunogenic agent incorporating polyleucine |
| | AAQAEDKVKQSREAKKQVEKALKQ | hydrophobic peptide, PADRE and J8, see |
| | LEDKVQ | Compound 5 of FIG. 26. |
| 32 | QQQQQQQQQQQQQQQAKFVAAWT | Immunogenic agent incorporating poly glutamine |
| | LKAAAQAEDKVKQSREAKKQVEKA | hydrophobic peptide, PADRE and J8, see |
| | LKQLEDKVQ | Compound 9 of FIG. 26. |
| 33 | GGGGGGGGGGGGGGGAKFVAAWT | Immunogenic agent incorporating polyglycine |
| | LKAAAQAEDKVKQSREAKKQVEKA | hydrophobic peptide, PADRE and J8, see |
| | LKQLEDKVQ | Compound 6 of FIG. 26. |
| 34 | SSSSSSSSSSSSSSSAKFVAAWTLKAA | Immunogenic agent incorporating polyserine |
| | AQAEDKVKQSREAKKQVEKALKQL | hydrophobic peptide, PADRE and J8, see |
| | EDKVQ | Compound 10 of FIG. 26. |
| 35 | PPPPPPPPPPPPPPPAKFVAAWTLKAA | Immunogenic agent incorporating polyproline |
| | AQAEDKVKQSREAKKQVEKALKQL | hydrophobic peptide, PADRE and J8, see |
| | EDKVQ | Compound 8 of FIG. 26. |
| 36 | AAAAAAAAAAAAAAAAKFVAAWT | Immunogenic agent incorporating polyalanine |
| | LKAAAQAEDKVKQSREAKKQVEKA | hydrophobic peptide, PADRE and J8, see |
| | LKQLEDKVQ | Compound 7 of FIG. 26. |
| 37 | FLAFLAFLAFLAFLAAKFVAAWTLK | Immunogenicagentincorporating |
| | AAAQAEDKVKQSREAKKQVEKALK | phenylalanine-leucine-alaninerepeat |
| | QLEDKVQ | hydrophobic peptide, PADRE and J8, see |
| | | Compound 11 of FIG. 26. |
| 38 | LLLLLLLLLLGVGGNYNYLYRLFRKS | Immunogenic agent, (Leu)₁₀-B3, incorporating |
| | NLKPFERDISTEIYQAGSTPCNGV | B3. |
| 39 | LLLLLLLLLLAKFVAAWTLKAAA | Immunogenicagent,(Leu)₁₀-PADRE, |
| | | incorporating PADRE. |
| 40 | LRRDLDASREAKKQVEKALE | pl45, derived from the M protein of M1 GAS |
| | | strain. |

Any or all N-terminal ends of the immunogenic agent can be acetylated or not. Any or all C-terminal ends of the immunogenic agent can be subjected to amidation or not. The sequences of the sequence listing can represent both modified and non-modified ends.

So that the invention may be fully understood and put into practical effect, reference is made to the following non-limiting Examples.

### EXAMPLES

### Example 1

Vaccines are one of the most powerful tools to combat infectious diseases. While whole pathogen and protein-based vaccines can provide very efficient protection against pathogens, they are not always entirely safe and may induce undesired immune responses. In such cases, peptide- and protein-based vaccines which are designed to induce only very specific immune responses against selected epitopes, are a natural alternative [1,2]. For instance, all the recent vaccines against Group A Streptococcus (GAS), which have entered clinical trials, are based on peptides derived from GAS major virulent factor, M-protein, but not on the protein itself [3]. GAS is responsible for variety of mild infection as well as for life-treating autoimmune diseases such as rheumatic fever (RF) and rheumatic heart disease (RHD) [4], which are estimated to kill 1.4 million people worldwide per year [5]. M-protein is anticipated to be the major antigen responsible for triggering these autoimmune responses [6]. Despite this, it is possible to identify M-protein-based peptide antigens which are safe and conserved among the vast number of GAS serotypes [7]. Such antigens need to be co-administered with adjuvant and/or an appropriate delivery system to induce any immune responses. However, the choice of safe and highly efficient adjuvants which are able to stimulate immune responses against peptide epitopes are limited [8]. In general, Alum is the only adjuvant widely used for formulation of commercial vaccines, while a few other adjuvants have been approved only for particular vaccine formulations. However, Alum is too poor an adjuvant to stimulate strong immune responses against peptides [9]. Powerful adjuvants, such as “gold standard” Complete Freund's Adjuvant (CFA) which is effective in triggering peptide antigen recognition by the immune system, are often toxic and contain poorly chemically defined fragments of bacteria or toxins [10]. Therefore, development of new adjuvants especially for weakly immunogenic antigens is a clear unmet clinical need [11].

Hydrophobic dendritic poly(tert-butyl acrylate) has been conjugated to a variety of peptides epitopes, including GAS-derived, and self-assembled conjugates to form particles [12]. The produced particles induced strong humoral and cellular immune responses against incorporated peptide antigens without any sign of adverse effects [12, 13, 14, 15, 16]. However, the lack of biodegradability, undefined stereochemistry and reproducibility are serious limitations in such adjuvant/vaccine compositions that limit the potential for successful commercial application.

In the present Example, we designed potent peptide-based vaccine delivery systems based on fully-defined and biodegradable polymers built from natural hydrophobic amino acids.

Materials and Methods

Reagents, cell and equipment. All chemicals were used as received, without any purification. Butyloxycarbonyl (Boc)-protected L-amino acids and was purchased from Novabiochem (Merck Chemicals, Darmstadt, Germany) and Mimotopes (Melbourne, Australia); methanol, trifluoroacetic acid (TFA), N,N′-diisopropylethylamine (DIPEA), N,N′-dimethylformamide (DMF), acetonitrile, and dichloromethane (DCM) were purchased from Merck (Hohenbrunn, Germany); 4-methylbenzhydrylamine (pMBHA) resin was purchased from Peptides International (Kentucky, USA); 2-(7-aza-1H-benzotriazole-1-yl)-1,1,3,3-tetramethyluronium hexafluorophosphate (HATU) was purchased from Mimotopes (Melbourne, Australia); p-cresol was obtained from Merck Millipore (Bayswater, Australia). Goat anti-mouse IgG (H+L)-HRP (IgG-HRP) conjugate was acquired from Millipore (Temecula, Calif., United States), goat anti-mouse IgA was obtained from Invivogen (San Diego, United States) and analytical-grade Tween 20, (tris-hydroxymethyl)aminomethane and glycine were acquired from VWR International (Queensland, Australia). Phenol-free IMDM Glutamax medium was purchased from Gibco (California, United States). Streptex™ Latex Agglutination Test kit and phenylmethylsulfonyl fluoride (PMSF) were purchased from Thermo Scientific (Victoria, Australia). PE/CY7 anti-mouse CD11C was purchased from eBioscience (California, United States), and BV421 anti-mouse MHC-II, FITC anti-mouse CD40 and BV605 anti-mouse F4/80 were purchased from BioLegend (California, United States). Fc-block was obtained from eBioscience (California, United States). Yeast extract was purchased from Merck Chemicals (Darmstadt, Germany). Todd-Hewitt broth (THB) was purchased from Oxoid (Thermo Fisher Scientific, South Australia, Australia) and horse blood was obtained from Serum australis. C57BL/6 mice were purchased from Animal Resources Centre (Western Australia, Australia). GAS strains 5448AP used in GAS intranasal challenge were obtained from Prof. Mark Walker's lab. GAS strain ACM-2002 was obtained from Royal Brisbane hospital (human abscess-lymph gland), ACM-5199 (ATCC 12344, NCIB 11841, scarlet fever), ACM-5203 (ATCC 19615, pharynx of child followed by sore throat), GC2 203 (wound swab), D3840 (naso-pharynx swabs) and D2612 (naso-pharynx swabs). All other chemicals were purchased from Sigma-Aldrich (Victoria, Australia).

Analytical RP-HPLC was performed on Shimadzu (Kyoto, Japan) instrument with 1 mL/min flow rate with compound detection at 214 nm. The preparative RP-HPLC was achieved with Shimadzu (Kyoto, Japan) instrumentation (either LC-20AT, SIL-10A, CBM-20A, SPD-20AV, FRC-10A or LC-20AP×2, CBM-20A, SPD-20A, FRC-10A) in linear gradient mode. The flow rate was 10 or 20 mL/min and the compounds were detected at 230 nm. Separations were achieved with solvent A (100% H₂O and 0.1% TFA) and solvent B (90% acetonitrile, 10% H₂O and 0.1% TFA) on Vydac 214TP1022 preparative column (C4, 10 m, 22 mm×250 mm). A Perkin-Elmer-Sciex API3000 instrument with Analyst 1.4 software (Applied Biosystems/MDS Sciex, Toronto, Canada) was used for electrospray ionization mass spectrometry (ESI-MS). Particle size and morphology were analyzed by DLS using a Nanosizer (Zetasizer Nano Series ZS, Malvern Instruments, United Kingdom) with disposable capillary cuvettes using Dispersion Technology Software (Malvern Instruments, United Kingdom) and by TEM using a JEM-1010 microscope (JEOL Ltd., Japan). Secondary structures of Compounds 1-5 were analyzed by a circular dichroism (CD) instrument (Jasco J710 spectropolarimeter, JASCO, Japan) with a 1 mm cell (Starna) at RT. The absorbance of each well in cytotoxicity analysis was measured at 580 nm with a PowerWave XS Microplate Reader from Bio-Tek Instruments Inc. (Winooski, Vt.). A LSR II flow cytometry instrument (LSR II Flow cytometer, BD Biosciences, California, United States) was applied in APC maturation study. A RS-VA 10 vortex mixer (Phoenix Instrument, Germany) was used for vaccination solution preparation. ELISA plates were analyzed in a Victor3 1420 multi-label counter (Perkin Elmer Life and Analytical Sciences, Shelton, United States).

Synthesis and purification of pHAA-peptide conjugates. Peptides 1-5 (FIG. 1) were synthesized by microwave-assisted standard Boc-solid-phase peptide synthesis (SPPS) method [17]. Briefly, deprotection of Boc group was performed twice (1×2 min and 1×3 min) with neat TFA, and double coupling of amino acids were applied (1×5 min and 1×10 min, 20 W, 70° C.). Amino acids were activated by DIPEA and HATU. Peptides were cleaved by anhydrous hydrogen fluoride (HF) with addition of p-cresol as a scavenger. Compounds 1-5 were purified by RP-HPLC.

Compound 1. Yield: 34%. Molecular Weight: 4823.63. ESI-MS [M+3H]3+ m/z 1608.6 (calc. 1608.9), [M+4H]⁴⁺ m/z 1206.8 (calc. 1206.9), [M+5H]⁵⁺ m/z 965.8 (calc. 965.7), [M+6H]⁶⁺ m/z 804.9 (calc. 804.9), [M+7H]⁷⁺ m/z 690.1 (calc. 690.1), [M+8H]⁸⁺ m/z 603.9 (calc. 604.0), [M+9H]⁹⁺ m/z 537.0 (calc. 537.0). t_{R}=19.1 min (0-100% Solvent B; C4 column), purity ≥99%.

Compound 2. Yield: 23%. Molecular Weight: 5814.96. ESI-MS [M+3H]³⁺ m/z 1938.6 (calc. 1939.3), [M+4H]⁴⁺ m/z 1455.0 (calc. 1454.7), [M+5H]⁵⁺ m/z 1164.0 (calc. 1164.0), [M+6H]⁶⁺ m/z 969.9 (calc. 970.1), [M+7H]⁷⁺ m/z 831.5 (calc. 831.7), [M+8H]⁸⁺ m/z 728.0 (calc. 727.9). t_{R}=24.1 min (0-100% Solvent B; C4 column), purity ≥99%.

Compound 3. Yield: 28%. Molecular Weight: 6295.40. ESI-MS [M+4H]⁴⁺ m/z 1574.7 (calc. 1574.9), [M+5H]⁵⁺ m/z 1260.0 (calc. 1260.0), [M+6H]⁶⁺ m/z 1050.3 (calc. 1050.2), [M+7H]⁷⁺ m/z 900.4 (calc. 900.3), [M+8H]⁸⁺ m/z 788.0 (calc. 787.9), [M+9H]⁹⁺ m/z 700.7 (calc. 700.5). t_{R}=24.5 min (0-100% Solvent B; C4 column), purity ≥99%.

Compound 4. Yield: 28%. Molecular Weight: 5955.23. ESI-MS [M+3H]³⁺ m/z 1985.2 (calc. 1986.1), [M+4H]⁴⁺ m/z 1489.6 (calc. 1489.8), [M+5H]⁵⁺ m/z 1192.0 (calc. 1192.0), [M+6H]⁶⁺ m/z 993.5 (calc. 993.5), [M+7H]⁷⁺ m/z 851.8 (calc. 851.7), [M+8H]⁸⁺ m/z 745.4 (calc. 745.4). t_{R}=25.6 min (0-100% Solvent B; C4 column), purity ≥99%.

Compound 5. Yield: 26%. Molecular Weight: 6521.03. ESI-MS [M+4H]⁴⁺ m/z 1631.8 (calc. 1631.3), [M+5H]⁵⁺ m/z 1305.6 (calc. 1305.2), [M+6H]⁶⁺ m/z 1088.0 (calc. 1087.8), [M+7H]⁷⁺ m/z 932.9 (calc. 932.6), [M+8H]⁸⁺ m/z 816.3 (calc. 816.1), [M+9H]⁹⁺ m/z 725.6 (calc. 725.6). t_{R}=30.9 min (0-100% Solvent B; C4 column), purity ≥99%.

Particle size measurements. Compounds 2-5 were self-assembled in PBS to prepare 0.5 mg/mL solution. Then the average particle sizes (nm) of the produced nanostructures were measured by DLS at 25° C. Sizes were analyzed using a non-invasive backscatter method and measurements were taken with a 1730 scattering angle. Correlation times were based on 10 seconds per run and at least ten consecutive runs were made per measurement. Each measurement was repeated five times. The same compound solutions were also analyzed by TEM.

Secondary structure analyses. The secondary structure of Compounds 1-5 (0.1 mg/mL in PBS) were analyzed by CD. Spectra were tested with the following parameters: 5 nm bandwidth; scan rate 50 nm/min; response time two seconds; and 1 nm intervals over the wavelength range 195-260 nm in a nitrogen atmosphere. Reported data are the mean of six accumulations. Mean residue molar ellipticity (deg·cm²·dmol⁻¹) was calculated using the formula [θ]=mdeg/(l×c×n)*1000, where: l is path length (0.1 cm); c is peptide concentration (mM); and n is the number of residues in the peptide.

Ethics statement. All animal protocols used were approved by the Griffith University Animal Ethics Committee, GU Ref No: GLY/07/14. This study was carried out in accordance with the NHMRC of Australia guidelines for generating, breeding, caring for and using genetically modified and cloned animals for scientific purposes (2007). Methods were chosen to minimize pain and distress to the mice, which were observed daily by trained animal care staff. The mice were terminated using a C02 inhalation chamber.

Cytotoxicity results study. The 3-(4,5-dimethylthiazol-2-yl)-2,5-diphenyltetrazolium bromide (MTT) cytotoxicity assay was performed using SW620 and HEK293 adherent cell lines. SW620 and HEK293 were cultured in RPMI-1640 medium and Dulbecco's modified Eagle medium (DMEM), respectively, as adherent mono-layers in flasks supplemented with 10% fetal bovine serum, 2 mM L-glutamine, 100 unit/mL penicillin and 100 μg/mL streptomycin in a humidified 37° C. incubator supplied with 5% C02. Cells were then harvested with trypsin and dispensed into 96-well microtitre assay plates at 2,000 cells/well for the two cell lines, and incubated for 18 hours at 37° C. with 5% C02 (to allow cells to attach). Compounds 2-5 were dissolved in 5% DMSO in PBS (v/v) and aliquots (10 μL) tested over a series of final concentrations ranging from 10 nM to 30 μM. Control wells were treated with 5% aqueous DMSO. After a 68-hour incubation at 37° C. with 5% C02, an aliquot (10 μL) of MTT in PBS (5 mg/mL) was added to each well (final concentration of 0.5 mg/mL), and the microtitre plates were incubated for a further four hours at 37° C. with 5% C02. After this final incubation, the medium was aspirated and precipitated formazan crystals dissolved in DMSO (100 μL/well). Then the absorbance of each well was measured. Vinblastine was used as a positive control (20 mg/mL in H₂O). All experiments were performed in duplicate. Half maximal inhibitory concentration (IC50) values were calculated using GraphPad Prism® 7 software (GraphPad Software, Inc., California, United States).

Antigen-presenting cells maturation study. Single-cell spleen suspensions were harvested from Swiss naive mice by physically disruption of their spleens and passing the disrupted spleens through stainless-steel mesh. Red blood cell lysis buffer was used to lyse the erythrocytes. Then, a 96-well plate was filled with 2×10⁵ cells/well in phenol-free IMDM Glutamax medium supplemented with 10% fetal bovine serum, 50 mM 2-mercapto-ethanol, 100 U/mL penicillin and 100 mg/mL streptomycin. For each well of the plates, 10 μM of Compounds 2-5 was added, and the plates were incubated at 37° C. for six hours. Cells that adhered to the wells were scraped first, and then Fc-block was added. Following this, the plates were put in an incubator for 30 minutes at 4° C. The cells were centrifuged and resuspended in a buffer containing CD11c, F4/80, CD40, CD80 and MHC-II antibodies for 30 min at 4° C. After the incubation, the plates were centrifuged and washed again. The cells were then resuspended in 0.5 mL of FACS buffer (PBS, 0.02% sodium azide, 0.5% BSA). Both percentage fluorescence positive and mean fluorescence intensity for CD11C or F4/80 cells and activation markers CD40, CD80 and MHC-II were used to identify the maturation of dendritic cells or macrophages.

Vaccination and bacterial infection. C57BL/6 female mice received primary immunization and three boosts with Compounds 2-5 on days 0, 21, 28 and 35. The Compounds 2-5/PBS solutions and Compound 1/CFA emulsion were prepared freshly before each immunization. Compounds 2-5 were dissolved in PBS directly and then vortexed for two minutes. Mice in each group (10 mice/group) were immunized subcutaneously at the base of their tails with 150 μg of compound in 50-100 μL of PBS.

All immunized and control mice were challenged intranasally by the GAS strain M1 with a predetermined dose on the 61st day post primary immunization. The throat swab was obtained on days 1-3 after the mice were challenged with the GAS bacteria. Columbia base agar plates containing 2% defibrinated horse blood were prepared first. All throat swabs were streaked on these plates and incubated at 37° C. The plates were stored in 4° C. cooling room for later determination of GAS colonization. Nasal shedding was determined on days 1-3 post-challenge by pressing the nares of each mouse onto the surface of the prepared Columbia blood agar (CBA) plates 10 times (triplicate CBA plates/mouse/day) and exhaled particles were streaked out. On days two and three post challenge, five mice from each group were sacrificed to harvest the nasal-associated lymphoid tissue (NALT) and spleen sample. The NALT and spleen samples were homogenized in PBS first, and then plated in serial dilutions on Columbia base agar plates containing 2% defibrinated horse blood to assess bacterial burden.

Evaluation of antibody titers by enzyme-linked immunosorbent assay (ELISA). The J8-specific IgG in murine serum/saliva and J8-specific IgA in murine saliva collected from the immunized C57BL/6 mice after each immunization were measured by ELISA. Polycarbonate plates were coated by J8 peptide (pH 9.6, 0.5 mg/mL in a carbonate coating buffer) with 100 μL/well amount, and incubated at 4° C. overnight. Then after washing the plates five times with PBS-Tween 20 buffer; 150 μL of 5% skim milk PBS-Tween 20 was added. After incubation at 37° C. for two hours, plates were washed again. Sera samples (200 μL of 1:100 dilution) or saliva samples (100 μL of 1:2 dilution) were added to the plate followed by serial dilution down the plate in 0.5% skim milk PBS-Tween 20 buffer. All plates were then incubated for 1.5 h in a 37° C. incubator. The plates were washed five times and 100 μL/well 1:3000 diluted peroxidase-conjugated goat anti-mouse IgG (in 0.5% skim milk PBS-Tween 20) or 50 μL/well 1:1000 diluted goat anti-mouse IgA (in 0.5% skim milk PBS-Tween 20) were added, and the plates were incubated 1.5 h at 37° C. After incubation, plates were washed again, and 100 μL/well OPD substrate was added. The plates were incubated for 30 minutes in a dark environment at room temperature, and then the absorbance of stained J8-specific IgG or IgA was measured at λ 450 nm.

Bactericidal assessment. Opsonization assay was performed with serum collected from immunized mice to evaluate the bactericidal efficacy. The following clinical isolates were examined: ACM-2002, ACM-5199, ACM-5203, GC2203, D3840, D2612. The bacteria were prepared first-they were streaked on a THB supplemented with 5% yeast extract agar plate. The plate was then placed in a 37° C. incubator for 24 hours. Following this, one single colony obtained from the bacterium was transferred to THB (5 mL) supplemented with 5% yeast extract, and the plate was then incubated for 24 hours at 37° C. When the bacteria duplicated to approximately 4.6×106 cc/ml, the culture was serially diluted to 10⁻² in PBS and an aliquot (10 μL) was mixed with heat-inactivated tested sera (10 μL) collected on the 49th day post primary immunization from immunized mice and horse blood (80 μL). The inactivated sera were prepared by being heated in a 50° C. water bath for 30 minutes. The bacteria incubation was conducted in the presence of sera in a 96-well plate at 37° C. for three hours. Then plated 10 μL culture material on Todd-Hewitt agar plates supplemented with 5% yeast extract and 5% horse blood. The plates were incubated at 37° C. for 24 hours. The bacteria survival rate was analyzed based on the colony forming units (CFU) enumerated from the incubated Todd-Hewitt agar plates. The assay was performed in triplicate from three independent cultures.

Statistical analysis. GraphPad Prism® 7 software was used for all statistical analysis. For the APC maturation results, mean fluorescence intensity were recorded. Two-way ANOVA followed by Tukey's multiple comparison test was applied during the statistical analysis using GraphPad Prism® 7 software (GraphPad Software, Inc., California, United States), with p<0.05 considered statistically significant. The titer of the J8-specific IgG or IgA was described as the lowest dilution that offered an absorbance of > three SD above the mean absorbance of negative control wells (wells coated with serum from mice immunised with PBS). One-way ANOVA followed by the Tukey's post hoc test was applied for the antibody titers statistical analysis, with p<0.05 considered statistically significant. The opsonic activity of the antibodies (anti-peptide) sera (% reduction in mean CFU) was calculated as [1-(CFU in the presence of anti-peptide sera)/(mean CFU in the presence of PBS)]×100. Two-way ANOVA followed by the Tukey's post hoc test was applied for the opsonization statistical analysis, with p<0.05 considered statistically significant. The bacteria infection challenge evaluation of CFU was recorded and analyzed by two-way ANOVA followed by the Tukey's post hoc test was applied, with p<0.05 considered statistically significant.

Results

Synthesis of pHAA-peptide conjugates. The B-cell epitope J8 and T-helper PADRE have been conjugated on the resin using standard Boc-SPPS method [17], with lysine as a spacer and branching moiety for attachment of pHAA, to produce peptide 1 (Figure. 1). Peptide 1 was modified with ten units of valine, ten units of phenylalanine, ten units of leucine, or fifteen units of leucine, to yield Compounds 2, 3, 4, 5 respectively. All compounds were acetylated on their N-termini. Compound 5, bearing fifteen leucines was successfully produced but precipitated above 1.5 mg/mL concentration when dissolved in water.

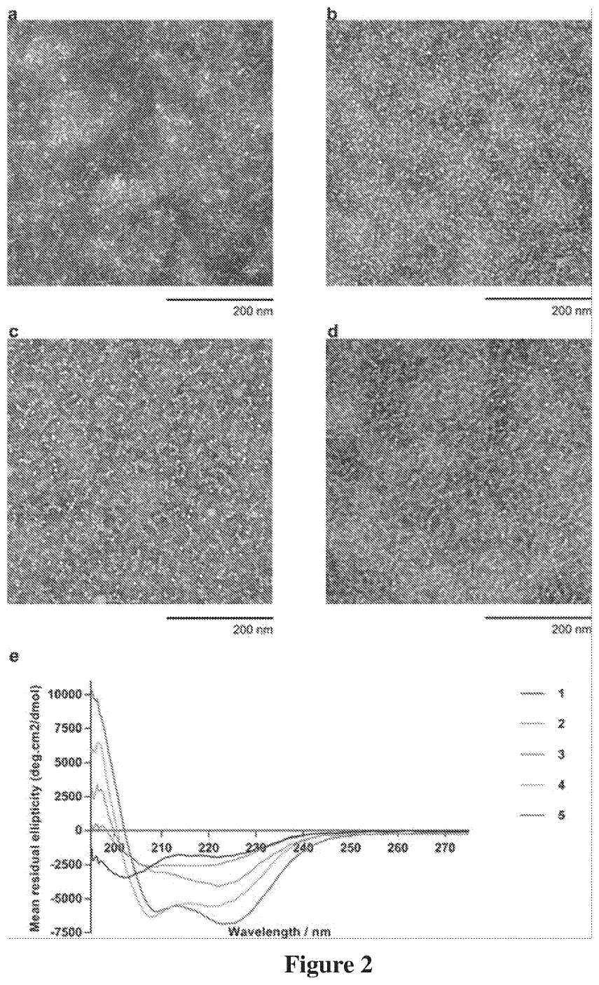

Self-assembly and characterization of pHAA-peptide nanoparticles. Hydrophilic peptide epitope 1 upon conjugation with hydrophobic pHAA sequences form conjugates which have amphiphilic properties and therefore are able to self-assemble to form particles. Thus, the Compounds (2-5) self-assembled in phosphate-buffered saline (PBS) as examined by dynamic light scattering (DLS) and transmission electron microscopy (TEM). All compounds self-assembled into mixtures of small nanoparticles (10-30 nm) and bigger aggregates with high polydispersity index according to DLS (Table 1 and FIG. 6) and TEM (FIG. 2a-d). On TEM images, distinct nanoparticles as well as chain-like aggregates of the nanoparticles (CLAN) are clearly visible (FIG. 2a), especially for Compound 5 (not shown).

TABLE 1
Particle size distribution for Compounds 2-5 analyzed by intensity.

| | 2 | 3 | 4 | 5 |
|---|---|---|---|---|
| pHAA | (Val)₁₀ | (Phe)₁₀ | (Leu)₁₀ | (Leu)₁₅ |
| size (nm) | 14 ± 1 | 28 ± 4 | 25 ± 3 | 300 ± 40 |
| | 150 ± 60 | 220 ± 20 | 170 ± 40 | 880 ± 100 |
| | 4840 ± 400 | 4380 ± 290 | 4520 ± 750 | 5390 ± 20 |
| PDI | 0.33 ± 0.05 | 0.45 ± 0.10 | 0.49 ± 0.05 | 0.50 ± 0.05 |

Cytotoxicity of conjugates 2-5. As proposed here, vaccine delivery system/adjuvant was constructed fully based on natural amino acids and we did not expect any toxicity of pHAA conjugates. Indeed, all compounds did not show any cytotoxicity against SW620 (human colon carcinoma) and HEK293 (human embryonic kidney cell line) at concentrations up to 30 μM.

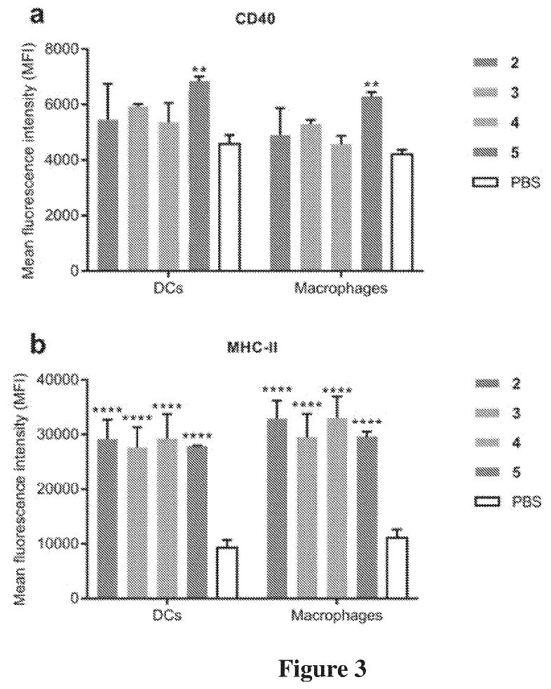

Ability of conjugates to maturate dendritic cells (DCs) and macrophages. Maturation of APCs (CD11c+ DCs and F4/80+ macrophages from murine splenocytes) was determined upon their treatment with 2-5 following analysis of the expression of MHC-II and the CD40 co-stimulatory molecule in vitro (FIG. 3). All conjugates induced higher expression of MHC-II compared to negative control, while only Compound 5 induced significantly higher expression of CD40 in both DCs and macrophages (p=0.011 and 0.0027 respectively).

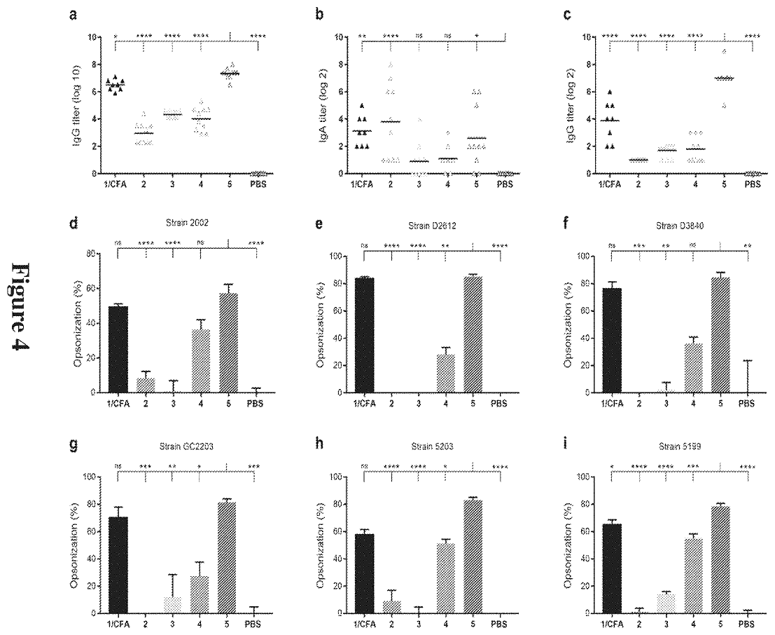

Systemic and mucosal immune responses induced by Compounds 2-5. C57BL/6 female mice received tail base subcutaneous immunization and three boosts with Compounds 2-5 on day 0, 21, 28 and 35. All compounds elicited a significant level of J8-specific IgG titers after the final immunization (FIG. 4a). Compound 5 induced significantly stronger responses than all other compounds including 1 emulsified with Freund's adjuvant (CFA), (p=0.0219).

Ability of the Compounds 1-5 to induce mucosal immune responses upon subcutaneous immunization was also examined. Conjugates 2 and 5 as well as peptide 1 emulsified with CFA were able to induce production of J8-specific IgA in mice (FIG. 4b). The IgA titers were relatively low, yet, significantly higher than those observed in mice treated with PBS. Similarly, the salivary IgG titers induced by immunization with conjugates (FIG. 4c) were very low (2), low (3, 4) or moderated (1/CFA) with the exception of Compound 5 which induce salivary IgG production significantly higher (p<0.0001) than that of positive control group (1/CFA).

The quality of produced antibodies was tested in an opsonization assay against a variety of GAS clinical isolates (FIG. 4d-i). Sera collected from mice immunized with compounds bearing leucine-based pHAA units (4 and 5) and 1/CFA induced high levels of opsonization of all GAS strains when compared with sera derived from the mice treated with PBS. Especially, the sera collected from mice immunized with Compound 5 demonstrated, in most cases, significantly better ability to kill bacteria than that from other groups (FIG. 4d-i), including 1/CFA (FIG. 4i). Compound 4, which was less potent than 5, still generated better opsonic responses than 2 and 3.

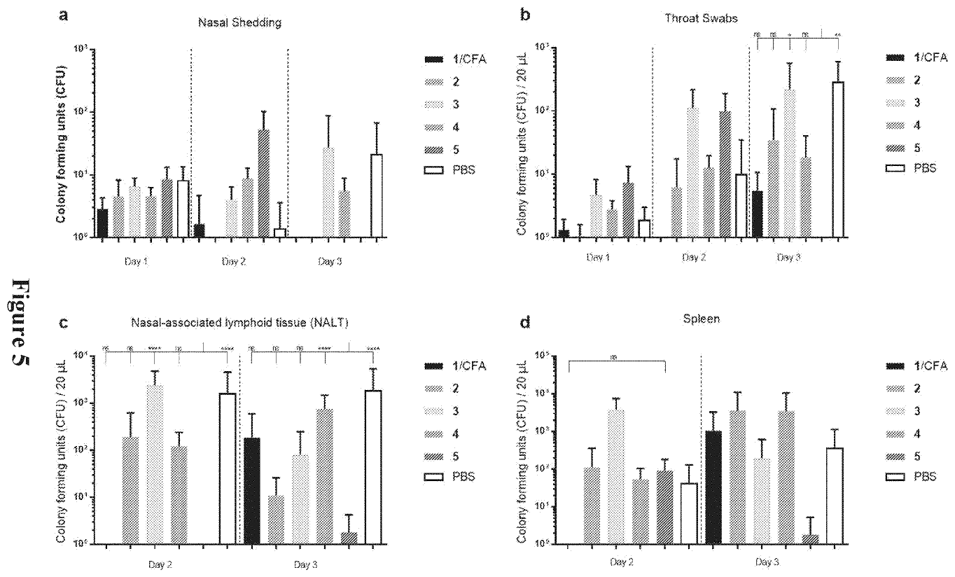

Post-immunization intranasal challenge with M1 GAS bacteria. Following intranasal bacteria challenge, the level of bacteria was assessed in nasal shedding, throat swabs, Nasal Associated Lymphoid Tissue (NALT; a murine functional homolog to human tonsils) [24] and spleen (FIG. 5). Both nasal shedding and throat swabs were analyzed using ten mice groups at day 1, 2 and 3. In both mucosal areas, Compound 5 demonstrated the highest efficacy in clearance of GAS bacteria which was practically not detectable in mice immunized with this conjugate on day 3 (FIG. 5a,b). In both these organs, similarly to mucosal area surfaces, 5 induced the strongest reduction of bacteria burden on the day 3 than any other compounds, including CFA adjuvanted peptide 1.

Discussion

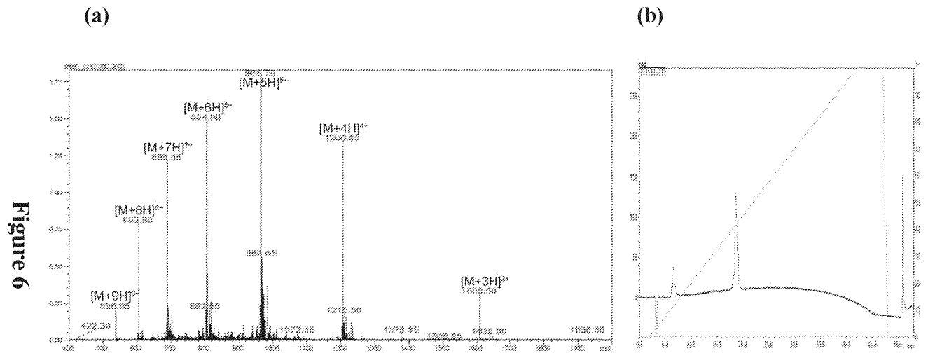

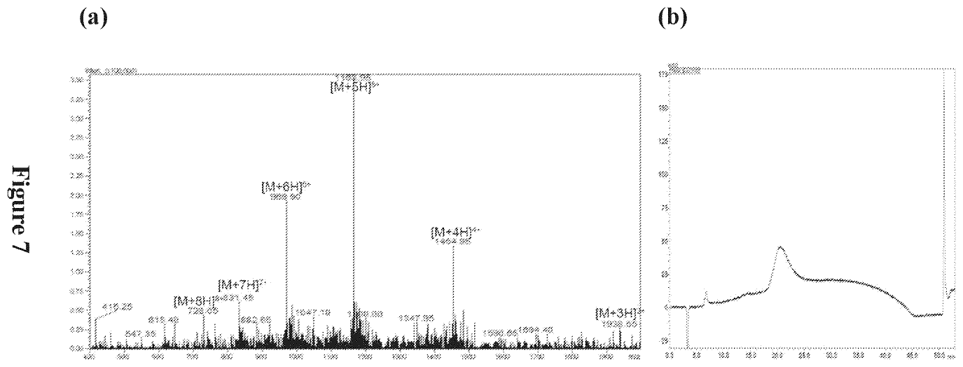

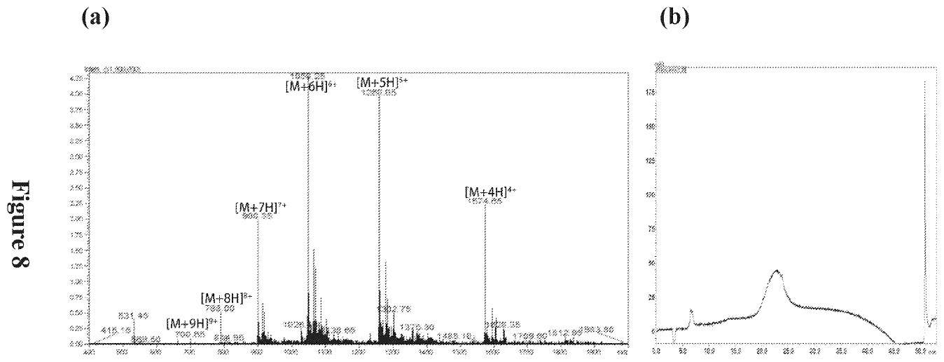

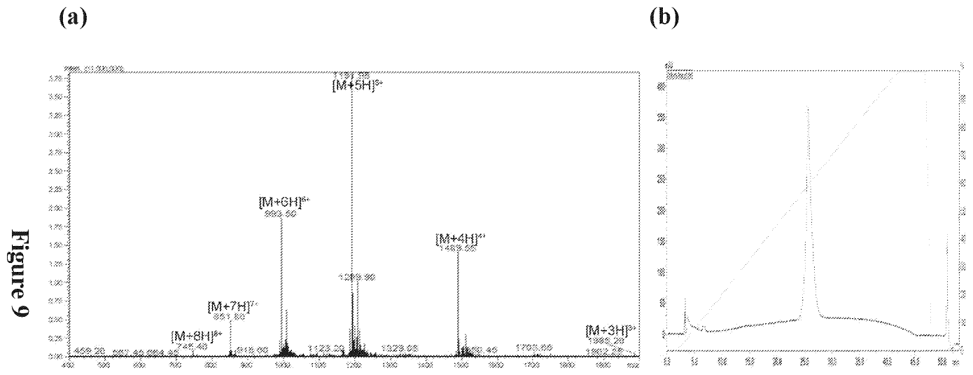

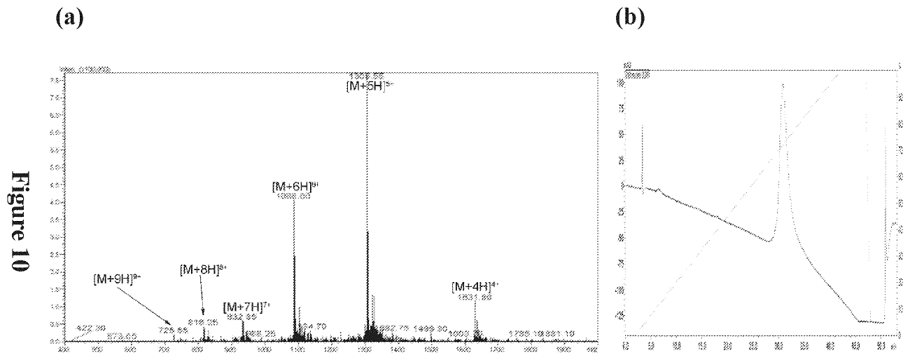

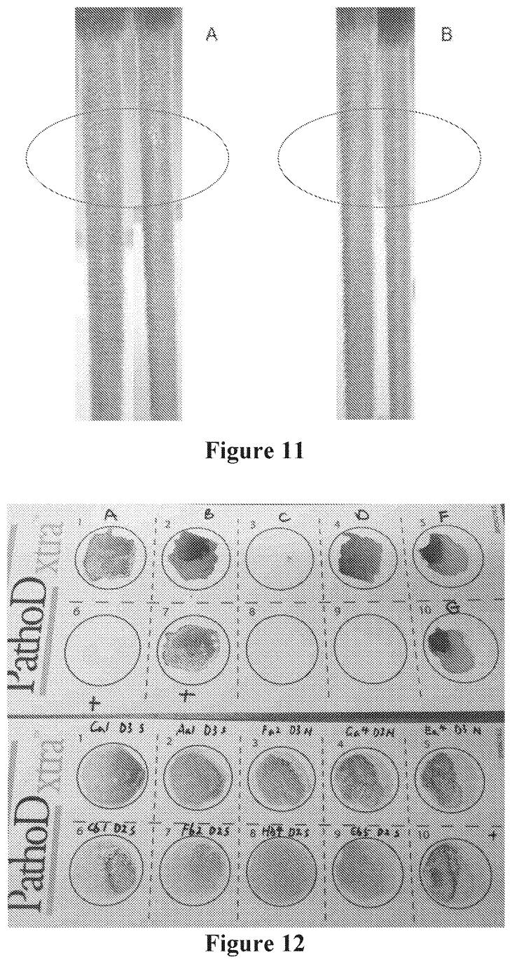

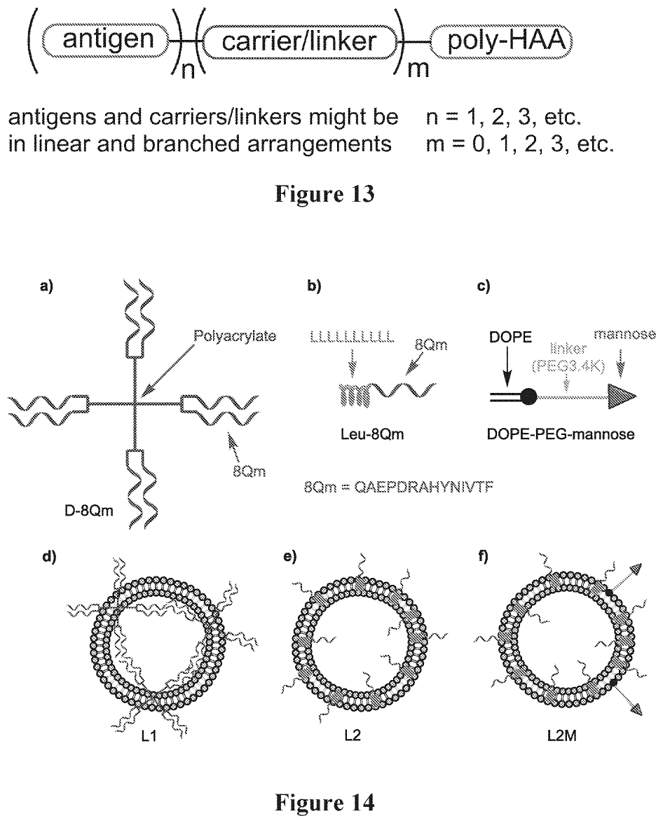

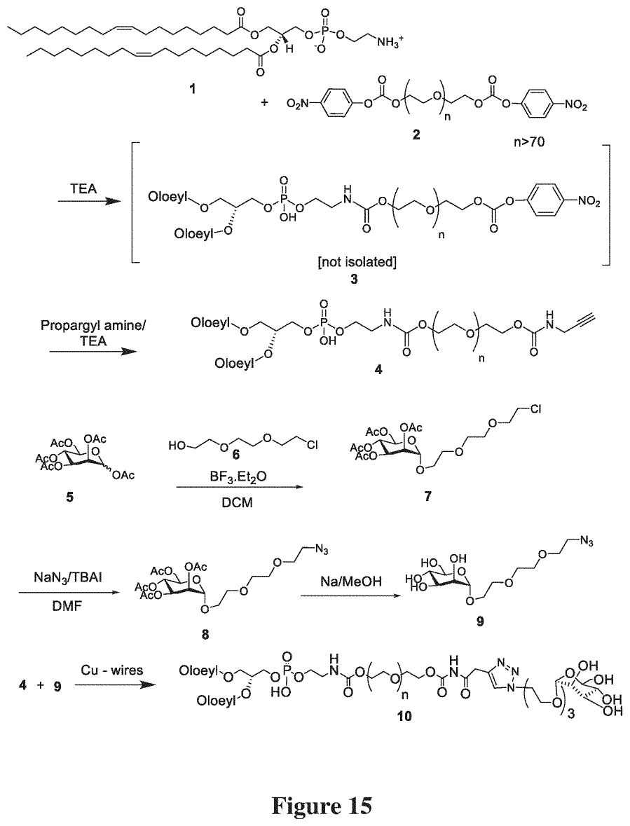

Adjuvants play a key role in modern vaccines [11]. Despite some safety concerns, many currently available adjuvants are not fully defined and may include mixtures of lipids, polysaccharides, polymers and various microbial components. Thus, a chemically-defined single compound that can help stimulate an immune response against the antigen it carries would be beneficial over the existing adjuvants. As nanoparticles are known to have promising self-adjuvanting properties [25], amphiphiles that self-assemble are often used in peptide vaccine delivery [26, 27]. Herein we propose a universal peptide-based platform comprising pHAA which upon conjugation to an antigen stimulates formation of nanoparticles. The properties of the pHAA unit (e.g. water-solubility and conformational, etc.) can be altered easily by changing its length and amino acid composition. We selected three hydrophobic amino acids (valine, phenylalanine, and leucine) to examine whether their polymeric arrangement when conjugated to a peptide epitope could self-assemble into nanoparticles. GAS B-cell epitope J8 conjugated to universal T-helper epitope PADRE (1) was selected as a vaccine antigen and modified with ten or fifteen copies of HAA (FIG. 1). pHAAs with the aforementioned lengths were expected to induce conjugate aggregation under aqueous conditions. Compounds that bore fifteen repeats of phenylalanine and valine were poorly water-soluble and were therefore excluded from further studies. Although the compound that bore fifteen leucines precipitated when the concentration was higher than 1.5 mg/mL, it was still practical for immunological evaluation. Thus, the maximum length of pHAA sequence which could be used to deliver peptide antigens was essentially limited by the compounds' aqueous solubility. Other Compounds 2-5 were easily water-soluble; however, as they possessed amphiphilic properties due to the presence of highly hydrophobic pHAA units and hydrophilic peptide antigen (1), they promptly self-assembled under aqueous conditions. All of Compounds 2-5 formed small nanoparticles and chain-like aggregates of nanoparticles (CLAN). Similar aggregates were previously reported for inorganic nanoparticles [28, 29]. The aggregative potential of all compounds were generally similar with the exception of Compound 5 which had a higher tendency to form CLAN (FIG. 2a-d, Table 1, FIG. 7) due to its hydrophobic nature. Interestingly, the same compound adopted the most distinct secondary structure. A classical double-minimum α-helix CD spectrum was observed for 5, while the spectra of its shorter analogue 4 and peptide antigen 1 showed a shift of the helical curve toward random coil (FIG. 2e). In contrast, Compounds 2 and 3 showed tendency to form a β-sheet. The antibodies induced against the J8 epitope need to recognize the parent helical M-protein, thus the conformational properties of the vaccine have an important role in its antibacterial efficacy. Indeed, following immunization with 5, antibodies generated by mice showed the strongest ability to opsonize GAS clinical isolates in vitro (FIG. 4.). At the same time, 3-sheet-rich Compounds 2 and 3 appeared to induce production of non-protective antibodies that were not able to kill GAS bacteria. Compound 5 also demonstrated excellent ability to protect mice in the M1 GAS challenge experiment (FIG. 5). Clearance of GAS bacteria was not observed in mice immunized with 4, which suggested that not only the opsonic ability but the quantity of antibodies played an important role in protection. Compound 5 induced significantly higher J8-specific systemic and mucosal IgG titers than the other compounds. Relatively low IgA titers were observed (as expected) after non-mucosal immunization and therefore the level of IgA production was not influential on the vaccine performance of the candidates. As antigen presentation and maturation of APCs is important for induction of effective humoral immunity, expression of MHC-II and CD40 was examined. MHC-II is an APC receptor required for peptide antigen presentation to Th cells and therefore the induction of adaptive immunity [30], while the level of CD40 expression is typically used as a marker for DC maturation in mice [31]. As expected all compounds stimulated overexpression of both proteins. However, statistically significant overexpression of CD40 was observed only in mice treated with Compound 5. Thus, Compound 5 clearly demonstrates the most promising properties among all examined pHAA-based immunogens.

While the quality of immune response elicited by Compound 5 can be explained by its ability to adopt a helical conformation due to the presence of a poly-leucine unit, the magnitude of response (e.g. high antibody titers) could not be explained by the conformation of the epitope and instead resulted from the higher hydrophobicity of the compound. When gold nanoparticles of the same size and charge were examined, those modified with more hydrophobic groups induced stronger immune system activation [32]. Importantly, the hydrophobic properties and conformation can easily be modified by changing the type and number of amino acids in the pHHA unit and therefore the system can easily be customized for different epitopes. Moreover, in comparison to many other self-assembling systems, an external adjuvant was not needed to trigger a strong immune response [33].

Throughout this specification, the aim has been to describe the preferred embodiments of the invention without limiting the invention to any one embodiment or specific collection of features. Various changes and modifications may be made to the embodiments described and illustrated herein without departing from the broad spirit and scope of the invention.

All computer programs, algorithms, patent and scientific literature referred to herein is incorporated herein by reference in their entirety.

### Example 2

Half a million people annually are diagnosed with cervical cancer triggered by human papilloma virus (HPV) infection, with deaths reaching over a quarter million in 2018 alone [1]. Over the past few decades, cases of HPV have risen, particularly in developing countries. While early identification using comprehensive cervical screening programs have decrease prevalence of cervical cancer, economics in developing countries are not sufficient enough to provide the resources needed for these screenings. Current treatment available against cervical cancer include chemotherapy, radiotherapy and surgery. However, treatment has a high failure rate due to high relapse in disease and development of drug resistance. A prophylactic vaccine has been developed against HPV infections. This vaccine should decrease the overall rate of cervical cancer; however, this will more than likely take more than 20 years to have an effect on the population [2]. As a large proportion of the world are already infected with HPV, the risk of these people developing cervical cancer is still high. Therefore, the development of a therapeutic vaccine which can target HPV-infected cells is important to treat cervical cancer.

Vaccines designed to treat cancer need to stimulate cytotoxic T lymphocyte (CTL) responses to eliminate abnormal cells bearing specific tumour antigens. Use of the whole HPV or HPV oncoprotein as antigen can lead to oncogenic changes and therefore a development of a peptide-based vaccine has been suggested [3]. A HPV oncoprotein called E7 is found in HPV infected cells and is responsible for maintaining HPV-associated tumour cell growth. Identification and development of vaccine candidates based on the E7 protein, specifically CD8+ peptides, can activate cytotoxic T lymphocytes, which can in turn destroy the tumour cells. 8Qm (E7_{44_57}, QAEPDRAHYNIVTF; SEQ ID NO:14), is an epitope derived from HPV-16 E7 oncoprotein which has shown to have a therapeutic effect against cervical cancer in mice [4]. It bears both CD4+ and CD8+ epitopes. Alone however, peptides such as 8Qm are not able to stimulate an immune response due to their poor immunogenicity and low stability in vivo.

Incorporation of the peptides into a delivery system or co-administered with adjuvants is required to produce an effective vaccine. Typical problems associated with the use of currently available adjuvants include toxicity, low efficacy and a limited choice of adjuvants appropriate for human use [5, 6]. Therefore, developing novel adjuvants (or delivery systems) with potent immunomodulatory activities on cells of the innate, adaptive and regulatory immune systems, without adverse toxicity at therapeutic doses, is of significant importance in the field of cancer immunotherapy—specifically for developing anticancer vaccines. Previously, we demonstrated that 8Qm upon conjugation to polyacrylates can self-assemble to form microparticles. The microparticles upon single immunisation can trigger eradication of young tumour (3 days) from mice [4, 7, 8]. However, when vaccination was delayed to 7 days post tumour implantation, mice survival rate dropped significantly even when boost immunisation was used [9].

Formulation of the 8Qm-polymer conjugate in liposomes, improved tumour eradication potency with 3 among 5 mice tumour free two months post tumour implantation [10]. These previously reported polyacrylate systems have faced serious limitations in regard to commercial application because they are often not biodegradable, typically have undefined stereochemistry and contain a variable number of units in each polymer. Although this is typical for classical polymers, batch-to-batch variability affects in vitro and in vivo results and makes them unsuitable for clinical trials.

While a pHAA system is still classified as polymeric, it is tremendously different than any other polymeric system used for vaccine delivery. The application of fully defined polymer-based or natural hydrophobic amino acid serving as monomers may overcome all disadvantages of classical polymer-based delivery systems. As natural protein transmembrane domains are often leucine rich, thus we hypothesise that using polyleucine in such delivery system may allow anchoring of the conjugates to the liposomal membrane.

In this example, a new pHAA was attached to 8Qm peptide and anchored into a secondary delivery system, liposomes (see FIG. 14). A mannose targeting moiety was also incorporated into the liposomes, along with the pHAA-8Qm conjugate, to determine if vaccine candidates were more efficiently taken up by immune cells when compared to liposomes containing pHAA peptides alone. Indeed, mannosylated liposomes were efficiently uptaken by antigen presenting cells, induced maturation and completely eradicating 7-day-old tumour cells against model TC-1 tumour.

Materials and Methods

Materials

All chemical materials used in this study were analytical grade unless otherwise stated. Protected Fmoc amino acids and [(1-Bis(dimethylamino) methylene]-1H-1,2,3-triazolo[4,5-b]pyridinium 3-oxid hexafluorophosphate (HATU), were purchased from Mimotopes (Melbourne, Australia). Dichloromethane (DCM), diethyl ether, piperidine and trifluoroacetic acid (TFA) N,N′-dimethylformamide (DMF), N,N′-diisopropylethylamine (DIPEA), piperidine, HPLC grade acetonitrile and methanol were purchased from Merck (Darmstadt, Germany). Triisopropylsilane (TIS), acetic anhydride, low molecular weight chitosan (50-190 KDa), sodium alginate (low viscosity), erythrocytes lysing buffer, cholera toxin B subunit (CTB), pilocarpine, phosphate buffered saline (PBS), phenylmethyl-sulfonylfluoride (PMSF), antimouse IgG and IgA conjugated to horse-radish peroxidase were purchased from Sigma-Aldrich (St Louis, USA). IC Fixation buffer and Phenol-free IMDM Glutamax medium was obtained from Gibco® Life Technologies (CA, USA). Bovine serum albumin, anti-mouse CD16/CD32, PE/CY7-CD11C, BV605-F4/80, FITC-CD40, PE-CD80 and APC-CD86 were obtained from eBioscience (CA, USA). Dipalmitoylphosphatidylcholine (DPPC), cholesterol and dimethyldioctadecylammonium bromide (DDAB), Avanti mini extruder, PC membranes and filter supports were bought from Avantis polar, Inc. (Auspep Pty. Ltd, VIC, Australia). All other reagents were purchased from Sigma-Aldrich (Castle Hill, NSW, Australia). Cu wires were purchased from Aldrich (Steinheim, Germany). All other reagents were obtained at the highest available purity from Sigma-Aldrich (Castle Hill, NSW, Australia). Leu10-J8 peptide was synthesized as described elsewhere in this Example. ESI-MS was performed using a Perkin-Elmer-Sciex API3000 instrument with Analyst 1.4 software (Applied Biosystems/MDS Sciex, Toronto, Canada). Analytical RP-HPLC was performed using Shimadzu (Kyoto, Japan) instrumentation (DGU-20A5, LC-20AB, SIL-20ACHT, SPD-M10AVP) with a 1 mL min⁻¹ flow rate and detection at 214 nm and/or evaporative light scattering detector (ELSD). Separation was achieved using a 0-100% linear gradient of solvent B over 40 min with 0.1% TFA/H₂O as solvent A and 90% MeCN/0.1% TFA/H₂O as solvent B on either a Vydac analytical C4 column (214TP54; 5 μm, 4.6 mm×250 mm) or a Vydac analytical C18 column (218TP54; 5 μm, 4.6 mm×250 mm). Preparative RP-HPLC was performed on Shimadzu (Kyoto, Japan) instrumentation (either LC-20AT, SIL-10A, CBM-20A, SPD-20AV, FRC-10A or LC-20AP×2, CBM-20A, SPD-20A, FRC-10A) in linear gradient mode using a 5-20 mL/min flow rate, with detection at 230 nm. Separations were performed with solvent A and solvent B on a Vydac preparative C18 column (218TP1022; 10 μm, 22 mm×250 mm), Vydac semi-preparative C18 column (218TP510; 5 μm, 10 mm×250 mm) or Vydac semi-preparative C4 column (214TP510; 5 μm, 10 mm×250 mm). The particle size distribution and measurement of the average particle size were analyzed using a dynamic light scattering (DLS) (Malvern Instruments, England, UK). Multiplicate measurements were performed, and the average particle size was represented.

### Synthesis of Peptides

D-8Qm and 8Qm were synthesized as previously reported [10].

### Synthesis of Leu-8Qm

Leu-8Qm was synthesised by manual stepwise SPPS on rink amide MBHA resin (substitution ratio: 0.52 mmol/g, 0.1 mmol scale, 0.192 g) using HATU/DIPEA Fmoc-chemistry. The product was purified with RP-HPLC C-4 column, with a 50-80% solvent B gradient over 30 minutes. t_{R}=32 min, purity >95%. Yield: 32%. ESI-MS: m/z 1417.7 (calculated 1417.24) [M+2H]²⁺; 945.7 (calculated 945.16) [M+3H]3+; MW=2832.48.

### Synthesis of DOPE-PEG3.4K-alkyne (4)

A mixture of DOPE (1, 11.4 mg, 456 μL, 25 mg/mL stock in chloroform, 15 μmol, 1 equiv.) and triethylamine (11.6 mg, 16 μL, 114 μmol, 7.6 equiv.) was added dropwise to a solution of NPC-PEG3.4K-NPC (2, 250 mg, 73.5 μmol, 4.9 equiv.) in anhydrous chloroform (3 mL). The mixture was stirred for 14 h at room temperature under nitrogen atmosphere. A mixture of propargylamine (47 μL, 40.4 mg, 735 μmol, 49 equiv.) and triethylamine (307 μL, 223 mg, 2.205 mmol, 147 equiv.) were then added to the reaction mixture and stirred for another 14 h at room temperature under a nitrogen atmosphere. The reaction mixture was evaporated in vacuo and the residue was flushed with nitrogen gas and dried on freeze dryer overnight. The residue was taken up with water (1 mL), vortexed, and then purified by size exclusion sepharose column chromatography CL-4B (50 cm column, 2 mL/fraction, eluted with degassed MilliQ water and the fractions were detected by TLC 30% methanol in chloroform and detected by Dragendorff's stain). After freeze drying, the product DOPE-PEG3.4K-alkyne (4) was obtained as a brown solid (56 mg, 87%). ¹H NMR (500 MHz, CDCl₃) δ 6.70 (s, 1H), 5.37 (s, 1H), 5.35-5.26 (m, 3H), 5.18 (s, 1H), 4.39-4.30 (m, 1H), 4.26-4.16 (m, 2H), 4.16-4.10 (m, 1H), 3.96-3.91 (m, 2H), 3.77-3.73 (m, 1H), 3.73-3.53 (m, 152H), 3.50-3.44 (m, 1H), 3.34 (s, 1H), 2.31-2.23 (m, 6H), 2.22 (t, J=2.5 Hz, 1H), 1.97 (q, J=6.5 Hz, 6H), 1.61-1.51 (m, 3H), 1.33-1.18 (m, 40H), 0.85 (t, J=7.0 Hz, 6H). ¹³C NMR (125 MHz, CDCl₃) δ 173.4, 130.0, 129.7, 79.8, 71.4, 70.8, 70.5, 69.9, 69.4, 64.3, 34.2, 34.0, 31.9, 30.7, 29.72, 29.70, 29.5, 29.3, 29.24, 29.21, 29.13, 29.10, 29.08, 27.18, 27.16, 24.87, 24.82, 22.6, 14.1. The product was detected by MALDI-TOF mass spectrometry (m/z=4100).

### Synthesis of 1-Azido-3,6-dioxaoct-8-yl 2,3,4,6-tetra-O-acetyl α-D-mannopyranoside (8)

This molecule was synthesized as per protocol [11]. In a dry round bottom flask (25 mL), boron trifluoride diethyl etherate (621 μL, 714 mg, 5.03 mmol, 3 equiv.) was added dropwise to a solution of mannose pentaacetate (5, 654 mg, 1.68 mmol, 1 equiv.), and 2-[2-(2-chloroethoxy)ethoxy]ethanol (6, 487 μL, 565 mg, 3.35 mmol, 2 equiv.) in anhydrous dichloromethane (10 mL) at 0° C. under nitrogen atmosphere. The reaction mixture was heated to 50° C. and stirred for 22 h. The reaction mixture was taken up with DCM (100 mL) and washed with saturated NaHCO₃ (100 mL×2), brine (100 mL×2), dried over anhydrous Mg₂SO₄ and then concentrated to give brown crude oil of chloro derivative 7. TLC R_{f}: 0.43 (1:1 Hexane/EtOAc, stained with molybdenum blue). The compound was purified by automatic flash column chromatography (12 G silica; 36 mL/min; 0-50% EtOAc/hexane over 50 min). The product 7 was obtained as a viscous light yellow oil, 540 mg, 65%.

The isolated product 7 (425 mg, 0.85 mmol, 1 equiv.) was then dissolved in anhydrous DMF (20 mL) into which a mixture of sodium azide (267 mg, 4.25 mmol, 5 equiv.) and tetrabutylammonium iodide (TBAI) (314 mg, 0.85 mmol, 1 equiv.) was added and then stirred at 87° C. for 17 h under nitrogen atmosphere. The solvent was removed and the residue was purified by automatic flash column chromatography (12 G silica; 36 mL/min; 0-50% EtOAc/hexane over 50 min) to give the azide Compound 8 (357 mg, 83%) as a colourless oil; R_{f}: 0.41 (1:1 EtOAc/hexane). The ¹H NMR (CDCl₃, 500 MHz) is in agreement with that reported by Guo [11].

### Synthesis of 1-Azido-3,6-dioxaoct-8-yl α-D-mannopyranoside (9)

Sodium metal (catalytic amount) was added to a solution of the acetylated mannose-azide 8 (357 mg, 0.71 mmol) in anhydrous MeOH (15 mL). After stirring for 3 h at RT under nitrogen atmosphere, the reaction mixture was diluted with MeOH and neutralised with Amberlite® IR-120H ion-exchange resin, filtered and then concentrated in and freeze dried to produce the product 9 (170 mg, 71%) as a colourless oil. The ¹H NMR (CDCl₃, 500 MHz) is in agreement with that reported by Guo [11].

### Synthesis of DOPE-PEG3.4K-mannose (10)

A mixture of Mannose-Azido-Triethylene Glycol 9 (8.3 mg, 25 μmol) and the DOPE-PEG3.4K-alkyne 4 (5 mg, 1.2 μmol, 1 equiv.) was dissolved in DMF (1 mL), and copper wire (42 mg) was added. The air in the reaction mixture was removed by nitrogen bubbling for 30 sec. The reaction mixture was covered and protected from light with aluminium foil and stirred for 3 h at 50° C. under nitrogen atmosphere. TLC R_{f}: 0.2 (20% MeOH and CHCl₃, visualized by UV and stained with molybdenum blue). The wires were filtered off from the warm solution and washed with 1 mL of DMF. The DMF solution was slowly added to 4 mL water (0.005 mL/min). Micelles formed through the self-assembly process and dialyzed against water (1 L) a 2 KDa dialysis bag for 14 h. After lyophilization, the pure DOPE-PEG3.4K-mannose (10) was obtained as an amorphous white powder (2 mg, 37%). ¹H NMR (CDCl₃, 500 MHz) δ 7.77 (1H, d, J 10 Hz), 6.94-6.82 (2H, m), 5.32 (2H, brS), 5.18-5.14 (1H, m), 4.87-4.71 (3H, m), 4.51-4.35 (3H, m), 4.25-4.20 (2H, m), 4.14-4.09 (1H, m), 4.05 (1H, s), 3.95-3.92 (2H, m), 3.86-3.84 (2H, m), 3.80-3.50 (94H, brS), 3.49-3.46 (1H, m), 3.36-3.30 (1H, m), 2.50-2.45 (4H, m), 2.32-2.24 (8H, m), 2.02-1.98 (4H, m), 1.26-1.23 (28H, d), 0.86-0.84 (6H, t, J 7.5 Hz). MALDI-TOF mass spectrometry (m/z=˜4550).

Formulation of Antigen Loaded Nano-Liposomes

Liposomes were formulated using a lipid hydration method followed by extrusion or sonication to form uniform sized particles. Dipalmitoylphosphatidylcholine (DPPC), didodecyldimethylammonium bromide (DDAB), and cholesterol was dissolved in chloroform to final concentrations at 10 mg/mL, 5 mg/mL and 5 mg/mL, at a weight ratio of 5:2:1 respectively. All components were mixed in a round bottom flask along with 1 mg/1 mL of D-8Qm or Leu-8Qm or DOPE-PEG3.4K-mannose and lyophilized antigen dissolved in methanol. The organic solvent was slowly evaporated using rotary evaporator and lyophilised overnight to complete solvent removal. Liposome films were rehydrated with 900 μL of Millipore endotoxin-free water. L1 was then sonicated 4 times with a micro sonicate probe (40% of power, 20 s of pulser for 2 mins) to obtain homogenous liposomes. L2 and L2M were extruded through a 200 nm pore size polycarbonate membrane. Leu-8Qm peptide was also self-assembled into nanoparticles. Prior to injection, 100 μL of 10×PBS was added to the formulation producing 1 mg/mL concentration.

Characterisation of Nanoliposomes

The particle size, zeta potential and polydispersity of vaccine candidates were measured by dynamic light scattering (DLS) using zetasizer (Nano ZX, Marvern, England). The particle morphology of vaccine candidates was examined using transmission electron microscope (TEM) (HT7700 Exalens, HITACHI Ltd., Japan) after vacuum-drying. Briefly, the sample were diluted in pure distilled water (1:100) and dropped directly on a glow-discharged carbon coated copper grid and then stained with 2% uranyl acetate. The samples were observed with magnification of 200.0 k×.

Mice and Cell Lines TC-1

TC-1 cells (murine C57BL/6 lung epithelial cells transformed with HPV-16 E6/E7 and ras oncogenes) were obtained from TC Wu. TC-1 cells were cultured and maintained at 37° C./5% CO2 in RPMI 1640 medium (Gibco) supplemented with 10% heat inactivated fetal bovine serum (Gibco). Female C57BL/6 (6-8 weeks old) mice were used in this study and purchased from Animal Resources Centre (Perth, Western Australia). The animal experiments were approved by the University of Queensland Animal Ethics committee (UQDI/TRI/351/15) in accordance with National Health and Medical research Council (NHMRC) of Australia guidelines.

In Vivo Tumour Treatment Experiments

C57BL/6 mice (8 per group) were challenged subcutaneously in the flank with 2×10⁵/mouse of TC-1 tumour cells, 1×10⁵/mouse of TC-1 tumour cells each side. After 7 days, mice were injected in the flank with vaccine candidates. Each mouse received 100 μg of peptide or liposome formulation containing 100 μg of antigen. L2M vaccinated mice received 100 μg of Leu-8Qm antigen with 2 μg of DOPE-PEG3.4K-mannose targeting ligand. L1 was used as a positive control. A negative control group was administered 100 μL of PBS. The mice received single dose of vaccine. The size of the tumour was measured by palpation and callipers every two days and reported as the average tumour size across the group of five mice or as tumour size in individual mice. Tumour volume was calculated using the formula V (cm³)=3.14×[largest diameter×(perpendicular diameter)²]/6. The mice were euthanized when tumour reached 1 cm³ or started bleeding to avoid unnecessary suffering. Another new set of C57/BL6 mice (6 per group) were immunised using antigens as above.

IFN-Gamma ELISPOT Assays

ELISPOT plates were coated with 5 μg mL-1 IFN-g capture antibody (clone) in PBS at 4° C. overnight. The plates were then blocked with RPM/20% FCs at room temperature for 3 hours. Splenocytes from C57/BL/6 mice were harvested from the spleens of naïve and immunised mice. The red blood cells were depleted using red blood cell lysing buffer (0.155 M ammonium chloride in 0.01 M Tris-HCl buffer, Sigma). Splenocytes were then resuspended in RPMI (sigma) supplemented with 20% FACs (100 UmL-1 penicillin and 100 μg mL-1 streptomycin and 50 μM b-mercaptoethanol. The cells were plated at 5×10⁵ cells per well in triplicate on ELISPOT plates. E7 peptide (10 μg mL-1) was added alongside 10 ng mL-1 rhIL-2 to a final volume of 200 μL per well. Plates were incubated for 8 hours at 37 C, washed and biotinylated IFN-g detection antibody in 1% BSA added at room temperature, plates were washed again and bound cytokine was visualised with 3-amino-9ethylcarbozole. Spots were counted with an ELISPOT reader.

Statistical Analysis

All data were analyzed using GraphPad Prism 7 software. Kaplan-Meier survival curves for tumour treatment experiments were applied. Differences in survival treatments were determined using the log-rank (Mantel-Cox) test, with p<0.05 considered statistically significant. ELISPOT statistical analysis was performed using a one-way analysis of variance (ANOVA) followed by Tukey's multiple comparisons test (ns, p>0.05; *, p<0.05; **, p<0.01; ***, p<0.001, ****, p<0.0001).

Results

Synthesis and Characterisation

Leu10-8Qm peptide was synthesized using Fmoc SPPS chemistry to a high purity (<95%). Polyacrylate D8 was conjugated to 8Qm using copper-catalysed alkyne-azide cycloaddition (CuAAC) reaction and self-assembled into particles via the solvent replacement method as reported previously [4, 10]. Dialysis was performed in water for 3 days to remove residual peptide, copper and organic solvents. Elemental analysis was used to confirm formation of the product, as the conjugate contained a higher nitrogen/carbon ratio compared to the polyacrylate alone. The theoretical and observed (N/C) ratio were used to calculate the exact substitution of the polymer core with the peptide epitopes. The observed nitrogen to carbon ratio for D-8Qm (N/C=0.07) which is higher than the polymer alone (N/C=0.017). DOPE-PEG3.4K-mannose was successfully synthesized. In a one-pot reaction, the alkyne derivative 4 was synthesized in two steps (Scheme 1). In the presence of triethylamine (TEA), a large excess of nitrophenylcarbonyl-PEG3.4K-nitrophenylcarbonyl (NPC-PEG3.4K-NPC, 2) has been treated with DOPE (1) to produce mono NPC derivative 3. By treating the reaction mixture with an excess of a mixture of propargyl amine and TEA, all the NPC groups were substituted by propargyl moieties to provide Compound 4 along with propargyl-PEG3.4K-propargyl as a side product. The reaction was monitored by TLC using 30% methanol in chloroform with the products detected by Dragendorff's stain. Product 4 was separated from the excess dipropargyl derivative of 2 by size exclusion sepharose column chromatography (CL-4B) due to the ability of 4, rather than the dipropargyl derivative of 2, to form micelles. The pure product 4 was collected as a brown solid in an excellent yield (87%) and characterized by NMR and MALDI-TOF mass spectrometry (m/z=˜4100).

Mannose-Azido-Triethylene Glycol 9 was synthesized through three steps following Guo et al., (Scheme 1). Briefly, acetylated mannose 5 was treated with 2-[2-(2-chloroethoxy) ethoxy]ethanol 6 in presence of boron trifluoride diethyl etherate to produce chloro derivative 7, 65% yield. Heating 7 with sodium azide provided the azido derivative 8, 83% yield. The removal of acetyl groups was performed by using sodium metal in methanol followed by neutralization using Amberlite® IR-120H ion-exchange to afford product 9, 71% yield.

Conjugation of 9 with DOPE-PEG3.4K-alkyne (4) afforded DOPE-PEG3.4K-mannose (10) (Scheme 1), which was used as a mannose receptor targeting ligand in our delivery system to improve the targeting and uptake of vaccine delivery efficiency. The product 10 (m/z=4500) was characterized by MALDI-TOF mass spectrometry (m/z=˜4500).

D-8Qm, Leu-8Qm and DOPE-PEG3.4K-mannose were incorporated into the liposomal bilayer during thin film lipid formulation. Lipid films were hydrated and sonicated (L1) as previously reported or extruded (L2, L2M) with 200 nm pore membrane to form uniform-sized nanoparticles. Leu-8Qm was also self-assembled in the presence of water due to the hydrophilic peptide and pHAA properties to produce particles without liposome contents for comparison.

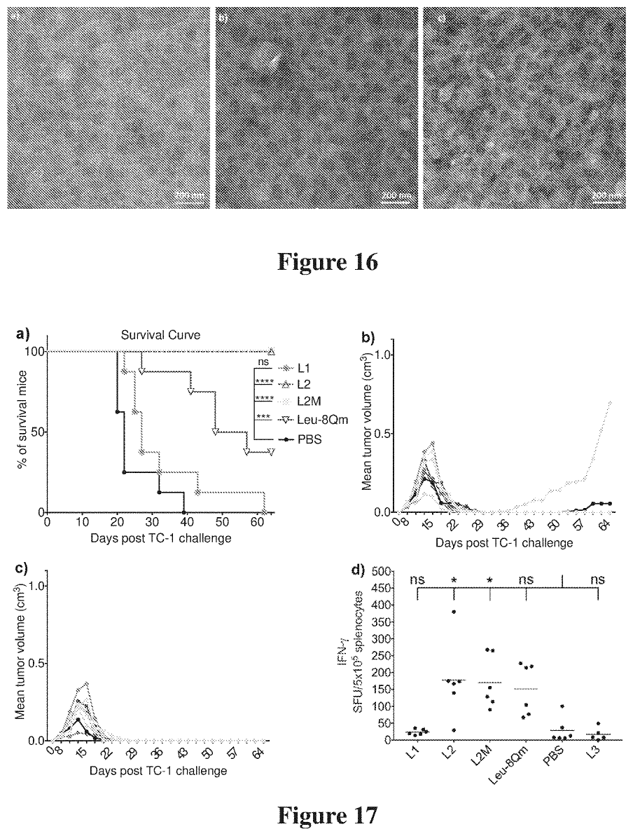

The size, surface charge and morphology of the candidates were analyzed using dynamic light scattering (DLS) (Table 1a) and TEM (FIG. 16). L1 formed particles of a size 120 nm with low PDI (0.15) and a charge of +60 mV after sonication. L2 and L2M formed particles of similar size 140 nm and 200 nm, with a PDI of 0.3 and 0.23 respectively. Both had similar surface charge of +47 mV. The Leu-8Qm peptide alone, formed large particles or aggregates with a high PDI 0.6 and a negative charge of −10 mV. TEM demonstrated typical liposome spherical structures, uniform in size in the solution, similar as observed by DLS.

TABLE 1a
Physicochemical characterisation of vaccine candidates.

| Compound | Size | PDI | Charge |
|---|---|---|---|
| Blank liposomes | 165 ± 4 | 0.06 ± 0.01 | 60.6 ± 2 |
| Leu-8Qm | 4000 ± 150 | 0.6 ± 0.1 | −10 ± 5 |
| L1 | 120 ± 2 | 0.15 ± 0.02 | 56.6 ± 5 |
| L2 | 140 ± 3 | 0.23 ± 0.01 | 47 ± 1 |
| L2M | 200 ± 8 | 0.3 ± 0.04 | 47 ± 1 |

In Vivo

The therapeutic effect of the conjugates on established HPV tumour was evaluated in a mouse model of 8-week-old female C57/BL6 mice. Average tumour growth was fast, with only 25% survival rate of mice treated with PBS at day 21. The tumour growth rate was significantly lower in mice immunised with the pHAA delivery system containing 10 leucine amino acids and liposome components. Alone, Leu-8Qm, was not sufficient at destroying tumour cells with all mice exceeding 1 cm³ on day 57, however when anchored into liposomes L2, all mice survived for 64 days. The L2 also triggered shrinking of existing tumours, however, two mice started re-growing tumour cells after day 34 and 56. Moreover, L2M triggered significant shrinking of existing tumours, to no visual tumour growth. After 64 days, no mice regressed, showing no tumour cell regrowth for the full time period. Mice immunised with L2 and L2M produced IFN levels significantly higher than Leu-8Qm, positive L1 and negative control PBS (FIG. 17).

Discussion

As nanoparticles have been shown to have promising self-adjuvanting properties, amphiphiles that self-assemble into nanoparticles are often used in peptide vaccine delivery. Liposomes are usually formulated as nanoparticles, mimicking the properties of pathogens, and have the ability to induce cell-mediated immune responses. Liposomes consist of natural lipids which, when in the presence of water, can self-assemble into particles. During this self-assembly process, other hydrophobic moieties can embed themselves into the hydrophobic component of the nanoparticle, displaying hydrophilic peptides on the surface of the liposome. Here, we examine the ability of a new fully natural, peptide delivery system to treat 7-day old cancer in mice.

We designed and synthesized fully natural, peptide-based conjugates and encapsulated them into liposomes. The 8Qm epitope derived from HPV-16 E7 protein contains a CTL epitope (CD8+ CTL) and a T helper (CD4+) epitope in the single sequence. This peptide has shown promise as an antigen at stimulating immune responses against tumour cells. D-8Qm has shown limited success at tumour eradication in mice [4, 7, 8]. Incorporation of D-8Qm into liposomes L1 was an efficient self-adjuvanting delivery system as an anticancer vaccine for treatment of 7-day old tumours [10]. Previously, L1 significantly increased mice survival from 0% compared to 80% mice survival with 60% of mice tumour free 60 days post tumour implantation. Commonly used potent adjuvant Incomplete Freud's Adjuvant mixed with 8Qm had significantly less survival than L1 thus, was used as the positive control in this study. However, in the current study, L1 formed nanoparticles of similar size (120 nm) and PDI (0.15), however much higher charger (+56.6 mV) than previously reported [10]. The substitution rate of the polymer core to peptide epitope was calculated showing only 4 out of the 8 arms on the polyacrylate were substituted with 8Qm peptide. It is suggested that the lower substitution rate of D-8Qm to the polymer core decreased the number of negatively charged 8Qm peptide in L1, thus increasing the surface charge compared to the previous study. Mice immunised with L1 displayed tumours exceeding 1 cm³ within 62 days. Thus, the higher charge and lower efficacy of L1 may be attributed to the decreased substitution rate of peptide to polymer. As the polymer units are variable between each synthesis, working with non-biodegradable polymers can be difficult for repetition of experimental data. Inconsistency in tumour growth between experiments and variable sensitivity of C57/BL6 mice to TC-1 tumour also may have had an influence on tumour growth.

Thus, development of a natural peptide-based delivery system may overcome all disadvantages of classical polymer-based delivery systems. Concurrent data shown by D-8Qm and L1, inclusion of peptide into liposomes was essential for significant tumour eradication. Alone, Leu-8Qm increased survival rate of tumour infected mice, however all mice succumbed to the cancer by day 57. The anchoring of Leu-8Qm into liposomes (L2) shrunk tumours and prolonged mice survival. While all mice survived 64 days, 25% of the mice started to regrow tumour cells after day 34 and 56. Incorporation of mannose targeting moiety (L2M) improved the performance of the L2, triggering significant shrinking of existing tumours, to no visual tumour growth.

Thus, the liposomal formulation of the pHAA-system along with a mannose targeting moiety was important in improving the therapeutic properties of the vaccine candidate against HPV-related cancer. Further, we examined the recall IFN-gamma production by CD8+ T cells from immunised mice in response to MHC class I restricted E7 peptide restimulation by ELISPOT. Leu-8Qm immunised mice significantly produced IFN levels, regardless of with peptide alone, liposome delivery system or additional mannose targeting receptor.

Herein, it was demonstrated that a fully defined, natural, amino acid-based peptide epitope, anchored into liposomes can be used to decrease tumour growth in mouse model. A formulation incorporating a targeting mannose receptor was able to eradicate cancer from mice when administered 7 days after tumour inoculation. Importantly, this multi-component peptide-based vaccine candidate against HPV was able to effectively destroy tumour cells in a single dose immunisation without the use of an additional adjuvant.

### Example 3

Peptide-based vaccines have the potential to overcome the limitations of classical whole pathogen-based vaccines such as allergic and autoimmune responses and difficulty in the production of essential biological materials [1]. Peptide vaccines consist of short, defined, synthetic epitopes derived from a specific target pathogen which are able to induce an immune response. They can be easily synthesised and purified in large scale. Alone however, these small peptides are not stable in vivo and poorly immunogenic, lacking the danger signals required for recognition by the immune system. Thus, small peptides need to be incorporated into a protective delivery/adjuvant system such as liposomes or polymers [2-4].

Currently, most commercially available vaccines are administered through invasive and inconvenient techniques, which may result in low patient compliance, especially in remote areas where neglected tropical diseases prevail. Oral delivery has been shown to have many advantages over other more invasive delivery methods, such as ease of administration, simplified production and storage, and improved efficacy for gastrointestinal tract (GIT) pathogens [5, 6]. Oral vaccines however, are exposed to the GIT, which is composed of bacteria, enzymes, and low pH, all which can degrade vaccine peptides. Successful vaccines also need to cross the epithelial layer and be exposed to sensing immune cells. Finally, oral delivery of antigens usually requires multiple doses, which might induce oral tolerance and reduce compliance. Therefore, special delivery systems are required for the delivery of antigens orally, especially for labile peptides.

Necator americanus is the most prevalent of the human hookworms and infects more than 0.4 billion people. The intestinal parasite attaches to the mucosa of the small intestine via their teeth or cutting plates and feed on human mucosal tissue and blood. This can cause long-term pathological consequences, for example iron-deficiency anaemia leading to serious debilitating conditions such as impaired neurological and cognitive functioning in chronically infected infants. Globally, hookworm infection is one of the most common tropical diseases, outranking dengue fever, schistosomiasis and leprosy in terms of disability adjusted life years (DALYs) [7]. Control of hookworm in endemic regions relies on mass administration to school-aged children of anthelminthic drugs such as albendazole. While chemotherapy is generally effective, reduced drug efficacy, continuous reinfection and the wide distribution of hookworms has prompted the need for alternative strategies to control the infection, such as vaccination [7]. Intriguingly, life-long exposure to hookworm infection does not stimulate robust protective immunity, and often the elderly have the heaviest worm burdens [8]. Promising anti-hookworm vaccine peptides have been reported based on Na-APR-1, the cathepsin D-like aspartic protease that is a responsible for degradation of human haemoglobin, which the worms use as nourishment. Problems with scale up production and protein aggregation [9] prompted the discovery of a synthesizable peptide derived from Na-APR-1, called A₂₉₁Y, which was the target of monoclonal antibodies that neutralize the proteolytic activity of the parent Na-APR-1 protein [10]. Recently, we demonstrated that a 22-mer peptide derived from A₂₉₁Y, called p3, when lipidated or mixed with adjuvant, stimulated production of Na-APR-1 proteolysis-neutralising antibodies upon subcutaneous immunisation [11, 12].

The use of fully defined, degradable natural polymers built from hydrophobic amino acids (HAA) can be utilised as a self-adjuvanting delivery system. Synthesis of a poly-HAA (pHAA) uses the classical solid phase peptide synthesis (SPPS) method, which allows for incorporation of peptide epitopes into the one molecule in a single procedure. A key advantage to this system is improved biodegradability and biocompatibility, as well as the ability to adjust properties of the pHAA unit (solubility, conformational etc.) as required by changing the type and number of incorporated HAAs. While we have recently demonstrated that the pHAA system can self-assemble into chain-like nanoparticle aggregates and serve as an injectable vaccine against Group A Streptococcus, the ability of such a system to deliver antigens orally has not yet been investigated.

In contrast to natural amino acids, unnatural amino acids such as lipoamino acids (LAAs), have been widely used in the lipid core peptide (LCP) system to generate immune responses against a variety of pathogens [13-18], including hookworm [11, 12]. LCP consists of peptide epitopes, a branching moiety, and LAAs linked together into a single vaccine peptide. LCP as an amphiphile has the ability to self-assemble into nanoparticles [19]. We showed that LCP nanoparticles were effective in inducing production of high antibody titres against GAS infection [20,21]. Nanoparticles are particularly attractive in vaccine design [22-24] as they can be effectively taken up by antigen presenting cells, stimulate adaptive immunity and have improved oral stability [6, 25, 26].

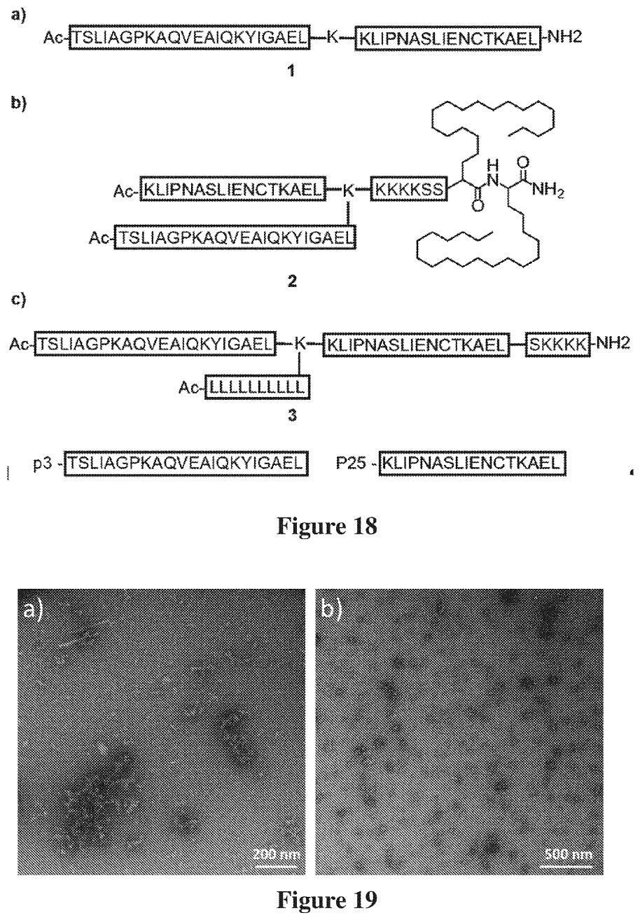

The aim of this Example was to create a single molecule-based self-adjuvanting oral vaccine against hookworm infection. We designed two vaccine candidates with peptide antigens conjugated to unnatural lipidic LAA and natural pHAA units, respectively. For this purpose, the p3 hookworm B cell epitope was coupled to a T helper peptide (P25; SEQ ID NO:8) with a lysine branching spacer (1) and attached to an LCP system containing 2-amino-d,l-eicosanoic acid [20] to produce lipopeptide 2 and a pHAA delivery system containing polyleucine to produce peptide 3 (FIG. 18).

A hydrophilic moiety built from lysine and serine was introduced to peptides 2 and 3 to increase the solubility and allow for self-assembly of peptides as both P25 and p3 are relatively hydrophobic. This moiety is identical to the traditional solubilizing unit used in the Pam3Cys adjuvant and its derivative peptides [27]. A similar approach has proven effective in the design of self-assembled LCP-based nanoparticles (15-20 nm) as a peptide-based vaccine against GAS [28]. Peptides 1-3 were synthesized using standard Boc-SPPS procedure [29].

Peptides 2 and 3 were self-assembled under aqueous conditions and their properties were measured using dynamic light scattering (DLS) and transmission electron microscopy (TEM) (FIG. 19). Peptide 2 formed particles of around 115 nm and 340 nm in size with a high polydispersity index of 0.4, similar to random aggregates as previously seen in LCP-based systems. Peptide 3 displayed a range of particle sizes (100-5000 nm) with a high PDI of 0.4, however, 100 nm nanoparticles were predominantly observed with TEM, (FIG. 19). The size of the particles can affect how they are absorbed through the GI tract and subsequently recognised and processed by antigen presenting cells, with particles of less than 500 nm being more efficiently taken up than larger particles [6, 30].

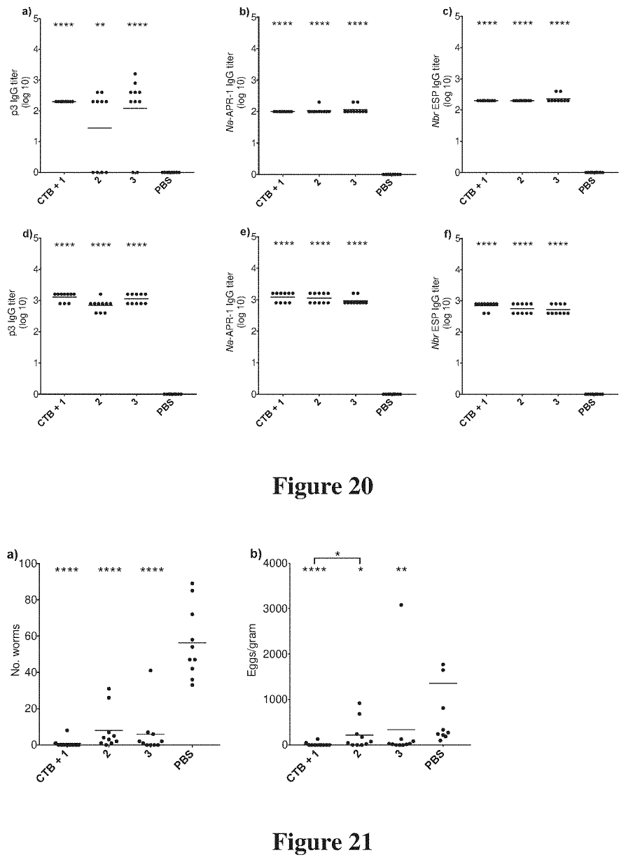

Next, to determine the antigenicity and vaccine efficacy of the conjugates, an animal model of human hookworm infection was employed. The human hookworm, N. americanus, does not naturally infect laboratory animals. N. brasiliensis on the other hand is a soil-transmitted nematode which is commonly called the “rodent hookworm”. It has a similar lifecycle and morphology to human hookworms, and its secretome is highly conserved with that of N. americanus [31]. Moreover, the p3-peptide of Na-APR-1 is completely conserved between the two parasites. The ability of the vaccine peptides to elicit a humoral response against hookworm was investigated. BALB/c mice (10 mice/group) were orally immunised with 100 μg per dose of peptides 2 or 3. The positive control group received 100 μg of peptide 1 formulated with 10 μg of cholera toxin subunit B (CTB), a potent mucosal adjuvant that is only approved for mouse studies due to its potential toxic side effects in other animal species. The negative control group received 100 μL of PBS. Following weekly oral dosing for a total of 6 weeks, immunised mice were subcutaneously challenged with 500 N. brasiliensis third-stage larvae (L3) (day 50) and left for 7 days. Blood and saliva samples were collected before challenge at day 48, 7 days after the final boost was administered, and post-challenge at day 56 (FIG. 20). A peptide-based vaccine not only needs to induce production of antibodies against the p3 peptide but those antibodies also need to recognise the parent N. americanus and N. brasiliensis proteins, Na-APR-1 and Nb-APR-1, respectively. Recombinant Na-APR-1 was available to us [32] whereas recombinant Nb-APR-1 has not been expressed but is present in its native form in N. brasiliensis excretory/secretory products (Nbr ESP) [31].

Similar IgG antibody titres against p3, Na-APR-1 and Nbr ESP were detected in mice treated with peptides 2, 3 and positive control CTB+1 respectively (FIG. 20). After challenge (day 56) all mice immunised with peptides 2 and 3 produced antibodies against the peptides and parent proteins, with similar titres to positive control mice treated with CTB+1 (FIG. 20). In comparison to the p3-specific vaccinated groups, the PBS negative control showed no pre-activation of the immune response, as demonstrated by no quantifiable induction of a natural immune response to the peptides in mice within 7 days after N. brasiliensis challenge infection. If a natural immune response develops, it can take up to two weeks, while the vaccinated groups produced higher antibody titers after just 7 days.

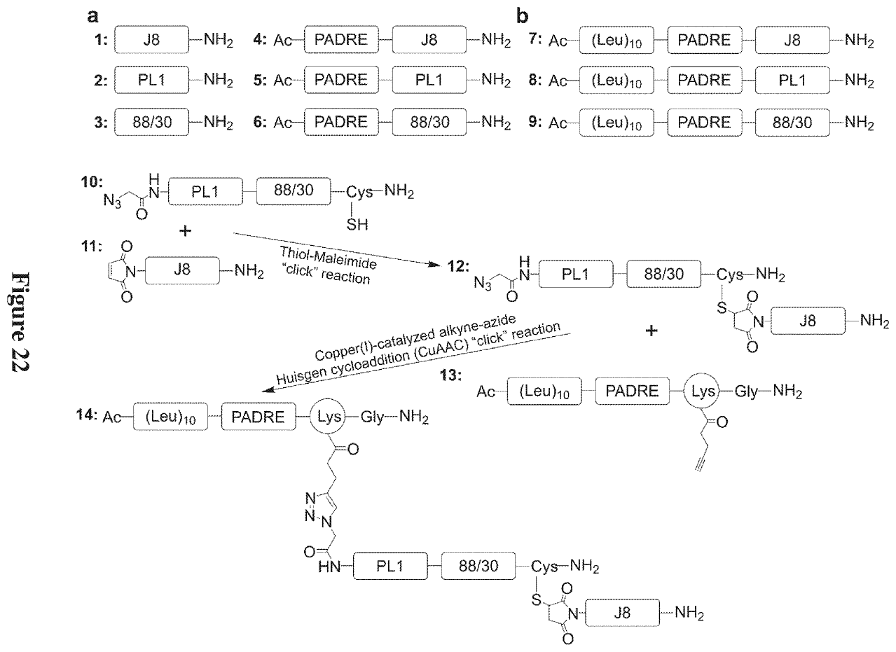

After infection (day 56), adult worms were collected from the small intestines and eggs were counted from faecal samples as described elsewhere.³³ The adult worm burdens were significantly reduced in the vaccinated groups 2 (85%), 3 (89%) and CTB+1 (98%) compared to the group that received PBS (FIG. 21). Faecal egg burdens were also significantly lower in all vaccinated groups 2 (84%), 3 (75%) and CTB+1 (98%) compared to the PBS negative control group. While there was no significant difference in worm burdens between mice that received the unnatural and natural amino acid-based delivery systems, peptides 2 vs 3, the pHAA-based vaccine candidate 3 has key advantages as it is fully biodegradable to natural metabolites, composed of readily available natural amino acids, and is a fully defined single molecule without racemic moieties.

Non-toxic and efficacious adjuvanting delivery systems for vaccines that can successfully deliver peptides orally and practically do not exist. Thus, the development of universal adjuvants is crucial to improve mucosal vaccination. Here, we have produced a single component, self-assembling, nanoparticle delivery systems that protects mice against a model hookworm infection. This single molecule-based delivery system was effective upon oral administration without the need of classically used a multicomponent delivery matrixes such as polymers [34] or liposomes [26]. Overall, the pHAA delivery system can be easily and cheaply produced and has shown promise in becoming a non-adjuvant, self-adjuvanting delivery system. According to our best knowledge, presented here pHAA-based delivery system is the first system based on peptides built from natural amino acids, which was effective for oral peptide delivery of vaccines.

### Experimental

Peptide 1

Peptide 1 was synthesised at 0.1 mmol on rink amide MBHA resin using Fmoc-SPPS as per protocol [35]. In summary, resin (substitution ratio: 0.79 mmol/g, 0.1 mmol scale, 0.127 g) was swelled overnight in N′N′-dimethylformamide (DMF). Synthesis was carried out using microwave assisted Fmoc-SPPS, SPS mode CEM Discovery reactor. The Fmoc group was removed using 20% piperidine/DMF for 2 and 5 minutes at 70° C. Amino acids were activated with 0.5 M 1-[bis(dimethylamino)methylene]-1H-1,2,3-triazolo[4,5-b] pyridinium 3-oxid hexafluorophosphate (HATU) (4 equiv, 0.8 mL) and N,N-diisopropylethylamine (DIPEA) (5.2 equiv, 91 μL) in DMF and added for 5 and 10 min coupling. Unreacted resin was acetylated with acetic anhydride, DIPEA and DMF (5:5:90) at 70° C., twice (5′ and 10′). The lipopeptide was dissolved in solvent B (acetonitrile/water (90:10)) prior to lyophilization. The product was purified by RP-HPLC using a C18 Vydac column with solvent gradient of 45-65% solvent B over 30 minutes. Analytical analysis was performed using a Shimadzu instrument (C4 column): t_{R}=min, purity >95%. Yield: 32%. ESI-MS: m/z 1423.6 (calc 1423.3) [M+3H]³⁺; 1067.8 (calc 1067.7) [M+4H]⁴⁺; 854.3 (calc 854.4) [M+5H]⁵⁺; 701.8 (calc 701.8) [M+6H]⁶⁺. MW=4267.1.

Peptide 2

Peptide 2 was synthesised at 0.2 mmol scale using the tert-butyloxycarbonyl (Boc) SPPS technique on MBHA resin in a similar manner as above. Resin (substitution ratio: 0.59 mmol/g, 0.2 mmol scale, 0.34 g) was swelled for two hours in DMF and DIPEA (5.2 equiv, 91 μL). LAA and amino acids were activated with HATU (4 equiv, 0.8 mL) and DIPEA (5.2 equiv, 91 μL) in DMF and added to resin for 5 and 10 min couplings. 4,4-dimethyl-2,6-dioxycyclohexylidene-2-amino-d,l-eicosanoic acid (Dde-C20-OH) was coupled to the resin using two 1-hour couplings at room temperature. The Dde protecting group was removed from the resin using hydrazine monohydrate (5%) for 10 min, 10 min and 20 min at room temperature. The remaining amino acids were coupled in the same manner. The Boc protecting group was removed using TFA twice, for 1 min each with stirring at room temperature. Boc-Lys(Fmoc)OH was introduced before synthesis of the P25 sequence allowing for peptide branching. The Fmoc group was removed using 20% piperidine upon completion of the P25 sequence, to allow for branching of the p3 peptide. Peptide was cleaved from resin using hydrofluoric acid (HF) (10 mLHF/g resin) at −8° C. in the presence of 5% (v/v) p-cresol and 5% (v/v) p-thiocresol. The lipopeptide was dissolved in acetonitrile/water (90:10) prior to lyophilization. The product was purified using RP-HPLC with a C4 column, with a 45-65% solvent B gradient over 30 minutes. t_{R}=41.1 min, purity >95%. Yield: 13%. ESI-MS: m/z 1886.3 (calc 1886.6) [M+3H]³⁺; 1415.6 (calc 1415.2) [M+4H]⁴⁺; 1132.5 (calc 1132.4) [M+5H]⁵⁺; 944.0 (calc 943.8) [M+6H]⁶⁺; 809.3 (calc 809.1) [M+7H]⁷⁺. MW=5657.0.

Peptide 3

Peptide 3 was synthesised at 0.2 mmol scale using the Boc SPPS technique on MBHA resin in the same manner as that described for peptide 2. The initial peptide using p3 and P25 sequences was first synthesised, followed by attachment of the pHAA chain. Boc-Lys(Fmoc)OH was introduced after synthesis of P25 allowing for peptide branching. Following synthesis of p3 to lysine-P25 sequence, the Fmoc side chain was removed using 20% piperidine and the poly-leucine moiety was attached using standard coupling procedure. The product was purified using RP-HPLC with a C4 column, with a 40-70% solvent B gradient over 30 minutes. t_{R}=30.2 min, purity >95%. Yield: 16%. ESI-MS: m/z 1522.0 (calc 1521.6) [M+4H]⁴⁺; 1217.6 (calc 1217.5) [M+5H]⁵⁺; 1014.9 (calc 1014.7) [M+6H]⁶⁺. MW=6082.5.

Characterisation

The size of the self-assembled particles was measured using photon correlation spectroscopy using a zetasizer 3000™ (Malvern Instruments, Malvern, UK). Morphology of vaccine peptides was evaluated using transmission electron microscope (TEM) (HT7700 Exalens, HITACHI Ltd., Japan) after vacuum-drying. Briefly, the samples were diluted in pure distilled water (1:100) and dropped directly on a glow-discharged carbon coated copper grid and then stained with 2% uranyl acetate. The samples were observed with magnification of 200 000.

Vaccination Scheme and Hookworm Challenge Model

Ten-week-old, male BALB/c mice (Animal Resources Centre, Perth, WA, Australia) were randomly divided into groups of 10 and orally immunised with freshly prepared vaccine formulations. Mice had free access to pelleted food and water. Peptides were given at a dose of 100 μg of 2 and 3 in 100 μL of water per mouse using a 20-gauge oral gavage tube on days 0, 7, 14, 21, 28 and 35. The negative control group received 100 μL PBS per mouse, while the positive control group received 100 μg control peptide 1 with 10 μg of CTB per mouse on the same dosing days. Two weeks after the final immunisation at day 49, mice were infected with 500 infective Nippostrongylus brasilisensis larvae (L3) in 200 μL of PBS subcutaneously in the scruff. N. brasiliensis was maintained in Sprague-Dawley rats (Animal Resources Centre, Perth, WA, Australia) as previously described [33, 36]. Infective L3 were freshly prepared from 2-week old rat faecal cultures. Seven days post-infection, mice were euthanised with C02 and blood (post-challenge), adult parasites from the small intestines and weighted faecal samples from the large intestines were collected.

Collection of Serum and Saliva

Blood samples were collected on days 48 and 56. Blood collection was via the tail artery into separation Z-Gel micro tubes, left for 1 hour at room temperature, and centrifuged for 5 minutes at 10,000 g. Final bleed was collected via cardiac puncture after mice were euthanized with C02 gas. The serum was removed and stored at −20° C. until analysis.

Evaluation of Antibody Responses

Enzyme-linked immunosorbent assay (ELISA) was performed to determine the antigen-specific IgG and IgA antibody titres in serum and saliva samples. All reactions were performed with 100 μL/well. Each well of a 96-well microtiter plate (Greiner Microlon® 600) was coated with 5 μg/mL of either p3 peptide, mature recombinant Na-APR-1 (provided courtesy by Pearson et al. [37]) or N. brasiliensis excretory/secretory proteins (Nbr ESP; as described by Eichenberger et al. [38]) diluted in 0.1 M sodium carbonate/bicarbonate (pH 9.6) for 2 hours at 37° C. Plates were washed three times with PBS/0.5% Tween 20 (PBST) and blocked with 5% skim milk to reduce non-specific binding overnight at 4° C. Samples at a 1:100 dilution for IgG in serum and at a 1:4 dilution for IgA in saliva were added for 1 hour at 37° C. All reactive samples were titrated to endpoint in two-fold serial dilutions. Plates were washed 3 times and horseradish peroxidase conjugated goat anti-mouse secondary antibody (anti-IgG at 1:4000 in PBST, Invitrogen #62-6 and anti-IgA at 1:2000 in PBST; Invitrogen #62-6720) was added for 1 hour at 37° C. Plates were washed 4 times and incubated with TMB substrate solution (Invitrogen 00-4201-56) at room temperature for 20 min. The antibody titres from the samples of the different groups were taken at the lowest dilution that exceeded an absorbance of 3 standard deviations of the mean absorbance from the negative control mice.

Statistical Analysis

Statistical analysis of antibody titres between groups was performed using a one-way analysis of variance (ANOVA) followed by Tukey's multiple comparisons test. Differences in adult worm and faecal egg burdens were analysed by non-parametric Mann-Whitney U test. GraphPad Prism 7.03 software (Graphpad Software Inc., CA, USA) was used for statistical analysis. Differences were significant if p<0.05.

### Example 4

Group A Streptococcus (GAS) bacteria are gram-positive pathogens known to colonize the throat or skin. GAS infections lead to a range of pyogenic diseases with varying degree of severity. The major target areas of these bacteria are the mucous membranes, skin, tonsil and the tissues around them. Commonly, GAS is known to cause pharyngitis, impetigo, pneumonia, meningitis, scarlet fever and cellulitis. However, this bacterium can also cause fatal invasive diseases such as Streptococcal Toxic Shock Syndrome (STSS), necrotizing (“flesh-eating”) fasciitis and septicaemia, and has potential to cause poststreptococcal (non-suppurative) sequelae that includes acute rheumatic fever, rheumatic heart disease (RHD), reactive arthritis and poststreptococcal glomerulonephritis. Due to the notorious nature of the infection, GAS was listed as the top 10 leading cause of infectious-related death in human, with global burden of 8 million patients annually (2013 data) and estimation of 319,400 deaths out of 33.4 million RHD cases alone (2015 and 2017 data).

To tackle this problem, vaccines against GAS have been extensively studied as vaccination is the best prophylaxis and a cost-effective medical intervention (e.g. ability to globally-eradicating smallpox). Traditional vaccine using whole organism (live, live-attenuated or inactivated pathogens) does has the advantage of long lasting immunity; however, can also induce autoimmunity or allergic responses as well as redundant biological materials that can reduce its efficacy as vaccines. In 1920s, a human trial was conducted using heat-killed GAS strains that was intravenously injected to adults and children. This attempt was a failure due to high reactogenicity by the presence of pyrogenic exotoxins of the crude vaccine design. Subunit vaccines have been introduced as a substitute to these traditional vaccines. They use only a part of the microorganism, such as carbohydrate, protein and peptide as vaccine antigen. Nevertheless, a protein-based subunit vaccine strategy using purified whole M protein (major virulence factor of GAS) or fragments of M protein still showed a presence of human tissue cross-reactivity despite its potency. Instead, recent GAS vaccine development uses peptide-based subunit vaccines, where a short sequence of amino acids derived from a selected antigen is used to trigger an immune response. Epitope J8 (28 amino acid peptide derived from the C repeat region of M protein) has been used as a vaccine antigen in many research studies, as well as in a human clinical study. Peptide-based vaccines are gaining popularity due to their multiple advantages, which include their safety profile, easy production of pure synthetic vaccine using chemical synthesis, and its stability in storage and for transport. However, the use of such minimal biological components has rendered peptides to be poor immunogens and require immunostimulants (adjuvants or delivery system) to overcome their immunogenicity.

In this Example, we design and formulate a multi-epitope vaccine against GAS that uses poly-hydrophobic amino acids (pHAAs) as a delivery system. This peptide-based self-adjuvating pHAAs has high efficacy and potency when conjugated to a GAS epitope [1], which contributed from the self-assembly of pHAAs-antigen conjugate into small nanoparticles and chain-like aggregated nanoparticle (CLAN). Nanoparticles are formed due to the amphiphilic properties of pHAA-antigen conjugates, formed when hydrophobic pHAAs conjugated to a hydrophilic epitope. This multi-epitope vaccine design incorporates the following components: different B-cell epitopes (J8, PL1 and 88/30) derived from GAS M protein, pan human leukocyte antigen-antigen D related (HLA-DR) binding epitope (PADRE) T helper cell epitopes to provides long-lasting memory immune response, and poly-leucine as a pHAAs moiety. The B-cell epitopes were selected from conserved (J8) and hypervariable (PL1 and 88/30) regions of M protein, which exclude the autoimmune sequence. J8 (QAEDKVKQSREAKKQVEKALKQLEDKVQ; SEQ ID NO:9) was derived from amino acids 344-355 of the M protein of M1 GAS strain, flanked with GCN4 DNA-binding protein sequences; whereas PL1 (EVLTRRQSQDPKYVTQRIS; SEQ ID NO:10) and 88/30 (DNGKAIYERARERALQELGP; SEQ ID NO:11) were derived from type-specific N-terminal from M protein of GAS strain M54 and 88/30 (strains that were first isolated from the Aboriginals of Northern Territory, Australia), respectively. Efficiency of the multi-epitope vaccine can be boosted by including universal synthetic PADRE T-helper epitopes (AKFVAAWTLKAAA; SEQ ID NO:7), which have high affinity to 15 out of 16 common HLA-DR types (a major histocompatibility complex (MHC) class II cell surface receptor). Poly-leucine was selected based on previous studies that showed leucine as the most effective pHAA system in comparison with other poly amino acids (valine and phenylalanine, alanine, glycine, glutamic acid, serine, proline and phenylalanine-leucine-alanine, as described herein) and commercial adjuvant (CFA, alum, AS04, MF59, as described herein). The physical mixture of individual J8, 88/30 and PL1 epitopes conjugated to PADRE and 10 copies of leucine, was compared to conjugated epitopes (J8, 88/30, PL1, PADRE) to poly-leucine (FIG. 22) on their ability to form nanoparticles and stimulate immune responses.

Material and Methods

Materials

Butyloxycarbonyl (Boc)-protected L-amino acids were purchased from Novabiochem, Merch Chemicals (Darmstadt, Germany) and Mimotopes (Melbourne, Australia). The 4-methylbenzhydrylamine (pMBHA) resin was obtained from Peptides International (Kentucky, USA). 1-[6-Bis(dimethylamino)methylene]-1H-1, 2, 3-triazolo[4,5-b]pyridinium-3-oxide hexafluorophosphate (HATU) was purchased from Mimotopes (Melbourne, Australia). Acetonitrile, dichloromethane (DCM), methanol, N,N-dimethylformamide (DMF), N,N-diisopropylethylamine (DIPEA), p-cresol, trifluoroacetic acid (TFA), were acquired from Merck (Hohenbrunn, Germany). Phosphate-buffered saline (PBS) was obtained from eBioscience (California, USA). C57BL/6 mice were purchased from the University of Queensland's Biological Resources (UQBR) (Queensland, Australia). Phenylmethylsulfonylfluoride (PMSF) were purchased from Thermo Scientific (Victoria, Australia). Goat anti-mouse IgG (H+L)-HRP (IgG HRP) conjugate was purchased from Millipore, Temacula (California, USA). Complete Freund's adjuvant (CFA) and goat anti-mouse IgA were purchased from Invivogen (San Diego, USA). Analytical-grade Tween 20 was purchased from VWR International (Queensland, Australia). Chloroform, o-phenylenediamine dihydrochloride (OPD) substrate, pilocarpine HCL, and all other reagents were purchased from Sigma-Aldrich (Victoria, Australia).

The chemical synthesis was carried out using microwave-assisted Boc solid phase peptide synthesis (SPPS) on SPS CEM Discovery reactor from CME Corporation (North Carolina, USA). The analysis of the peptides was done using electrospray ionization mass spectrometry (ESI-MS) on a LCMS-2020 Shimadzu (Kyoto, Japan) instrument (DGU-20A3, LC-20Ad×2, SIL-20AHT, STO-20A) or Analyst 1.4 software (Applied Biosystems/MDS Sciex, Toronto, Canada) (Perkin-Elmer-Sciex API3000), and analytical reverse-phase HPLC (RP-HPLC) on a Shimadzu (Kyoto, Japan) instrument (DGU-20A5, LC-20AB, SIL-20ACHT, SPD-M10AVP) using a Vydac analytical C18 (218TP54; 5 μm, 4.6×250 mm) or C4 (214TP54; 5 μm, 4.6×250 mm) column with compound detection at 214 nm. Purification was performed on a Shimadzu preparative RP-HPLC (Kyoto, Japan) instrument (LC-20AP×2, CBM-20A, SPD-20A, FRC-10A) with a 20.0 ml/min flow rate on a C18 (218TP1022; 10 μm, 22×250 mm) or C4 (214TP1022; 10 μm, 22×250 mm). Nanoparticle size and polydispersity were determined by dynamic light scattering (DLS) using Nanosizer Nano ZP instrument (Zetasizer Nano Series ZS, Malvern Instruments, United Kingdom) with disposable capillary cuvettes via Dispersion Technology Software (Malvern Instruments, United Kingdom). Further analysis of the nanoparticle size and morphology was conducted via particle-imaging captured using transmission electron microscopy (TEM; HT7700 Exalens, HITACHI Ltd., JEOL Ltd., Japan) by negative staining technique using 2% phosphotungstic acid and carbon-coated copper grids (Ted Pella, 200 mesh). Secondary structure was analysed in a 1 mm cuvette using Jasco J-710 Circular Dichroism Spectrometer. Enzyme-linked immunosorbent assay (ELISA) was conducted on a plate with plate reading absorbance measured using Spectramax microplate reader (Molecular Devices, USA). ELISA data were analysed using GraphPad Prism 7 software (GraphPad Software, Inc., USA). Multiple comparisons were performed using one-way ANOVA with Tukey's multiple comparison test. Data were considered significantly different at (*) p<0.05, (**) p<0.01, (***) p<0.001, (****) p<0.0001 among the studied group.

### Synthesis of Compounds 1-14

Compounds 1-11 and 13 (FIG. 22) were synthesised using microwave-assisted SPPS (70° C., 20 W) method via Boc chemistry on pMBHA resin.HCl (0.59 mmole/g substitution) at 0.1 mmol scale [2, 3]. Resin (0.017 g) was weighed and pre-swelled for 2 hours in DMF and DIPEA (6.2 equivalent). Coupling cycle includes the deprotection Boc group (2 minutes treatment with neat TFA, twice at RT), DMF wash, and amino acid (0.84 mmol/g, 4.2 equivalent) activation with 0.5M HATU (1.6 mL, 4 equivalent) and DIPEA (0.18 mL, 5.2 equivalent) and coupling to the resin using double coupling (5 minutes and 10 minutes). Special amino acid coupling conditions were applied for aspartic acid with 15 minutes double coupling at 50° C. These procedures were repeated until the desired peptides were achieved. Acetylation was performed after the addition of first and final amino acid by using 90% DMF, 5% DIPEA and 5% acetic anhydride. Formyl group from tryptophan amino acid was deprotected using 20% piperidine in DMF for 2 minutes and 5 minutes. The finished compounds were washed with DMF (3 times), DCM (3 times) and methanol, prior to drying in a desiccator overnight. After drying, the peptides were cleaved from the resin using anhydrous hydrogen fluoride (HF) and p-cresol as scavenger [4]. These compounds were precipitated and washed with cold diethyl ether. The precipitated compounds were then dissolved using solvent B (90% acetonitrile, 10% milli-Q water, 0.1% TFA) and then filtered. These compounds were purified using Shimadzu preparative RP-HPLC on C18 or C4 Vydac column, using solvent B gradients (specific gradients for each compound) for 25 minutes with compound detection at 214 nm. The purified compounds were then analysed using ESI-MS and analytical RP-HPLC on a C18 or C4 Vydac column, using 0-100% gradient of solvent B for 40 minutes with compound detection at 214 nm.

Additional reactions were performed to make Compounds 12 and 14. Pure Compound 10 (5.45 mg) was conjugated to pure Compound 11 (8.45 mg) using thiol-maleimide “click” reaction in a 1:1.2 molar ratio, to produce Compound 12. Both Compounds 10 and 11 were dissolved in 2.5 μL of DMSO, separately. Guanidine buffer (100 μL; 6M guanidine, 50 mM sodium phosphate buffer, 5 mM EDTA, 20% acetonitrile in water; pH 7.3) was added to each compound before mixing them together. The pH was adjusted to approximately 7.2-7.5 using 5% sodium hydroxide. Oxygen was removed by washing the flask thrice with nitrogen. The sample (30 μL) was collected every hour and analysed using analytical RP-HPLC with Vydac C4 column to monitor the progress of the reaction (reactant peak reduced and product peak grown). After the reaction finished, the mixture was purified using preparative RP-HPLC to isolate the pure Compound 12, which was analysed via ESI-MS and analytical RP-HPLC on C4 Vydac column.

Pure Compound 12 (6 mg) was conjugated to pure Compound 13 (3.2 mg) using copper(I)-catalyzed azide-alkyne Huisgen cycloaddition (CuAAC) “click” reaction [2] in a 1:1.7 molar ratio, to produce Compound 14. Both Compounds 12 and 13 were dissolved with 0.5 mL of DMF in a flask before adding 20 mg copper wire and magnetic stirring bar. The flask was covered using a septum stopper and oxygen was removed by washing the flask thrice with nitrogen. The flask was placed in a 50° C. oil bath and the mixture was stirred at 200 rpm. The sample (15 μL) was collected every hour once the mixture turned into the colour blue-green. The progress of the reaction, purification and analysis of Compound 14 were performed in the same manner as for Compound 12.

Compound 1 (pure) yield: 20%. Purity: 99%. Molecular weight: 3281.77. ESI-MS: [M+2H]²⁺ m/z 1641.5 (calculated: 1641.89), [M+3H]³⁺ m/z 1095.1 (calculated: 1094.92), [M+4H]⁴⁺ m/z 821.5 (calculated: 821.44), [M+5H]⁵⁺ m/z 657.5 (calculated: 657.35), Rₜ: 18.4 min (0-100% solvent B, 40 min, C18 column).

Compound 2 (pure) yield: 16%. Purity: 99%. Molecular weight: 2303.61. ESI-MS: [M+2H]²⁺ m/z 1152.6 (calculated: 1152.81), [M+3H]³⁺ m/z 768.5 (calculated: 768.87), [M+4H]⁴⁺ m/z 577.1 (calculated: 576.90). Rₜ: 17.7 min (0-100% solvent B, 40 min, C18 column).

Compound 3 (pure) yield: 16%. Purity: 99%. Molecular weight: 2285.56. ESI-MS: [M+2H]²⁺ m/z 1143.3 (calculated: 1143.78), [M+3H]³⁺ m/z 762.6 (calculated: 762.85), [M+4H]⁴⁺ m/z 572.4 (calculated: 572.39). Rₜ: 20.4 min (0-100% solvent B, 40 min, C18 column).

Compound 4 (pure) yield: 18%. Purity: 99%. Molecular weight: 4653.42. ESI-MS: [M+3H]³⁺ m/z 1552.4 (calculated: 1552.14), [M+4H]⁴⁺ m/z 1164.3 (calculated: 1164.36), [M+5H]⁵⁺ m/z 931.7 (calculated: 931.68), [M+6H]⁶⁺ m/z 776.6 (calculated: 776.57), [M+7H]⁷⁺ m/z 665.9 (calculated: 665.77). Rₜ: 25.0 min (0-100% solvent B, 40 min, C18 column).

Compound 5 (pure) yield: 16%. Purity: 99%. Molecular weight: 3675.26. ESI-MS: [M+2H]²⁺ m/z 1838.6 (calculated: 1838.63), [M+3H]³⁺ m/z 1226.0 (calculated: 1226.09), [M+4H]⁴⁺ m/z 919.9 (calculated: 919.82), [M+5H]⁵⁺ m/z 735.8 (calculated: 736.05). Rₜ: 20.2 min (0-100% solvent B, 40 min, C18 column).

Compound 6 (pure) yield: 15%. Purity: 97%. Molecular weight: 3657.21. ESI-MS: [M+3H]³⁺ m/z 1220.2 (calculated: 1220.07), [M+4H]⁴⁺ m/z 915.3 (calculated: 915.30). Rₜ: 18.3 min (0-100% solvent B, 40 min, C18 column).

Compound 7 (pure) yield: 15%. Purity: 98%. Molecular weight: 5785.02. ESI-MS: [M+4H]⁴⁺ m/z 1447.4 (calculated: 1447.26), [M+5H]⁵⁺ m/z 1158.2 (calculated: 1158.00), [M+6H]⁶⁺ m/z 965.0 (calculated: 965.17), [M+7H]⁷⁺ m/z 827.5 (calculated: 827.43). Rₜ: 30.9 min (0-100% solvent B, 40 min, C4 column).

Compound 8 (pure) yield: 22%. Purity: 99%. Molecular weight: 4806.86. ESI-MS: [M+3H]³⁺ m/z 1603.5 (calculated: 1603.29), [M+4H]⁴⁺ m/z 1203.0 (calculated: 1202.72), [M+5H]⁵⁺ m/z 962.2 (calculated: 962.37), [M+6H]⁶⁺ m/z 802.2 (calculated: 802.14). Rₜ: 37.9 min (0-100% solvent B, 40 min, C4 column).

Compound 9 (pure) yield: 15%. Purity: 98%. Molecular weight: 4788.81. ESI-MS: [M+3H]³⁺ m/z 1597.3 (calculated: 1597.27), [M+4H]⁴⁺ m/z 1198.2 (calculated: 1198.20), [M+5H]⁵⁺ m/z 958.9 (calculated: 958.76), [M+6H]⁶⁺ m/z 799.2 (calculated: 799.14). Rₜ: 30.5 min (0-100% solvent B, 40 min, C4 column).

Compound 10 (pure) yield: 15%. Purity: 96%. Molecular weight: 4758.33. ESI-MS: [M+4H]⁴⁺ m/z 1190.6 (calculated: 1190.58), [M+5H]⁵⁺ m/z 952.6 (calculated: 952.67), [M+6H]⁶⁺ m/z 794.3 (calculated: 794.06), [M+7H]⁷⁺ m/z 680.8 (calculated: 680.76), [M+8H]⁸⁺ m/z 596.0 (calculated: 595.79). Rₜ: 19.5 min (0-100% solvent B, 40 min, C18 column).

Compound 11 (pure) yield: 15%. Purity: 98%. Molecular weight: 3474.97. ESI-MS: [M+4H]⁴⁺ m/z 870 (calculated: 869.74), [M+5H]⁵⁺ m/z 696 (calculated: 695.99), [M+6H]⁶⁺ m/z 580 (calculated: 580.16), [M+7H]⁷⁺ m/z 498 (calculated: 497.42). Rₜ: 19.2 min (0-100% solvent B, 40 min, C18 column).

Compound 12 (pure) yield: 43%. Purity: 99%. Molecular weight: 8233.30. ESI-MS: [M+8H]⁸⁺ m/z 1031 (calculated: 1030.16), [M+9H]⁹⁺ m/z 916 (calculated: 915.81), [M+10H]¹⁰⁺ m/z 825 (calculated: 824.33), [M+11H]¹¹⁺ m/z 750 (calculated: 749.30), [M+12H]¹²⁺ m/z 687 (calculated: 687.11), [M+13H]¹³⁺ m/z 635 (calculated: 634.33). Rₜ: 18.4 min (0-100% solvent B, 40 min, C18 column).

Compound 13 (pure) yield: 20%. Purity: 98%. Molecular weight: 2785.59. ESI-MS: [M+3H]³⁺ m/z 930 (calculated: 929.53). Rₜ: 42.9 min (0-100% solvent B, 40 min, C4 column).

Compound 14 (pure) yield: 22%. Purity: 95%. Molecular weight: 11018.89. [M+10H]¹⁰⁺ m/z 1103 (calculated: 1102.89), [M+11H]¹¹⁺ m/z 1003 (calculated: 1002.72), [M+12H]¹²⁺ m/z 920 (calculated: 919.24), [M+13H]+¹³⁺ m/z 849 (calculated: 848.61), [M+14H]¹⁴⁺ m/z 788 (calculated: 788.06), [M+15H]^{15+ m/z} 736 (calculated: 735.59), [M+16H]¹⁶⁺ m/z 691 (calculated: 689.68). Rₜ: 26.5 min. (0-100% solvent B, 40 min, C4 column).

Secondary Structure Analysis

The secondary structure of the peptides in solution was analysed by CD spectrometry. Compounds 7-9 and 14 (0.1 mg/mL in PBS) were placed in quartz cuvettes. CD spectra measurement were taken with 1 mm path length at 23° C., and the data were collected between 198-260 nm [5]

Subcutaneous Immunisation in In Vivo Model

Six-week-old female C57BL/6 mice were purchased from the University of Queensland's Biological Resources (UQBR) (QLD, Australia). Mice were housed in the Australian Institute for Bioengineering and Nanotechnology (AIBN) Animal Facility. The mice were acclimatized for 7 days before any experiment was conducted. The mice were given 50 μL of Compounds 4-6 (mixture) and 14 via subcutaneous injection on the tail base on day 0, 21, 28 and 35. The amount of antigen for each compound varied based on the Table 2b. Positive control mice were given Compounds 1-3 dissolved mixture adjuvanted with CFA (1:1 volume ratio), whereas, negative control mice received 50 μL of PBS.

Collection of Serum

Serum samples were collected after immunisation on days 20, 27, 34, and 49 to measure antigen-specific IgG antibody titre. Blood was collected via tail tip (10 μL diluted in 90 μL of PBS on days 20, 27 and 34) and heart puncture (1 mL blood collected on day 49). The collected blood samples were centrifuged at 3,600 rpm for 10 minutes. Serum was collected from the supernatant and was kept at −80° C. until further analysis.

Antibody Titres Detection by ELISA

Antigen-specific antibodies IgG titres were detected using an (ELISA) assay as previously published. The plate was coated with either J8, PL1, or 88/30 (Compounds 1-3, respectively). Two-fold serial dilution was performed on antigen-coated plate, starting at 1:100 concentration of serum. Naïve mice sera were used as the control. Secondary antibody was added to the plate before adding OPD substrate. SpectraMax microplate reader was used to read the absorbance at 450 nm. IgG antibody titers were expressed as the lowest possible dilution with absorbance of more than three times the standard deviation, above the mean absorbance of control wells.

Results

Preparation and Characterization of Vaccine Candidates

Based on the adjuvating ability of 15-mer leucine to induce highly opsonic antigen-specific antibodies production in mice upon subcutaneous immunisation, herein new conjugates were designed. GAS B-cell epitope (either J8, PL1, or 88/30) conjugated to PADRE universal T-helper (Compounds 4-6, respectively) was designed as the antigens to induce antibody production. Linear Compounds 7-9 that were used as a mixture in the immunisation study, only bore 10 repeats of leucine to accommodate water-solubility issues with antigen bearing PL1 and 88/30 (Compounds 8 and 9) due to hydrophobicity of these peptides. Similarly, Compound 14 included 10 copies of leucine conjugated to PADRE, J8, PL1, and 88/30 antigens.

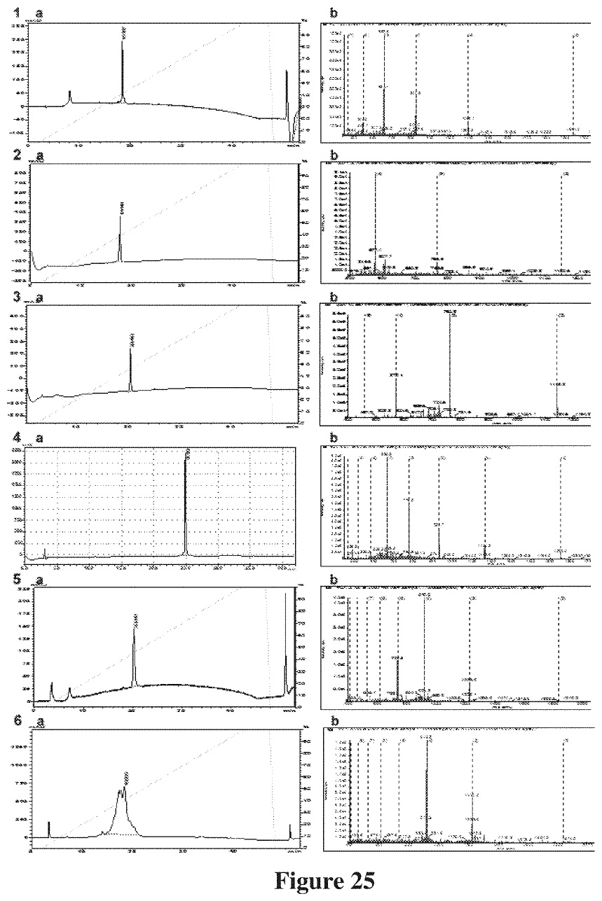

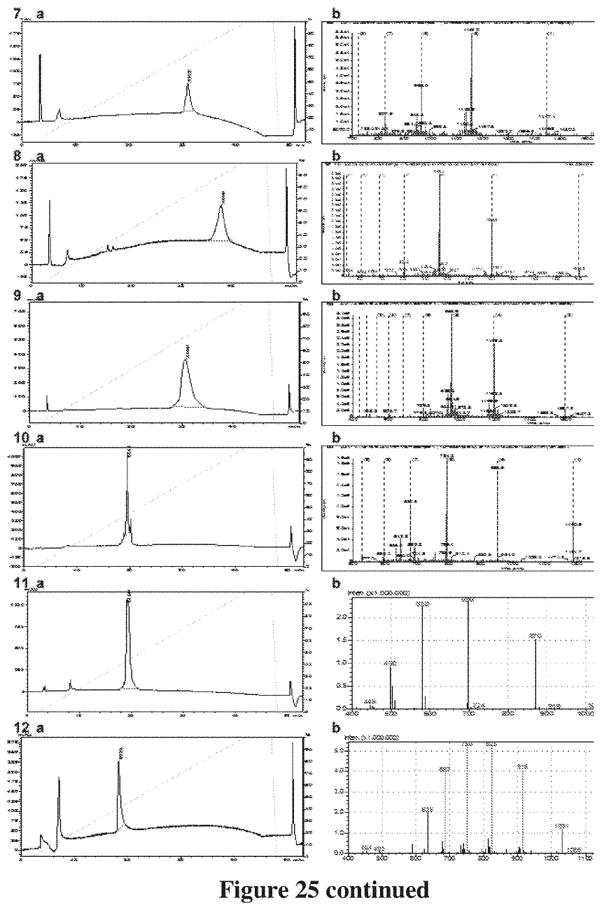

Compounds 1-11, and 13 were successfully synthesised using Boc-SPPS, and their structure and purity were confirmed using analytical RP-HPLC and ESI-MS. The observed mass on ESI-MS spectra were matched with the calculated mass of the desired compound. Additional reactions were performed to synthesise the multi-epitope Compound 14. Pure Compounds 10 and 11 were conjugated together using thiol-maleimide “click” reaction to produce Compound 12, which were purified and conjugated to Compound 13 using CuAAC “click” reaction to produce Compound 14. The progress of these click reactions was monitored using analytical RP-HPLC and ESI-MS. The reactants peaks on analytical RP-HPLC reduced while the product peak grew. Compounds 1-14 were purified using preparative RP-HPLC to achieve compound with purity greater than 95% (FIG. 25).

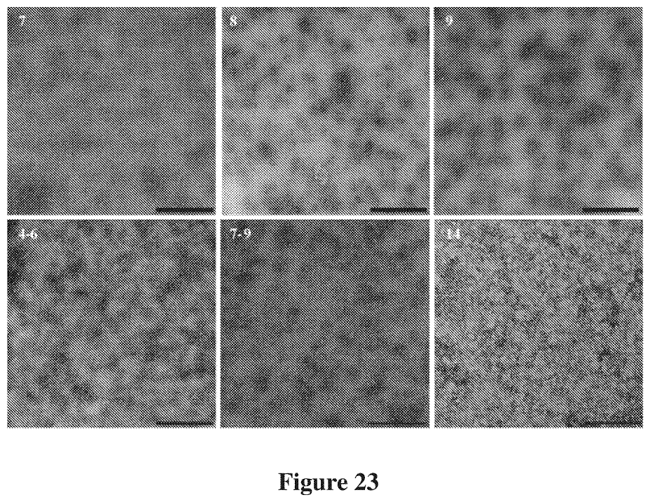

Mixture of Compounds 7-9 and individual Compound 14 were self-assembled to form nanoparticles in PBS. The compounds size, size distribution (PDI), and morphology were characterized using DLS and TEM (Table 1b, FIG. 23). In general, most of the compounds formed mixtures of small nanoparticles and chain-like aggregated nanoparticles (CLAN), similarly as has been shown in a previous study. All the compounds had a PDI>0.1.

TABLE 1b
Physicochemical characterization of the vaccine conjugates.

| | Particle size (nm) | | Polydispersity |
|---|---|---|---|
| Compounds | TEM | DLS | index (PDI) |
| Compound 4 | NA | 4 ± 0.1* | 0.52 ± 0.11 |
| | | 60 ± 13 | |
| | | 307 ± 50 | |
| Compound 5 | NA | 44 ± 8* | 0.52 ± 0.03 |
| | | 289 ± 25 | |
| | | 4678 ± 437 | |
| Compound 6 | NA | 177 ± 12* | 0.57 ± 0.19 |
| | | 1532 ± 46* | |
| Compounds 4-6 (mixture) | | 120 ± 113 | 0.36 ± 0.04 |
| | | 691 ± 110 | |
| Compound 7 | | 320 ± 10 | 0.50 ± 0.04 |
| Compound 8 | | 530 ± 10 | 0.31 ± 0.04 |
| Compound 9 | | 320 ± 42 | 0.58 ± 0.03 |
| Compounds 7-9 (mixture) | | 607 ± 9 | 0.13 ± 0.04 |
| Compound 14 | | 355 ± 16 | 0.26 ± 0.12 |

*= size detected when size distribution was measure by number.

Compound 7 self-assembled into small nanoparticles and CLAN (TEM image) with particle size (320 nm) from DLS. Similar particle size was measured by DLS for Compound 9. However, the TEM image for Compound 9 showed small nanoparticles with slightly larger and more visible aggregates compared to Compound 7, which has similar morphology to Compound 8 that has bigger particle size (530 nm). Mixture of Compounds 7-9 formed large particles of size 600 nm than multi-epitope conjugate Compound 14, with 350 nm aggregates measured by DLS.

Immunological Evaluation of the Conjugates

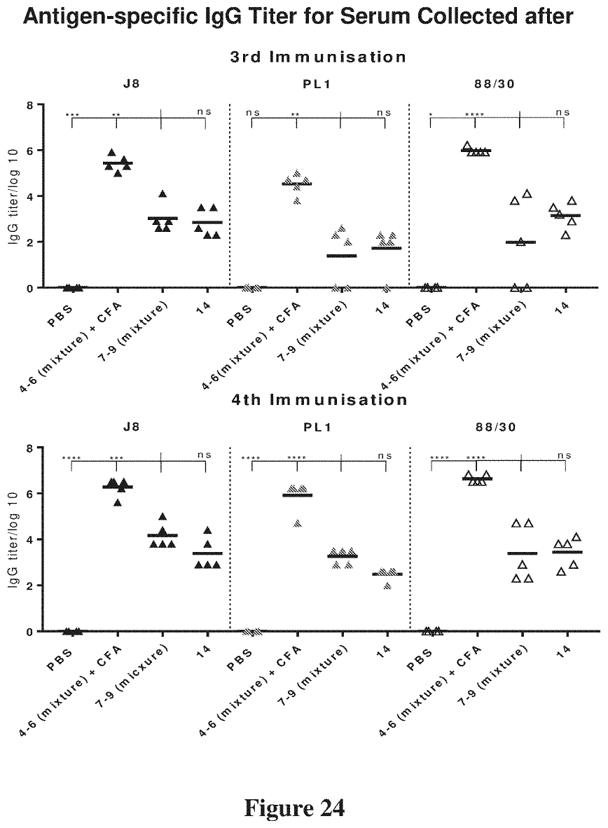

Immunological evaluation of polyHAA-antigen conjugates was performed on C571BL/6 using prime-boost vaccination strategy. Mice were subcutaneously immunised with Compounds 4-6 mixture+CFA (positive control), 7-9 mixture, and multi-epitope-conjugate 14. The dosage was calculated based on molecular weight to deliver same amount of antigen in each group. Negative control group was treated with PBS. All the vaccine conjugates were able to induce similar level of J8-, PL1-, and 88/30-specific IgG immune responses after final boost (FIG. 24). As expected, the CFA adjuvanted peptide antigens mixture induced the most significant antibody titers between the tested groups, even after primary immunisation. The additional boosts were needed for Compound 14 and mixture 7-9, where the antibody titers were only visible after the second boost. Overall, the physical mixture was able to elicit slightly higher antibody titers than the multiepitope-conjugate (14). However, the difference was not significant. This indicated that the complicated synthesis of Compound 14 was unnecessary to induce an immune response against targeted antigens.

TABLE 2b
Subcutaneous immunization vaccine dosage per mouse.

| | | Dose Per Mouse | | |
|---|---|---|---|---|
| Group | Compound | Compound | | PBS |
| G1 | PBS | — | | 50 μL |
| G2 | CFA | 25 | μL | — |
| | Compound 4 | 41.82 | μg | 25 μL |
| | Compound 5 | 31.47 | μg | |
| | Compound 6 | 31.34 | μg | |
| G3 | Compound 7 | 50.00 | μg | 50 μL |
| | Compound 8 | 39.65 | μg | |
| | Compound 9 | 39.51 | μg | |
| G4 | Compound 14 | 94.36 | μg | 50 μL |

Discussion

The idea behind developing the self-assembling peptides used in this study was to create vaccine candidates that elicited an effective immune response against GAS—with very simple design and ease of synthesis. The compounds were made using minimum components necessary for a successful vaccine—B cell epitope, T cell epitope and adjuvant, to account for specific antibody production, memory T cell production and a delivery system respectively. The minimalistic design helps avoid most of the disadvantages associated with whole organism and whole protein vaccines such as chances of inducing autoimmune diseases, allergic and/or pyrogenic responses, and human cross-tissue reactions. The previously reported data using poly-leucine as pHAAs moiety served as a benchmark for this study. Poly-leucine with 15 amino acids repeat showed better adjuvating ability when compared to other pHAAs, as well as poly-leucine with 10 and 15 residues. However, in this study, due to the hydrophobicity nature of 88/30 and PL1 peptides (more hydrophobic than J8), poly-leucine with only 10 amino acids repeat were used as the pHAAs moiety. The pHAAs were conjugated to the peptide antigen to assess the adjuvanting properties of this delivery system. The pHAA-antigen conjugates were designed to examine the influence of structural parameter on their ability to form nanoparticles and stimulate immune responses. Thus, multi-epitopes conjugate 14 was compared with the physical mixture of Compounds 7-9.

All the compounds were successfully synthesised and purified (purity >95%). Broad peaks were observed in analytical RP-HPLC due to the high hydrophobicity contributed by the poly-leucine tail. The purified Compound 6, in particular, showed three peaks, and all of them matched the expected mass of Ac-PADRE-8830. The 88/30 epitope has a proline on its C terminus, the multiple peaks might be a result of proline existing as its two different isomers, cis and trans in solution. A multiple peak was not observed for Compound 9 (also containing the 88/30 epitope) due to the presence of ten leucine residues, which increased the hydrophobicity of the compound significantly and as a result, a broad signal was observed.

Interestingly, DLS showed aggregate size of 350 nm for Compound 14, which was smaller than the 7-9 mixture (600 nm). The morphology of the mixture of Compounds 7-9 and Compound 14 in TEM is needed to see whether the aggregates measured shows the formation of CLAN. Immunological evaluation of Compound 14 and mixture 7-9 demonstrated that these conjugates were able to induce the production of IgG antibodies against all the antigens (J8, PL1 and 88/30). Physical mixture of polyleucine conjugates with different epitopes induced similar or higher level of antibody titer compared to multiepitope-conjugate 14, which was expected to be more effective due to the incorporation of all the vaccine components in single construct. These results may be correlated to the structure of mixture 7-9 and Compound 14, which need to be evaluated further.

Particles in the nanoparticle range i.e. 1-1000 nm are preferred for targeted delivery as they can easily permeate biological barriers and travel through the body post-administration. However, there is an ongoing debate about the ideal particle size for vaccines. It has been reported that for enhanced uptake by dendritic cells a particle size of less than 500 nm is preferred. Another study reports that particles of size below 200 nm are usually taken up by APCs through endocytosis, resulting in a cellular-based immune response, whereas particles with sizes between 500 nm and 5000 nm are taken up by phagocytosis or micropinocytosis and help promote humoral immunity (Oyewumi and others, 2010). The ideal size ranges for vaccines are also dependent on the antigen, dose and material used to prepare the particles.

Conclusion

This study shows synthesis, characterisation, and immunological evaluation of vaccine candidates against GAS, which incorporate antigenic peptide J8, PL1 and 88/30, T helper cell epitope PADRE and a poly-leucine delivery system. Physical mixture of poly-leucine conjugates with different epitopes induced similar or higher level of antibody titer compared to multiepitope-conjugates, which was expected to be more effective due to the incorporation of all the vaccine components in single construct.

### Example 5

Summary

Peptide antigens have been widely used in the development of vaccines, especially for those against autoimmunity-inducing pathogens and cancers. However, peptide-based vaccines require adjuvant and/or a delivery system to stimulate desired immune responses. Here, we explored the potential of self-adjuvanting poly(hydrophobic amino acids) (pHAAs) to deliver peptide-based vaccine against Group A Streptococcus (GAS). We designed and synthesized self-assembled nanoparticles with a variety of conjugates bearing peptide antigen (J8-PADRE) and polymerized hydrophobic amino acids to evaluate the effects of structural arrangement and pHAAs properties on a system's ability to induce humoral immune responses. Immunogenicity of the developed conjugates was also compared to commercially available human adjuvants. We found that a linear conjugate bearing J8-PADRE and 15 copies of leucine induced equally effective, or greater, immune responses than commercial adjuvants. Our fully defined, adjuvant-free, single molecule-based vaccine induced the production of antibodies capable of killing GAS bacteria.

Introduction

Vaccine discovery has been regarded as an important milestone in human history, as vaccination is the most effective strategy for controlling and preventing infectious diseases [1, 2] In-line with developments in molecular biology, chemistry and immunology, vaccines have undergone significant changes over the past 200 years. Instead of relying on attenuated or killed whole organisms, current research focuses more on the development of subunit vaccines, which are highly purified and safer. Subunit vaccines only contain the essential antigenic parts of a pathogen (e.g. protein, peptides, carbohydrates, etc.), and as such, contain no, or diminished, biological impurities and have a low chance of inducing allergic or autoimmune responses [3, 4]. However, the use of minimal antigen components also means these vaccines are less immunogenic. In order to counter this problem, adjuvants have been added as complementary immunostimulants to these vaccine formulations. Currently, all commercial subunit vaccines utilize adjuvants and/or delivery systems for improved efficacy [5-7].

Adjuvants mimic natural pathogen-associated danger signals, in order to gain recognition by the human innate immune system and boost and/or modify specific adaptive immune responses when co-administered with vaccine antigens. Although a few adjuvants are commercially available for vaccine design, most of them have some level of toxicity or can induce side-effects, such as local adverse reactions at the injection site (e.g. inflammation, redness, swelling, pain) or systemic reactions (e.g. malaise, fever, adjuvant arthritis, anterior uveitis) [5, 8]. Moreover, these adjuvants are limited in application because of the complexity of the preparation process and high costs. Therefore, attention has shifted towards a new generation of adjuvants: nano-adjuvants, as nanomaterials are generally safe, easy to synthesize and modify, can be chemically defined, and have high immunostimulating capacity [9, 10].

Nanoparticles formed from self-assembled amphiphiles have been shown to have promising self-adjuvanting properties [9, 11] and are often used in peptide vaccine delivery [12-14]. For example, hydrophobic dendritic poly(tert-butyl acrylate) [15] or lipoamino acids (e.g. 2-amino-D,L-hexadecanoic acid) [16] have been conjugated to a variety of peptide-based antigens and the produced conjugates were self-assembled into micro/nanoparticles [17, 18]. These particles stimulated strong immune responses, both humoral and cellular, against incorporated antigens [19-24]. However, these compounds, especially polyacrylates, had fundamental disadvantageous for potential commercial application as they are racemic, not biodegradable, and have undefined number of units (in case of the polymer). Thus, the batch-to-batch variability may affect in vitro and in vivo vaccine efficacy and make them unsuitable for clinical trials.

In recent studies, we introduced poly(hydrophobic amino acid) (pHAA), as a fully-defined and effective self-adjuvating delivery system for peptide antigens [25]. When conjugated to hydrophilic peptide antigen, pHAA sequences serve as self-adjuvating moieties that enable self-assembly of the pHAA-antigen conjugate into small nanoparticles and chain-like aggregates of nanoparticles (CLANs). The conjugation of Group A Streptococcus (GAS) M protein-derived antigen 1 to polyleucine produced conjugate 2 (FIG. 26), which self-assembled into CLANs and induced the production of a high level of opsonic antibodies in mice.

The importance of spatial arrangement in peptide vaccine components on the quality of immune responses has been reported previously [26-30]. Thus, to optimize the adjuvanting capacity of pHAAs, we designed vaccine constructs composed of (i) conserved B-cell epitope J8 (QAEDKVKQSREAKKQVEKALKQLEDKVQ; SEQ ID NO:9) derived from GAS M protein [31], (ii) universal T-helper cell epitope (pan HLA DR-binding epitope, PADRE: AKFVAAWTLKAAA; SEQ ID NO:7) [32], and (iii) polyleucine moieties conjugated in different spatial arrangements (FIG. 26 b). PADRE is commonly used to enhance the quality and longevity of humoral immune responses [32]. Both J8 and PADRE have been tested in clinical trials and have consistent safety profiles [33, 34]. Variation in pHAA type was also investigated to further optimize the adjuvanting activity of the pHAA moieties (FIG. 26 c). The vaccine candidates were then compared with commercially available adjuvants to evaluate their efficacy.

Materials and Methods

Boc-protected 1-amino acids were purchased from Novabiochem, Merck Chemicals (Darmstadt, Germany) and Mimotopes (Melbourne, Australia). 1-[6-Bis(dimethylamino)methylene]-1H-1, 2, 3-triazolo[4, 5-b]pyridinium-3-oxide hexafluorophosphate (HATU) was also purchased from Mimotopes. Acetonitrile, dichloromethane (DCM), methanol, N,N-dimethylformamide (DMF), N,N-diisopropylethylamine (DIPEA), piperidine, rink-amide methylbenzhydrylamine (MBHA) resin, trifluoroacetic acid (TFA), and phenylmethanesulfonyl fluoride (PMSF) were acquired from Merck (Hohenbrunn, Germany). Rink amide p-methyl-benzhydrylamine hydrochloride (pMBHA.HCl) resin (substitution: 0.59 mmol/g; 100-200 mesh) was obtained from Peptides International (Kentucky, USA). PBS was obtained from eBioscience (California, USA). C57BL/6 mice were purchased from The University of Queensland Biological Resources (UQBR) (Queensland, Australia). Imject™ alum adjuvant (aqueous aluminum hydroxide and magnesium hydroxide) was purchased from Thermo Scientific (Victoria, Australia). Goat anti-mouse IgG (H+L)-horseradish peroxidase (IgG-HRP) conjugate was purchased from Millipore, Temacula (California, USA). AddaVax™ adjuvant (MF59; squalene-based oil-in-water nano-emulsion), CFA (heat-killed Mycobacterium tuberculosis in non-metabolizable oils (paraffin oil and mannide monooleate)), MPLA-SM VacciGrade™ adjuvant (AS04; monophosphoryl lipid A derivative from Salmonella minnesota R595 lipopolysaccharide), and goat anti-mouse IgA cross-adsorbed secondary antibody HRP (IgA-HRP) conjugate were purchased from Invivogen (San Diego, USA). Analytical-grade Tween 20 was purchased from VWR International (Queensland, Australia). Chloroform, ethylenediaminetetraacetic acid (EDTA), o-phenylenediamine dihydrochloride (OPD) substrate, pilocarpine hydrochloride and all other reagents were purchased from Sigma-Aldrich (Victoria, Australia).

Synthesis of Compounds 1-11. All vaccine candidates were synthesized using microwave-assisted Boc-SPPS (70° C., 20 W) performed on an SPS CEM Discovery reactor from CME Corporation (North Carolina, USA) at 0.1 mmol scales, following the previously reported method [49]. Resin (0.170 g) was weighted and pre-swelled for 2 h in DMF and DIPEA (6.2 equiv.). The coupling cycle included deprotection of the Boc group (2 min treatment with neat TFA, twice at RT), DMF wash, amino acid (0.84 mmol/g, 4.2 equiv.) activation with 0.5M HATU (1.6 mL, 4 equiv.) and DIPEA (0.18 mL, 6.2 equiv.), and its double-coupling to the resin (5 and 10 min). Aspartic acid, however, was double-coupled for 15 min at 50° C., and Boc-Gln(Xan)-OH was deprotected using DCM between TFA treatment (instead of DMF) to avoid cyclization of glutamine. These procedures were repeated until the desired peptides were achieved. Acetylation was performed after the addition of the final amino acid using acetylation solution (90% DMF, 5% DIPEA and 5% acetic anhydride). The formyl group from tryptophan was then removed using 20% piperidine in DMF for 2 min, then 5 min. The produced peptide resin was washed with DMF (3×), DCM (3×) and methanol (lx) before being dried in a desiccator overnight.

After drying, the peptides were cleaved from the resin using anhydrous hydrogen fluoride (HF) and p-cresol as a scavenger. The peptides, 1-11, were precipitated and washed with cold diethyl ether or cold diethyl ether:n-hexane (1:1) for hydrophilic and hydrophobic compounds, respectively. The precipitated compounds were then dissolved using solvent A (100% milli-Q water, 0.1% TFA) and solvent B (90% acetonitrile, 10% milli-Q water, 0.1% TFA) at a ratio of 1:1, or pure solvent A or B for hydrophilic and hydrophobic compounds, respectively, then filtered. The compounds were purified using a Shimadzu preparative reverse-phase HPLC (RP-HPLC; Kyoto, Japan) instrument (LC-20AP×2, CBM-20A, SPD-20A, FRC-10A) with a 20.0 mL/min flow rate on a C18 (218TP1022; 10 μm, 22×250 mm), C8 (208TP54; 5 m, 4.6×250 mm) or C4 (214TP1022; 10 μm, 22×250 mm) column with solvent B gradients (specific gradients for each compound) for 25 min, with compound detection at 214 nm. The purity of all compounds (>95%) were determined using analytical RP-HPLC on a C18 (218TP54; 5 μm, 4.6×250 mm), C8 (208TP54; 5 m, 4.6×250 mm) or C4 (214TP54; 5 μm, 4.6×250 mm) Vydac column, with a 0-100% gradient of solvent B for 40 min and compound detection at 214 nm. ESI-MS was performed on a LCMS-2020 Shimadzu (Kyoto, Japan) instrument (DGU-20A3, LC-20Ad×2, SIL-20AHT, STO-20A) and Analyst 1.4 software (Applied Biosystems/MDS Sciex, Toronto, Canada) (Perkin-Elmer-Sciex API3000). Analytical RP-HPLC was performed on a Shimadzu (Kyoto, Japan) instrument (DGU-20A5, LC-20AB, SIL-20ACHT, SPD-M10AVP).

Compound 1 yield: 18%. Purity: 99%. Molecular weight: 4653.42 g/mol. ESI-MS: [M+3H]³⁺ m/z 1552.4 (calcd: 1552.1), [M+4H]⁴⁺ m/z 1164.3 (calcd: 1164.4), [M+5H]⁵⁺ m/z 931.7 (calcd: 931.7), [M+6H]⁶⁺ m/z 776.6 (calcd: 776.6), [M+7H]⁷⁺ m/z 665.9 (calcd: 665.8). Rₜ: 25.0 min (0-100% solvent B, 40 min, C18 column).

Compound 2 yield: 18%. Purity: 98%: Molecular weight: 6521.03 g/mol. ESI-MS: [M+4H]⁴⁺ m/z 1631.0 (calcd: 1631.3), [M+5H]⁵⁺ m/z 1305.3 (calcd: 1305.2), [M+6H]⁶⁺ m/z 1087.6 (calcd: 1087.8), [M+7H]⁷⁺ m/z 932.5 (calcd: 932.6), [M+8H]⁸⁺ m/z 816.2 (calcd: 816.1). Rₜ: 26.9 min (0-100% solvent B, 40 min, C4 column).

Compound 3 yield: 20%. Purity: 98%. Molecular weight: 6479.00 g/mol. ESI-MS: [M+4H]⁴⁺ m/z 1620.7 (calcd: 1620.8), [M+5H]⁵⁺ m/z 1296.9 (calcd: 1296.8), [M+6H]⁶⁺ m/z 1081.0 (calcd: 1080.8), [M+7H]⁷⁺ m/z 926.6 (calcd: 926.6), [M+8H]⁸⁺ m/z 810.9 (calcd: 810.9), [M+9H]⁹⁺ m/z 721.4 (calcd: 720.9). Rₜ: 38.2 min (0-100% solvent B, 40 min. C4 column).

Compound 4 yield: 16%. Purity: 98%. Molecular weight: 6479.00 g/mol. ESI-MS: [M+4H]⁴⁺ m/z 1620.5 (calcd: 1620.8), [M+5H]⁵⁺ m/z 1296.9 (calcd: 1296.8), [M+6H]⁶⁺ m/z 1081.0 (calcd: 1080.8), [M+7H]⁷⁺ m/z 926.6 (calcd: 926.6), [M+8H]⁸⁺ m/z 811.2 (calcd: 810.9), [M+9H]⁹⁺ m/z 721.5 (calcd: 720.9). Rₜ: 29.1 min (0-100% solvent B, 40 min, C4 column).

Compound 5 yield: 17%. Purity: 97%. Molecular weight: 6350.82 g/mol. ESI-MS: [M+5H]⁵⁺ m/z 1272.0 (calcd: 1271.2), [M+6H]⁶⁺ m/z 1060.0 (calcd: 1059.5), [M+7H]⁷⁺ m/z 909.0 (calcd: 908.3), [M+8H]⁸⁺ m/z 795.0 (calcd: 794.9), [M+9H]⁹⁺ m/z 707.0 (calcd: 706.7), [M+10H]¹⁰⁺ m/z 636.0 (calcd: 636.1). Rₜ: 38.6 min (0-100% solvent B, 40 min, C4 column).

Compound 6 yield: 13%. Purity: 95%. Molecular weight: 5509.20 g/mol. ESI-MS: [M+4H]⁴⁺ m/z 1379.0 (calcd: 1378.3), [M+5H]⁵⁺ m/z 1103.0 (calcd: 1102.8), [M+6H]⁶⁺ m/z 919.0 (calcd: 919.2), [M+7H]⁷⁺ m/z 788.0 (calcd: 788.0), [M+8H]⁸⁺ m/z 690.0 (calcd: 689.7). Rₜ: 24.0 min (0-100% solvent B, 40 min, C4 column).

Compound 7 yield: 10%. Purity: 96%. Molecular weight: 5719.61 g/mol. ESI-MS: [M+5H]⁵⁺ m/z 1145.0 (calcd: 1144.9), [M+6H]⁶⁺ m/z 955.0 (calcd: 954.3), [M+7H]⁷⁺ m/z 818.0 (calcd: 818.1), [M+8H]⁸⁺ m/z 716.0 (calcd: 716.0), [M+9H]⁹⁺ m/z 637.0 (calcd: 636.5). Rₜ: 24.9 min (0-100% solvent B, 40 min, C4 column).

Compound 8 yield: 18%. Purity: 99%. Molecular weight: 6110.18 g/mol. ESI-MS: [M+5H]⁵⁺ m/z 1223.0 (calcd: 1223.0), [M+6H]⁶⁺ m/z 1019.0 (calcd: 1019.4), [M+7H]⁷⁺ m/z 874.0 (calcd: 873.9), [M+8H]⁸⁺ m/z 765.0 (calcd: 764.8), [M+9H]⁹⁺ m/z 680.0 (calcd: 679.9), [M+10H]¹⁰⁺ m/z 612.0 (calcd: 612.0). Rₜ: 22.4 min (0-100% solvent B, 40 min, C4 column).

Compound 9 yield: 20%. Purity: 99%. Molecular weight: 6590.15 g/mol. ESI-MS: [M+4H]⁴⁺ m/z 1648.5 (calcd: 1648.5), [M+5H]⁵⁺ m/z 1319.2 (calcd: 1319.0), [M+6H]⁶⁺ m/z 1099.4 (calcd: 1099.4), [M+7H]⁷⁺ m/z 942.5 (calcd: 942.5), [M+8H]⁸⁺ m/z 824.9 (calcd: 824.8). Rₜ: 18.7 min (0-100% solvent B, 40 min, C4 column).

Compound 10 yield: 16%. Purity: 96%. Molecular weight: 5959.59 g/mol. ESI-MS: [M+5H]⁵⁺ m/z 1193.0 (calcd: 1192.9), [M+6H]⁶⁺ m/z 995.0 (calcd: 994.3), [M+7H]⁷⁺ m/z 853.0 (calcd: 852.4), [M+8H]⁸⁺ m/z 746.0 (calcd: 746.0), [M+9H]⁹⁺ m/z 663.0 (calcd: 663.2). Rₜ: 23.9 min (0-100% solvent B, 40 min, C4 column).

Compound 11 yield: 19%. Purity: 97%. Molecular weight: 6310.50 g/mol. ESI-MS: [M+5H]⁵⁺ m/z 1263.0 (calcd: 1263.1), [M+6H]⁶⁺ m/z 1053.0 (calcd: 1052.8), [M+7H]⁷⁺ m/z 903.0 (calcd: 902.5), [M+8H]⁸⁺ m/z 790.0 (calcd: 789.8), [M+9H]⁹⁺ m/z 703.0 (calcd: 702.2). Rₜ: 45.5 min (0-100% solvent B, 40 min, C8 column).

Dynamic Light Scattering. Individual compounds 1-4 (1 mg/mL in PBS) and 5-11 (2 mg/mL in PBS) were analyzed using DLS to measure particle size (zeta intensity) and PDI. The samples were treated with ultrasonication for 30 min, then transferred into disposable cuvettes. Measurement were taken at 25° C. and 1730 light scattering. DLS measurements were taken using a Zetasizer Nano ZP instrument (Malvern Instrument, UK) with Malvern Zetasizer Analyser 6.2 software.

Transmission Electron Microscopy. Particle-imaging was captured using a JEM-1010 TEM (HT7700 Exalens, HITACHI Ltd., JEOL Ltd., Japan) operated at 80 kV, using negative staining. Samples of compounds 1-11 (1:2 dilution of samples prepared for DLS analysis) were applied to glow-discharged carbon-coated copper 200-mesh grids (Ted Pella) and negative-stained with 2% phosphotungstic acid.

Secondary Structure Analysis. The secondary structure of peptides was analyzed in a 1 mm cuvette using a Jasco J-710 CD Spectrometer (Jasco Corp., Japan). Compounds 1, and 5-11 (0.2 mg/mL in PBS) were placed in quartz cuvettes. CD spectra were measured with 1 mm path length at 23° C., and the data were collected between 195 and 275 nm with a continuous scanning speed of 50 nm/min.

Quantitative analysis of the secondary structure content was measured via the determination method [50] by solving the composite spectrum of a given compound to yield contribution ratios of each pure individual component spectrum, i.e. pure α-helix, β-sheet and random coil contribution, obtained from the reported poly-L-lysine spectra.

Subcutaneous Immunization of Mice. Six-week-old female C57BL/6 mice were housed at the Australian Institute for Bioengineering and Nanotechnology (AIBN) Animal Facility. The mice were allowed to acclimatize to the new environment for 7 days before any experiments were performed. They were then subcutaneously immunized at the tail base with Compounds 2-4 (50 μg in 50 μL of PBS) on day 0, followed by three boosts of the same dose on days 21, 28 and 35 (n=5; FIG. 35; Table 2c). Positive control mice were given Compound 1 emulsified with CFA adjuvant at a 1:1 volume ratio, as per the manufacturer's instructions. Negative control mice received 50 μL of PBS.

TABLE 1c
Physicochemical characterization of the vaccine conjugates.

| | Particle size (nm) | | Polydispersity |
|---|---|---|---|
| Compounds | TEM | DLS^{a)} | index (PDI) |
| 1 | Particles not visible | 4 ± 0 | 0.42 ± 0.10 |
| | | 265 ± 21 | |
| 2 | 10-20 nm NP and CLANs | 210 ± 7 | 0.23 ± 0.03 |
| 3 | 10-20 nm NP and CLANs | 356 ± 17 | 0.23 ± 0.01 |
| 4 | 10-20 nm NP and 100-350 nm | 478 ± 7 | 0.10 ± 0.07 |
| | spherical aggregates | | |
| 5 | 60-100 nm NP and CLANs | 61 ± 4 | 0.21 ± 0.03 |
| | | 293 ± 3 | |
| 6 | 20-30 nm NP, CLANs and | 37 ± 2 | 0.48 ± 0.01 |
| | 100-450 nm elongated rods | 229 ± 9 | |
| | | 4955 ± 193 | |
| 7 | 10-20 nm NP and CLANs | 610 ± 14 | 0.26 ± 0.26 |
| 8 | 20-30 nm NP | 376 ± 17 | 0.33 ± 0.02 |
| 9 | Particles not visible | 6 ± 0.2 | 0.67 ± 0.13 |
| | | 37 ± 22 | |
| | | 233 ± 14 | |
| 10 | 10-90 nm NP and CLANs | 240 ± 9 | 0.31 ± 0.02 |
| 11 | 10-20 nm NP and CLANs | 255 ± 9 | 0.44 ± 0.02 |

^{a)}size as measured by intensity.
NP = nanoparticles; CLANs = chain like aggregates of nanoparticles.

A similar immunization schedule was followed for the second animal study: Compounds 1 and 5-11 (100 μg in 50 μL of PBS) were subcutaneously injected in the tail of mice (n=5; Table 2c). Mice were administered with compound 1 mixed with commercial adjuvants (CFA, alum, MF59 and AS04) at a 1:1 volume ratio as adjuvanted controls; negative control mice received 50 μL of PBS.

TABLE 2c
Subcutaneous immunization vaccine dosage per mouse.

| | | Dose per mouse | |
|---|---|---|---|
| Group | Compound | Compound | PBS |

a) First Animal Study (Influence of epitopes arrangement)

| G1 | PBS | — | | 50 μL |
|---|---|---|---|---|
| G2* | CFA | 25 | μL | — |
| | 1 | 50 | μg | 25 μL |
| G3 | 2 | 50 | μg | 50 μL |
| G4 | 3 | 50 | μg | 50 μL |
| G5 | 4 | 50 | μg | 50 μL |

b) Second Animal Study (Influence of pHAAs types)

| G1 | PBS | — | | 50 μL |
|---|---|---|---|---|
| G2 | 1 | 100 | μg | 25 μL |
| G3* | CFA | 25 | μL | — |
| | 1 | 100 | μg | 25 μL |
| G4 | Alum | 25 | μL | — |
| | 1 | 100 | μg | 25 μL |
| G5 | MF59 | 25 | μL | — |
| | 1 | 100 | μg | 25 μL |
| G6 | AS04 | 5 μg in 5 μL DMSO | | 20 μL |
| | 1 | 100 | μg | 25 μL |
| G7 | 5 | 100 | μg | 50 μL |
| G8 | 6 | 100 | μg | 50 μL |
| G9 | 7 | 100 | μg | 50 μL |
| G10 | 8 | 100 | μg | 50 μL |
| G11 | 9 | 100 | μg | 50 μL |
| G12 | 10 | 100 | μg | 50 μL |
| G13 | 11 | 100 | μg | 50 μL |

*Mice (n = 5) received vaccine formulation containing CFA during primary immunization on day 0, and only compound 1 in 50 μL PBS were injected during the subsequent boosts (day 21, 28 and 35).

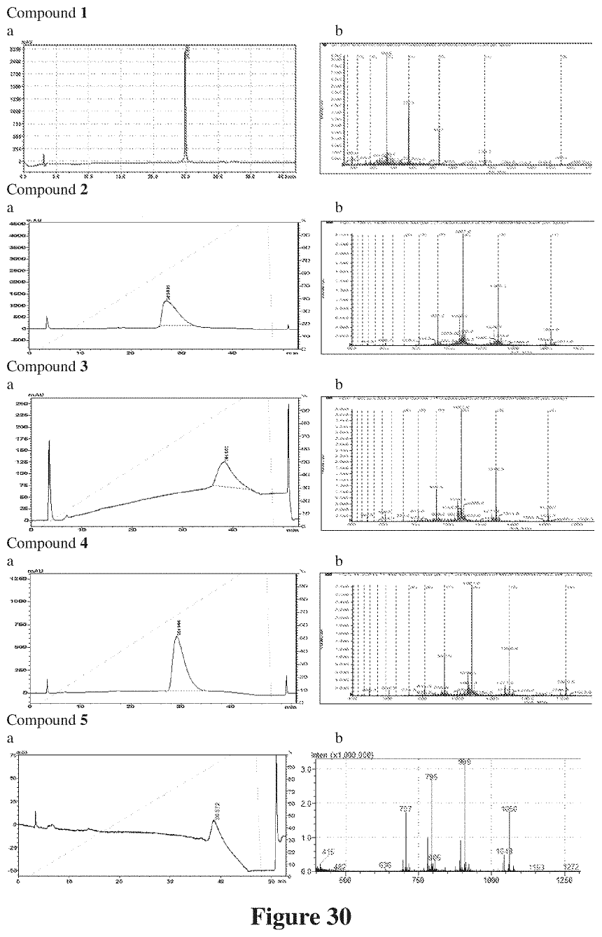

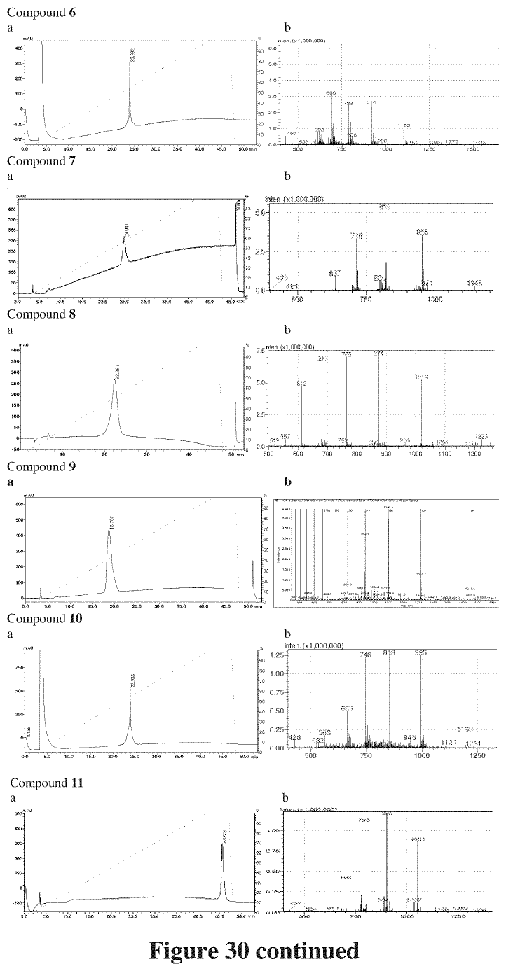

Collection of Serum and Saliva. Serum samples were collected on days −1 (naïve serum), 20, 27, 34, and 49 to measure antigen-specific IgG antibody titers (FIG. 30). Blood samples were collected from the tail tip (10 μL diluted in 90 μL of PBS on days 0, 20, 27 and 34) and heart puncture (1 mL blood collected on day 49). The samples were centrifuged at 3,600 rpm for 10 min. Serum was collected from the supernatant, and kept at −80° C. until further analysis.

Saliva samples were collected on days −1 (naïve saliva) and 42 to measure J8-specific IgA and IgG antibody titers. Salivation was induced by administering 50 μL of 0.1% pilocarpine intraperitoneally to the mice. 100 μL samples of saliva were collected into 2 μL of protease inhibitor in 100 mM PMSF solution (100 mM, 17.4 mg PMSF in 1 mL ethanol). Saliva samples were kept at −80° C. until further analysis.

Antibody Titer Detection by ELISA. Antigen-specific antibody IgG and IgA titers from serum and saliva samples, respectively, were detected using enzyme-linked immunosorbent assay (ELISA). A 96-well plate was coated with 50 μg of antigen J8 or p145 (LRRDLDASREAKKQVEKALE; SEQ ID NO:40). A two-fold serial dilution was performed on the antigen-coated plate using 0.5% skim milk, starting at a 1:100 concentration of serum and a 1:2 concentration of saliva. Naïve mice sera and saliva (day −1 samples) were used as controls. Diluted secondary antibody (1:3000 IgG-HRP for serum and 1:1000 IgG- or IgA-HRP for saliva) was added to the plate before adding OPD substrate. A SpectraMax microplate reader (Molecular Devices, USA) was used to read the absorbance at 450 nm. The antibody titers were expressed as the lowest possible dilution with absorbance of more than three times the standard deviation above the mean absorbance of control wells.

The data were analyzed using GraphPad Prism 7 software (GraphPad Software, Inc., USA). Multiple comparisons were performed using one-way ANOVA with Tukey's multiple comparison test. Data were considered significantly different at (*) p<0.05, (**) p<0.01, (***) p<0.001, and (****) p<0.0001 among the studied group.

Antibody Opsonization Assay. Opsonization assays were performed as previously published [39], using D3840 and GC2 203 GAS clinical isolates. Bacteria were prepared by streaking on Todd Hewitt Broth (THB) agar plates (supplemented with 5% yeast extract), then incubated at 37° C. for 24 h. A single colony from each bacterium was transferred to 5 mL of THB (supplemented with 5% yeast extract) and incubated at 37° C. for 24 h to replicate the bacteria to approximately 4.6×10⁶ colony forming units (CFU)/mL. The cultures were serially diluted (two-fold) in PBS with a 10 μL aliquot mixed with 10 μL heat-inactivated serum collected on day 49, together with 80 μL horse blood. Serum samples were inactivated by heating in a 50° C. water bath for 30 min. The assays were performed in duplicate from two independent cultures. Bacteria were incubated in a 96-well plate at 37° C. for 3 h in the presence of the serum, before 10 μL of the aliquot was plated on THB agar plates (supplemented with 5% yeast extract and 5% horse blood) and incubated at 37° C. for 24 h. The bacterial survival rate was analyzed based on CFU enumerated from the plates. The opsonic activity (% reduction in mean CFU) of the antibody sera was calculated as: [(1-CFU in presence of antibodies sera mean)/CFU in presence of PBS]×100%.

Results

Preparation of Vaccine Candidates. The ability of pHAAs conjugated to antigen 1 to induce humoral immune responses against GAS was recently reported [25]: Compound 2 was able to induce highly opsonic antigen-specific antibody production in mice upon subcutaneous immunization. To examine the structure-activity relationship of pHAA-antigen conjugates, new conjugates were designed. GAS B-cell epitope J8 conjugated to PADRE universal T-helper (Compound 1) was modified with 15 copies of leucine; however, the arrangement of epitopes was modified in comparison to parent Compound 2 (FIG. 26 b). Namely, the structure was simplified by linear rearrangement of the epitopes to form Compounds 3 and 4. Compounds 5-10 were designed to incorporate PADRE and J8 epitopes conjugated with different types of pHAAs to analyze the adjuvating ability of these polymeric units (FIG. 26 c). Compounds 1-11 were synthesized using microwave assisted butyloxycarbonyl (Boc) solid-phase peptide synthesis (SPPS), purified using preparative reverse-phase high performance liquid chromatography (RP-HPLC), and their structure and purity (>95%) were confirmed using electrospray ionization mass spectrometry (ESI-MS) and analytical RP-HPLC (FIG. 30).

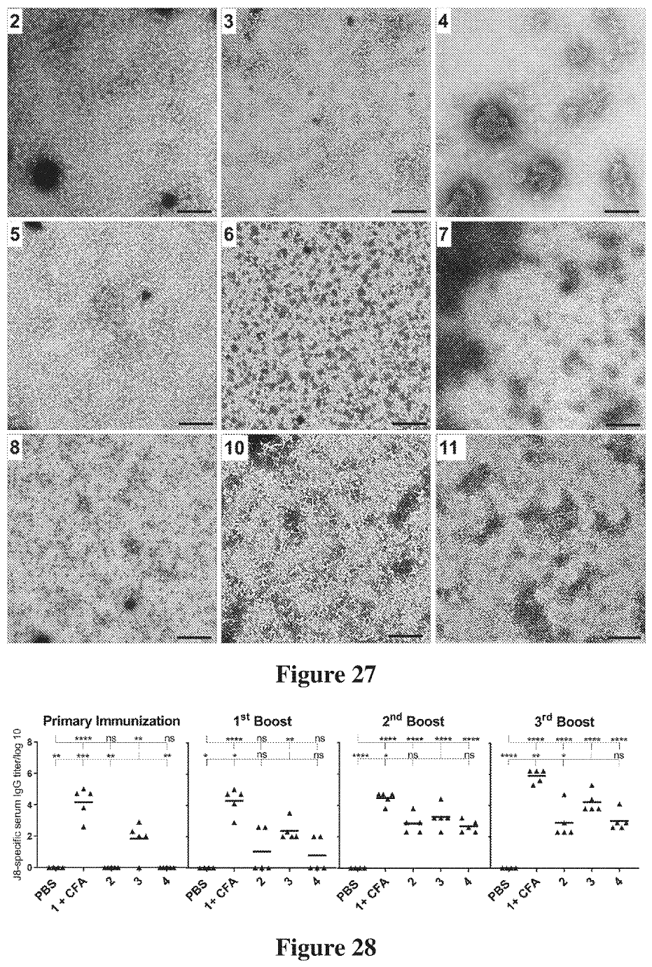

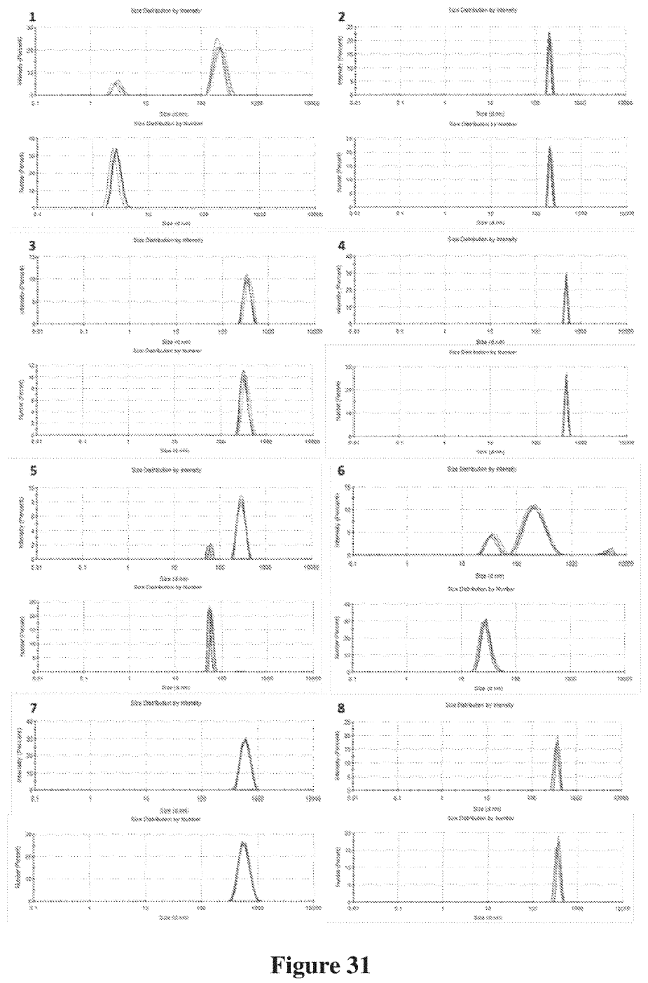

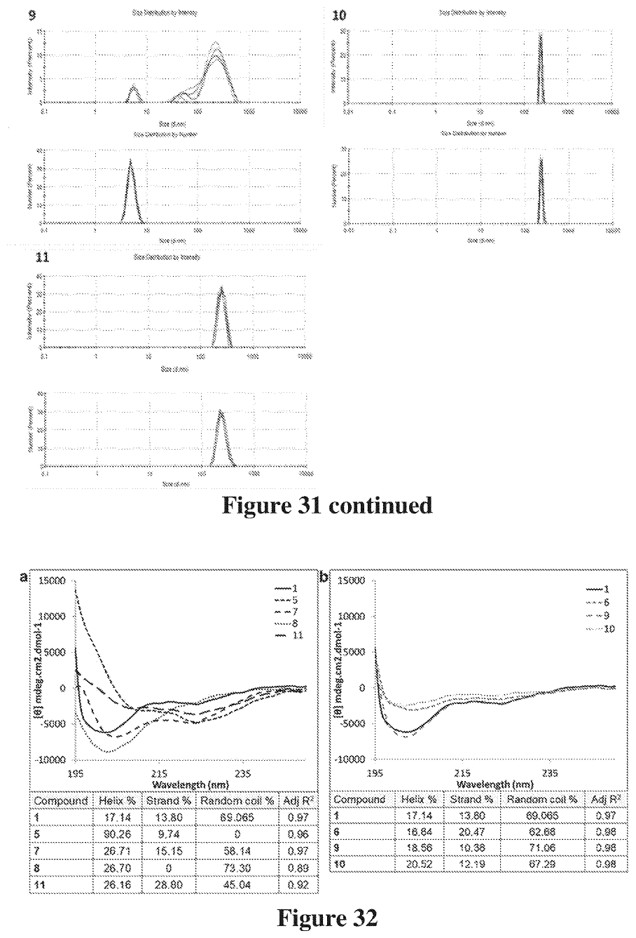

Nanoparticle Formation and Analysis. Individual Compounds 1-11 were self-assembled to form nanoparticles in phosphate-buffered saline (PBS). The compounds' secondary structure, particle size (zeta intensity), size distribution (PDI), and morphology were characterized using circular dichroism (CD) spectroscopy, dynamic light scattering (DLS) and transmission electron microscope (TEM; FIG. 27; Table 1c; FIGS. 31 and 32). The predisposition of peptides containing leucine to adopt a helical conformation has been suggested [35]. Peptide antigen 1 adopted a mostly random coil conformation on its own, but became helical once it was modified with polyleucine (5) [25]. However, none of the other pHAAs, as in Compounds 6-11, were able to significantly shift the overall random coil conformation of 1 toward α-helix. Polyglutamic acid (9), in particular, had limited to no effect on the antigen secondary structure, followed by polyglycine (6) and polyserine (10) (FIG. 32). Alanine (7) and phenylalanine-leucine-alanine (11)-based conjugates adopted practically the same conformation as 1.

Compounds 1-11 were self-assembled into nanoparticles by simple mixing (vortexing) with PBS. In general, and comparable to what has been shown previously for Compound 2 [25], most of the compounds formed a mixture of nanoparticles and aggregates. Interestingly, DLS analysis of peptide 1 also detected the presence of particles; mainly the peptide itself or small aggregates (4 nm), and submicron sized aggregates (300 nm) (Table 1c; FIG. 31). However, no particles were visible through TEM analysis (data not shown). Branched Compound 2 formed evenly distributed small nanoparticles (10-20 nm) with visible aggregates resembling CLANs (according to TEM; FIG. 27), similar to what was seen previously [25]. Epitope rearrangement into a linear structure affected conjugate self-assembly to a degree. Linear Compound 3 formed similar CLANs to branched Compound 2, while Compound 4 self-assembled into classical globular aggregates of nanoparticles 100-350 nm in size, according to TEM images (FIG. 27), and 500 nm according to DLS analysis (Table 1c; FIG. 31). Compound 5 was synthesized as an analogue of Compound 3, with the redundant branching lysine between PADRE and J8 eliminated. Deletion of the lysine did not affect the particle size, and identical CLANs were formed compared to Compound 3. In contrast, the replacement of polyleucine in 5 with polyglycine (6) resulted in the formation of extended rods (or short fibrils) alongside of the nanoparticles (20-30 nm), while polyalanine (7) self-assembled into nanoparticle aggregates with less apparent CLANs visible through TEM. Self-assembly of polyproline-conjugate 8 induced the formation of small nanoparticles (20-30 nm) according to TEM, which were detected rather as aggregates by DLS (400 nm). Conjugation of poly-glutamic acid to peptide antigen 1 to form Compound 9 did not change the water solubility or polydispersity of the peptide antigen. DLS analysis of Compound 9 detected what was most likely the peptide itself (6 nm) with aggregates (40 and 230 nm), which were not visible in TEM (data not shown). Self-assembly of compound 10 containing poly-serine and 5 repeats of phenylalanine-leucine-alanine pHAAs (11) formed similar CLANs to 5. Overall, Compounds 5-11 formed small nanoparticles, which aggregated.

Immunological Evaluation of the Conjugates. Immunological evaluation of the polyHAA-antigen conjugates was performed with C57BL/6 mice using the prime-boost vaccination strategy. Negative control mice were treated with PBS.

Conjugates 2-4 (Influence of Epitope Arrangement). Mice were subcutaneously immunized with Compound 1+complete Freund's adjuvant (CFA; positive control) and individual Compounds 2-4. Conjugate 3 induced significant immunoglobulin G (IgG) titers after the primary immunization, and all vaccine conjugates were able to induce the production of J8-specific IgG antibodies after the final boost (FIG. 28). Conjugate 3 generated higher antibody titers than the previously identified lead vaccine candidate (2) [25], following each immunization. There were no significant differences between the antibody titers induced by Compounds 2 and 4.

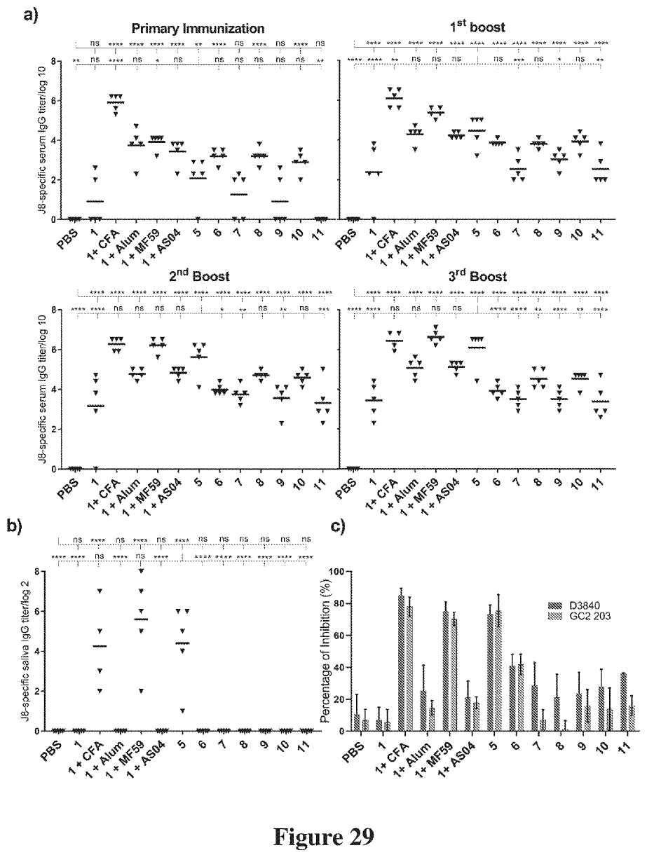

Conjugates 5-11 (Influence of pHAA Type). Mice were subcutaneously immunized with peptide epitope 1, 1 combined with different adjuvants (CFA, Imject™ (alum), AddaVax™ (MF59), and MPLA-SM VacciGrade™ (ASO4)) as adjuvanted controls, and individual Compounds 5-11. The serum and saliva samples were analyzed for the presence of antibodies against J8 and native M protein fragment p145, and for their ability to opsonize GAS. All of the vaccine conjugates were able to induce significant J8-specific IgG immune responses, even after the first boost (FIG. 29 a). Compound 1 adjuvanted with human-grade commercial adjuvants (alum, MF59 and AS04) triggered the production of significant J8-specific IgG titers after primary immunization. Laboratory ‘gold-standard’ CFA adjuvant was even more potent; however, it also caused toxic side-effects resulting in one dead mouse. Compound 5 induced significantly higher IgG antibody titers than any of the other pHAA conjugates after the 3^{rd} boost. Antibody production induced by Compound 5 after the first boost was weaker than CFA-adjuvanted antigen, but was comparable to the other commercial adjuvants. After the 2^{nd} boost, the antibody titers induced by Compound 5 were similar to CFA and MF59, and higher than alum and AS04. Compounds 6-11 were less effective in inducing J8-specific IgG immune responses, and these responses were not significantly higher than those induced by antigen 1 itself, even after the 3^{rd} boost.

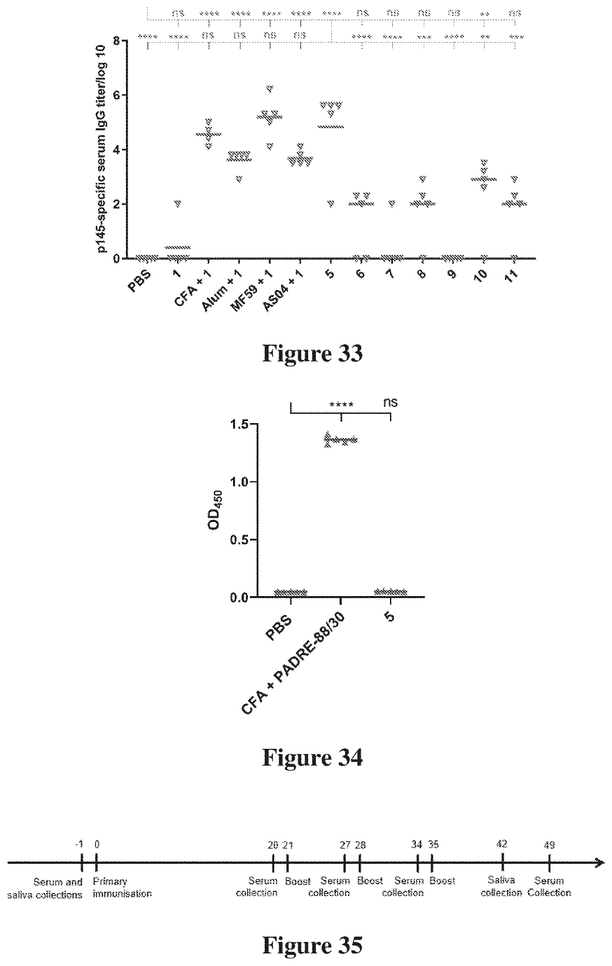

The production of J8-specific IgG antibodies in saliva was also tested. Salivary IgG was only present in mice immunized with 1+CFA, 1+MF59 and 5 (FIG. 29 b). In contrast, none of the vaccine candidates or the control were able to induce the production of J8-specific immunoglobulin A (IgA) antibodies in saliva upon subcutaneous immunization (data not shown). The ability of antibodies produced by mice immunized with Compounds 5-11 to recognize native fragments of M protein (p145) was also examined (FIG. 33). Compound 5 and the adjuvanted Compound 1 were able to induce the production of significant p145-specific IgG antibodies (p<0.0001); whereas the antibodies produced following immunization with Compounds 6-11 and antigen 1 alone were less efficient or completely unable to recognize p145. Finally, to further examine the quality of the produced antibodies, bactericidal assays were performed (FIG. 29 c). Only the sera of mice immunized with 1+CFA, 1+MF59 and 5 had the ability to kill GAS bacteria.

Discussion

GAS is one of the most detrimental human pathogens. It can invade any part of the body and is responsible for a wide variety of human diseases, ranging from mild infections (e.g. strep throat, scarlet fever, impetigo) to invasive infection (e.g. necrotizing fasciitis, toxic shock syndrome) and deadly post-streptococcal sequelae (e.g. acute rheumatic fever, rheumatic heat disease, acute glomerulonephritis, bacteremia, pneumonia) [36]. The M protein is a major virulent factor of GAS and the main target for vaccine development. However, it is also considered to be one of the main factors responsible for the induction of autoimmune responses during GAS infection. Therefore, GAS vaccine development has focused on peptide antigens [36]. Indeed, all GAS vaccine candidates that have reached clinical trials are based on M protein-derived peptide antigens. Fragments of the M protein-conserved C-repeat region, p145 peptide, were initially used for GAS vaccine design. However, the epitope, due to its partial similarity to human heart tissue [31], was later modified into chimeric J8 antigen, and this has since been demonstrated to be safe in clinical trials [33]. The vaccine was designed as a conjugate between diphtheria toxoid carrier protein and J8, adjuvanted with alum (Alhydrogel, 2% aluminum hydroxide); unfortunately, vast quantities of the produced antibodies were directed toward the carrier protein and not the GAS antigen. To avoid such undesired immune responses, the carrier protein may be replaced by universal T-helper (e.g. PADRE), but then an appropriate adjuvant or/and delivery system needs to be incorporated [37].

Although several adjuvants, including experimental (e.g. CFA, cholera toxin, lipid A) and clinical adjuvants (e.g. aluminum salt, MF59 emulsions, monophosphoryl lipid A-based ASO4), are available on the market, they are either considerably toxic, ineffective, or approved only for certain vaccine formulations. They are also expensive. Therefore, there is still a need for new adjuvants that are able induce protective responses against weak antigenic molecules, such as peptides. A variety of such adjuvants and vaccine delivery systems have been investigated. Many of these have proven to be far more effective upon conjugation with an antigen. Such conjugates are often built from a hydrophobic unit and hydrophilic peptide antigen and, therefore, form an amphiphile, which can self-assemble to produce immune stimulatory nanoparticles [12]. Despite the widespread use of these hydrophobic components and nanoparticles in vaccine development, they are not fully defined (in terms of composition, stereochemistry or number of monomers used) [17] and are not completely safe. This places serious limitations on their commercial applications, meaning the demand remains for novel adjuvants for peptide-based subunit vaccines that provide better safety profiles with high immunogenicity, reproducibility, low toxicity, improved stability, biodegradability and biocompatibility.

We recently reported that fully-defined, optically pure natural pHAAs can be conjugated to antigen for self-assembly into nanoparticles and CLANs. In that initial study, of the pHAAs tested (polyvaline, polyphenylalanine, and polyleucine) with different lengths (number of HAA copies), 15 copies of leucine formed the most effective system to induce protective immune responses against GAS infection [25]. In the present study, a variety of pHAAs were conjugated to the peptide antigen (J8 and PADRE) in various arrangements to further optimize the adjuvanting properties of the delivery system. In the first compound mini-series, the pHAA-antigen conjugates were designed to examine the influence of the structural arrangement of the pHAAs and epitopes on the conjugates' (2-4; FIG. 26 b) ability to form nanoparticles and stimulate immune responses.

Mice immunized with Compound 3 were able to produce higher J8-specific systemic IgG titers from primary immunization, compared to mice immunized with the previously studied branched Compound 2 (FIG. 28). The higher antibody titers stimulated by Compound 3 were not related to particle size or morphology, as both branched Compound 2 and linear Compound 3 formed distinct nanoparticles with similar size and evenly distributed CLANs (FIG. 27; FIG. 31; Table 1c). Instead, conjugate efficacy was correlated to the arrangement of the amphiphilic structure, with hydrophilic (J8) and hydrophobic (polyleucine) components situated on both termini of the conjugate. The terminus position of B-cell epitope most likely enables better exposure and presentation to immune cells. The arrangement of epitopes has previously been found to have significant influence on immune responses, despite the similarity of formed particles [26]. However, aggregation behavior also needs to be taken into account, as that of 4 was different to 2 and 3 (FIG. 27).

To further examine pHAA system efficacy, the most promising Compound (4) from the first study was compared with conjugates bearing polymers of a variety of amino acids (hydrophobic amino acids: leucine, glycine, alanine, proline; acidic amino acids: glutamic acid; hydrophilic uncharged amino acid: serine; and a mixture of hydrophobic amino acids: phenylalanine-leucine-alanine; FIG. 26 c), as well as with the experimental ‘gold-standard’ adjuvant (CFA) and clinically tested adjuvants (alum, MF59 and ASO4). Overall, all of the pHAA conjugates (5-11) were able to induce the production of J8-specific antibody titers (FIG. 29), including antigen 1 alone. The ability of 1 to induce antibody production can be explained by its ability (although limited) to self-assemble, as detected by DLS (Table 1c; FIG. 31) but not by TEM (data not shown). Compound 1 was a weak amphiphile, built from highly hydrophilic J8 epitope and relatively hydrophobic PADRE epitope. The poor aggregation ability of 1 resulted in low to no antibody titers (J8- and p145-specific serum IgG and J8-specific saliva IgG; FIG. 29; FIG. 33) and consequently a lack of opsonization capacity from mouse serum (FIG. 29 c). A similar ability to stimulate humoral immunity was seen from Compound 9, which had an identical random coil secondary structure to 1 (FIG. 32 b) and was unable to form prevalent nanoparticles in PBS (FIG. 31; Table 1c; TEM data not shown) as the hydrophilicity of polyglutamic acid formed electrostatic repulsions that prevent structural aggregation at a pH above 4.6 [38]. The inability of polyglutamic acid-antigen conjugate to stimulate significant antibody titers without the presence of additional adjuvant was also reported previously [39].

Interestingly, differences in self-assembly/aggregation potency could not explain the poor immune stimulating efficacy of other compounds when compared with conjugate 5. The conjugation of 15 copies of leucine to 1 (5) has been shown to induce the formation of small nanoparticles that aggregated into CLANs in PBS (FIG. 27). The addition of hydrophobic amino acids alanine (7) and proline (8), hydrophilic uncharged amino acid serine (10) and a mixture of hydrophobic amino acids (phenylalanine-leucine-alanine; 11) showed a similar nanoparticle pattern to 5. The ability of polyserine (despite its hydrophilicity) to trigger aggregation once conjugated to other peptides was previously reported [40]. In contrast, polyglycine-conjugate 6 formed slightly larger nanoparticles that aggregated into less-distinctive CLANs and long rod nanoparticles. It was reported that polyglycine (more than 9 copies) has the tendency to form elongated rods [41]. Nevertheless, despite the similarities and differences portrayed, conjugates 6-11 still did not stimulate higher antibody titers than antigen 1 (FIG. 29 a).

Good and co-workers showed that even minor modification of a peptide epitope can greatly influence conformation and immunogenicity [42]. As J8 is expected to mimic the helical conformation of M protein, we examined the influence of pHAAs on the conformation of the conjugates. Polyleucine (5) induced the highest extent of α-helicity among all compounds (FIG. 32 a). As expected, polyglycine did not affect the conformation of antigen 1 (6; FIG. 32 b). But surprisingly, neither did alanine (FIG. 32 a), despite it having been previously shown that 16-residue alanine-based peptides have a stable α-helix formation due to the high helix-forming potential of alanine [43, 44]. Instead, conjugate 7 showed a mixture of α-helix and β-sheets, matching other studies that showed that polyalanine-based peptides form conformational conversions from α-helical to β-pleated-sheet structures [45, 46]. On the other hand, the polyproline conjugate (8) adopted a random coil (FIG. 32 a), rather than polyproline II conformation (as per lack of the characteristic absorption minimum around 230 nm) [47]. Likewise, Compounds 9-11 predominantly adopted random coil conformations (FIG. 32); suggesting that particle formation and conformation both play crucial roles in pHAA adjuvanting capacity. Ultimately, poyleucine was found to be the most effective among the tested poly(amino acid) sequences. To further examine the efficacy of this system, conjugate 5 was compared with commercial adjuvants.

Adjuvanted Compound 1 and polyleucine-conjugate 5 were able to induce antibody responses against both J8 and p145 (FIG. 29 a and FIG. 33, respectively). Compound 5 induced a significant level of antibody titers in mice following the first immunization, compared to PBS, as did all commercial adjuvants. Following the second boost, the IgG titers stimulated by 5 were equivalent to those induced by ‘gold-standard’ adjuvant CFA and the best performing human-grade adjuvant MF59. However, it is worth noting that one mouse in the CFA-immunized group died due to adjuvant toxicity. Swelling, necrosis at the injection site, and reduction of mice physical activity following CFA immunization were also observed. In contrast, no any adverse effects were detected in other immunized groups. When excluding toxic CFA, the pHAA system was at least as effective as the commercial adjuvants in inducing antibody production in mice following single boost immunization.

Among all tested compounds and adjuvants, only polyleucine conjugate 5, CFA- and MF59-adjuvanted antigen were able to induce the production of mucosal IgG in saliva (FIG. 29 b). Importantly, only antibodies present in mice sera immunized with 1+CFA, 1+MF59 and 5 were of high enough quantity and quality to kill GAS bacteria in the opsonization experiment (FIG. 29 c). Interestingly, despite the relatively high antibody titers generated by 1+alum and 1+AS04, these antibodies were not opsonic against GAS, further demonstrating the capability of polyleucine as an adjuvant. It has been shown previously that high antibody titers do not necessarily correlate to high opsonization capability [48]. In contrast, we demonstrated that high opsonic ability of sera correlates directly with efficient bacterial clearance in mice challenged with GAS [25]. Notably, conjugate 5 did not induce production of antibodies against polyleucine (FIG. 34). Thus, the polyleucine-based self-adjuvanting nanoparticle system was determined to be the prime vaccine delivery strategy compared to all tested poly(amino acids) and commercial adjuvants, with exception of the expensive adjuvant MF59, which showed similar efficacy.

Conclusion

This study reported on the synthesis, characterization, and immunological evaluation of pHAA-based vaccine candidates against GAS. Epitope arrangement within the pHAA conjugate was determined to be the most influential factor in the generation of an effective immune response, rather than particle morphology. Linear vaccine constructs with hydrophobic polyleucine and hydrophilic J8 separated by amphiphilic PADRE epitope induced the production of J8-specific antibodies in mice more effectively than other arrangements. Among the tested pHAAs, a helical antigen conjugate harboring 15 copies of leucine evoked the strongest immune responses (highest antibody titers and opsonic activity); these were comparable to commercial adjuvants, including CFA. These findings demonstrate the significant capacity of polyleucine as a self-adjuvanting peptide component for vaccine design.

### Example 6

Introduction

COVID-19 pandemic caused by SARS-CoV-2 infection has caused major global health and economic disasters since its outbreak in late 2019 [1, 2]. Hundreds of vaccine candidates have been produced, with more than 50 of them in clinical trials by the end of 2020 [3]. However, while some of them appear to be efficacious, most may have serious sides effects and require extreme temperature transport and storage conditions [4-6].

The COVID-19 infections occur through inhalation of contaminated aerosol arising from exhalation by an infected patient [7]. Following inhalation or other mucosal contacts, SARS-CoV-2 binds via its spike protein (SARS-2-S) to angiotensin converting enzyme-2 (ACE2) expressed by human cells, including lung pneumocytes type-II, cardiomyocytes, enterocytes, kidney cells, and macrophages. The binding allows SARS-CoV-2 to fuse with host cell [8]. Then, viral RNA is translated into polyprotein precursor that is further cleaved to yield functional structural and non-structural proteins allowing replication of the virus [9]. In SARS-CoV and SARS-CoV-2 convalescent patients' sera, 90% of antibodies are generated against spike protein and 90% of the “neutralizing” antibodies are directed against receptor binding domain (RBD) of spike protein [10-13]. These humoral immune responses were found to be crucial to develop protection against infection. Unfortunately, the use of vaccines that include or encode SARS-2-S or RBD is associated with the risk of immunopathological pro-inflammatory responses, similar to those occurring after natural infection [14-17]. Moreover, some of the anti-S-protein antibodies can enhance virus entry into host cells, promoting infection instead of protecting against it [18, 19].

To avoid the disadvantages of virus-, protein- and RNA-based vaccines, minimal epitopes derived from RBD can be selected for vaccine design. The neutralizing epitope-based vaccine generates antibodies that prevent viral entry into the human cells without causing any undesired immune reactions therefore retaining efficacy without compromising safety. However, such peptide-based vaccine needs appropriate delivery system and/or adjuvant [20-22].

The sequences of five potential SARS-CoV-2 epitopes, designed based on the known SARS-CoV spike protein epitopes [12, 19, 23-26] as follows:

(S623-639, B1; SEQ ID NO: 15)
AIHADQLTPTWRVYSTG;
(S469-508, B2; SEQ ID NO: 16)
STEIYQAGSTPCNGVEGFNCYFPLQSYGFQPTNGVGYQPY;
(S445-483, B3; SEQ ID NO: 17)
VGGNYNYLYRLFRKSNLKPFERDISTEIYQAGSTPCNGV;
(S559-572, B4; SEQ ID NO: 18)
FLPFQQFGRDIADT;
and
(S366-395, B5; SEQ ID NO: 19)
SVLYNSASFSTFKCYGVSPTKLNDLCFTNV.

Firstly, the epitopes' ability to induce antibody production was examined upon co-administration with complete Freund's adjuvant (CFA) in BALB/c mice. Then, the most effective epitope was modified with poly-leucine moiety and formulated into liposomes. The formulation was examined toward anti-RBD antibody production in C57BL/6 mice. The produced antibodies were tested for their ability to inhibit binding between RBD and ACE2 in vitro.

### Experimental

### Synthesis and Purification of B1-B5

Peptides B1-B5 were synthesized by microwave-assisted standard Fmoc-solid-phase peptide synthesis using SPS model CEM Discovery reactor [27]. Briefly, peptides were synthesised on rink amide MBHA (substitution ratio: 0.51 mmol/g, 196 mg, 0.1 mmol scale); the resin was swelled overnight in N′N′-dimethylformamide (DMF). The Fmoc group was removed using 20% piperidine/DMF for 2 and 5 min at 70° C. Amino acids were activated with 0.5 M 1-[bis(dimethylamino)methylene]-1H-1,2,3-triazolo[4,5-b] pyridinium 3- oxid hexafluorophosphate (HATU) (4 equiv, 0.8 mL) and N,N-diisopropylethylamine (DIPEA) (5.2 equiv, 91 μL) in DMF and added for 5 and 10 min coupling. Unreacted resin was acetylated with acetic anhydride, DIPEA and DMF (5:5:90) at 70° C., twice (5 and 10 min). The Fmoc deprotections, amino acids activation and coupling to the resin were repeated until the desired peptides were achieved. The finished peptides were cleaved from the resin using 95% trifluoroacetic acid (TFA), 2.5% trisopropylsilane and 2.5% water. The cleavage cocktail was removed using vacuum, before the peptides were washed, dissolved, filtered, and freeze dried. These peptides were dissolved in solvent B (acetonitrile/water (50:50)) prior to lyophilization. The products were purified by RP-HPLC using a C18 Vydac column with solvent gradient of 45-65% solvent B over 30 min. Analytical analysis was performed using a Shimadzu instrument (C18 column).

B1 peptide (623AIHADQLTPTWRVYSTG639) at S1/S2 cleavage/priming site, Yield: 75%. Molecular weight: 1957.18. ESI-MS, [M+2H]2+ m/z 979.6 (calc. 979.5), [M+2H]2+ m/z 653.5 (calc. 653.39). t_{R}=17.97 min (0 to 100% solvent B; C18 column); purity ≥99%.

B2 peptide (469STEIYQAGSTPCNGVEGFNCYFPLQSYGFQPTNGVGYQPY508) at receptor binding motif of RBD, Yield: 45%. Molecular weight: 4425.8. ESI-MS [M+3H]3+ m/z 1476.8 (calc. 1476.27), [M+4H]4+ m/z 1108.0 (calc. 1107.45). t_{R}=21.15 min (0 to 100% solvent B; C18 column); purity ≥99%.

B3 peptide (444GVGGNYNYLYRLFRKSNLKPFERDISTEIYQAGSTPCNGV483) at receptor binding motif of RBD. Yield: 50%. Molecular weight: 4593.2. ESI-MS, [M+3H]3+m/z 1532.3 (calc. 1532.07), [M+4H]4+ m/z 1149.1 (calc. 1149.3), [M+5H]5+ m/z 919.5 (calc. 919.64). t_{R}=21.49 min (0 to 100% solvent B; C18 column); purity ≥99%.

B4 peptide (559FLPFQQFGRDIADT572) near S1/S2 cleavage/priming site. Yield: 80%. Molecular weight: 1675.8 EST-MS, [M+1H]1+ m/z 1676.8 (calc. 1676.8), [M+2H]2+ m/z 839.4 (calc. 839.9), [M+3H]3+ m/z 559.9 (calc. 559.6). t_{R}=19.35 min (0 to 100% solvent B; C18 column); purity ≥99%.

B5 peptide (366SVLYNSASFSTFKCYGVSPTKLNDLCFTNV395) at NTD of receptor binding domain but not in direct contact with ACE2. Yield: 55%. Molecular weight: 3348.6. ESI-MS, [M+2H]2+ m/z 1675.2 (calc. 1675.3), [M+3H]3+ m/z 1117.1 (calc. 1117.2), [M+4H]4+m/z 838.6 (calc. 838.15). t_{R}=17.2 min (0 to 100% solvent B; C18 column); purity ≥99%.

### Synthesis and Purification of (Leu)₁₀-B3 and (Leu)₁₀-PADRE

(Leu)₁₀-B3 was synthesized analogously as B3 above, except ten leucines moieties were coupled to its N-terminus following by final acetylation.

(Leu)₁₀-B3: (LLLLLLLLLL-GVGGNYNYLYRLFRKSNLKPFERDISTEIYQAGSTPCNGV). Yield: 40%. Molecular weight: 5747.40. ESI-MS, [M+4H]⁴⁺ m/z 1437.2 (calc. 1437.85), [M+5H]⁵⁺ m/z 1149.8 (calc. 1150.48), [M+6H]⁶⁺ m/z 958.9 (calc. 958.90). t_{R}=25.64 min (0 to 100% solvent B; C4 column); purity ≥99%.

(Leu)₁₀-PADRE was synthesized analogously as (Leu)₁₀-B3 above, except B3 was replaced with universal T-helper epitope PADRE (AKFVAAWTLKAAA; SEQ ID NO:7).

(Leu)₁₀-PADRE: Yield 45%. Molecular weight: 2521.27. ESI-MS, [M+2H]²⁺ m/z 1260.6 (calc. 1261.64), [M+3H]³⁺ m/z 841.0 (calc. 841.42). t_{R}=37.10 min (0 to 100% solvent B; C4 column); purity ≥99%.

Liposomal Formulation L1

Dipalmitoylphosphatidylcholine (DPPC), didodecyldimethylammonium bromide (DDAB), and cholesterol were each dissolved in 1 mL of chloroform to achieve final concentrations of 10 mg/mL, 10 mg/mL and 5 mg/mL, respectively. DPPC (0.5 mL), DDAB (0.2 mL) and cholesterol (0.2 mL) were added to a round bottom flask containing 2 mL of chloroform. (Leu)₁₀-PADRE (0.6 mg) and (Leu)₁₀-B3 (0.6 mg) were dissolved in 1 mL methanol and added to the flask. The solvents were then slowly evaporated under reduced pressure using a rotatory evaporator. All residual solvent was removed under vacuum overnight. Lipid film was rehydrated gradually with 1 mL of PBS at 56° C. to produce 0.6 mg/mL concentration of (Leu)₁₀-PADRE and 0.6 mg/mL concentration of (Leu)₁₀-B3 in L1. The liposome was then ultrasonicated (10 min, 40% power, 50% pulse) 3 times. Size distribution by number˜100 nm; PDI=0.38; charge=15 mV.

Liposomal Formulation L2

L2 was prepared in an identical manner to L1 except 1.2 mg of (Leu)₁₀-B3 instead of 0.6 mg was dissolved in 1 mL methanol. Thus, producing the final 0.6 mg/mL concentration of (Leu)₁₀-PADRE and 1.2 mg/mL concentration of (Leu)₁₀-B3 in L2. Size distribution by number ˜100 nm; PDI=0.54; charge=16 mV.

Subcutaneous Immunisation in Mice (Experiment 1)

Six-week-old female BALB/c mice were purchased from the University of Queensland's Biological Resources (UQBR) (QLD, Australia). Mice were housed in the Australian Institute for Bioengineering and Nanotechnology (AIBN) Animal Facility. The mice were acclimatized for 7 days before any experiment was conducted. All mice (5 per group) were immunized subcutaneously in tail once. Negative control mice received 50 μL of PBS, while positive control mice received RBD (30 μg) dissolved in 25 μL of PBS and emulsified with 25 μL of CFA. The vaccine candidates' groups were given Compounds B1, B2, B3, B4, or B5 (30 μg) dissolved in PBS (25 μL) and emulsified with 25 μL CFA.

Collection of Serum (Experiment 1)

Blood was collected via heart puncture (1 mL) at day 14. The collected blood samples were centrifuged at 3,600 rpm for 10 min. Serum was collected from the supernatant and was kept at −80° C. until further analysis.

Subcutaneous Immunisation in Mice (Experiment 2)

Six-week-old female C57BL/6 mice were purchased from the UQBR (QLD, Australia). Mice were housed in the AIBN Animal Facility. The mice were acclimatized for 7 days before any experiment was conducted. All mice (5 per group) were immunized subcutaneously in tail three times at day 0, 14 and 28. Negative control mice received 50 μL of PBS, while positive controls mice received RBD (30 μg) dissolved in PBS (25 μL) and emulsified with 25 μL CFA. Three other mice groups were immunized with B3 (30 μg) dissolved in PBS (25 μL) and emulsified with 25 μL M1F59; L1 (50 μL) and L2 (50 μL), respectively.

Collection of Serum (Experiment 2)

Blood was collected via heart puncture (1 mL) at day 41. The collected blood samples were centrifuged at 3,600 rpm for 10 min. Serum was collected from the supernatant and was kept at −80° C. until further analysis.

Antibody Titer Detection by ELISA

Antigen-specific antibody IgG titres were detected using an ELISA. Polystyrene high-affinity plates were coated with B1, B2, B3, B3, B4, B5, or RBD (pH 9.6, 50 μg in 10 mL of carbonate coating buffer) with amount of 100 μL per well, and incubated at 37° C. for 90 min. Then, the plates' content was discarded and 150 μL of 5% skim milk (dissolve with PBS-Tween 20 buffer) was added in each well. After overnight incubation at 4° C., the plates were washed with distilled water (4×) and PBS-Tween 20 buffer (3×). A 2 μL of undiluted sera sample was added to the first row of the plate containing 198 μL of 0.5% skim milk in PBS-Tween 20 buffer (1:100 dilution). All plates were incubated for 90 min in a 37° C. incubator. The plates were washed before adding 100 μL per well of 1:3000 diluted peroxidase-conjugated goat anti-mouse IgG (in 0.5% skim milk). The plates were incubated for 90 min at 37° C. and then washed. A 100 μL amount of o-phenylenediamine dihydrochloride (OPD) substrate was added in each well. A 50 μL amount of stop solution (1N H₂SO₄) was added per well after the plates were incubated for 20 min in a dark environment at room temperature. The absorbance (OD) of stained RBD-specific IgG was measured at 450 nm.

Competitive ELISA Test

Serum samples were tested for anti-RBD neutralizing antibodies through enzyme-linked immunosorbent assays (ELISA), in a similar approach to reported methods with some modifications [28, 29]. First, 96-well high affinity binding polystyrene plates were coated with carbonate coating buffer containing 25 μg of RBD per plate for first experiment and 5 μg for second, including two extra plates for negative control group (PBS immunized group) and ACE2 calibration curve. RBD placed in well-plates were allowed to coat the wells for 90 min at 37° C. Plates were emptied and blocked by coating the wells with 2% bovine serum albumin (BSA) to block remaining high affinity sites within the wells, binding was left overnight to bind at 4° C. The plates were emptied and washed with distilled water (5×) and PBS-Tween 20 buffer (4×). A 20 uL amount of neat serum samples were added to sera plates (including PBS group) and serum were diluted in 0.5% BSA, starting from 1:4 and 1:10 dilution for experiments 1 and 2, respectively. No serums were added to calibration curve plate (only 0.5% BSA). The antibodies (available in the serum) placed in well-plates were allowed to bind to RBD for 90 min at 37° C. The plates were emptied and washed (as previous). A 20 μg amount of human angiotensin converting enzyme conjugated to human IgG-Fc (ACE2-Fc) (1 mg dissolved in 1 mL purified water) was mixed with 10 mL of 0.5% BSA, then 100 μL of the mixture were added to each well (except for calibration curve plate). In the calibration curve plate, ACE2-Fc was serially diluted from 2 ng/μL (200 ng/100 μL/well) further down to 0.1 ng/μL (10 ng/100 μL/well). The ACE2-Fc placed in well-plates were allowed to bind to for 90 min at 37° C. The plates were emptied and washed (as previously). 100 μL of diluted goat anti-human IgG-Fc-HRP secondary antibodies (3.33 μL of antibodies in 10 mL of 0.5% BSA) were added in each well for all the plates. These secondary antibodies were allowed to bind to ACE2-Fc for 90 min at 37° C. The plates were then emptied and washed (as previous). The OPD substrate was prepared and 100 μL were added per each well. The OPD solution was allowed to react for 20 min at room temperature in a dark environment, before the reaction was stopped by adding 50 μL of 1N H₂SO₄ solution to each well. Absorbance at 450 nm was measured using a Spectra Max Microplate reader. In order to determine the content of bound and unbound ACE2 in each well, the calibration curve (n=4 for first experiment and n=8 for second experiment) was fitted to logistic function (R²>0.98), Equation 1. The best fit equation was used with absorbance (y) values of each well to determine bound ACE2 content (x) by interpolation. Percentage of inhibition is determined from percent of unbound amount of ACE2, Equation 2.

$$y = \frac{{A}_{1} - {A}_{2}}{1 + {( x / {x}_{o} )}^{p}} + {A}_{2} \qquad Equation 1$$

$$Inhibition \% = \frac{ACE {2}_{Total} - ACE {2}_{Bound}}{A C E {2}_{Total}} \times 100 \qquad Equation 2$$

Where y is Absorbance readings, A₁ and A₂ are minimum and maximum absorbance readings, respectively, x is amount of bound ACE2 concentration in ng/100 μL/well to be used in equation 2, and xo is x-axis (ACE2 concentration) centre point, p is power exponent describes rate of change in absorbance signal with changing amount of bound ACE2, and ACE2 total is 200 ng/100 μL/well.

The validity of the method was tested for suitability, bias and accuracy, specificity, sensitivity and selectivity, by constructing a well-fitted calibration curve, adding irrelevant sera to varying known ACE2 concentrations as well as by adding secondary antibodies to different test sera in absence of ACE2. The calibration curve absorbance readings remained the same in presence of irrelevant animal sera in the range of 10 to 200 ng/100 μL/well, i.e. corresponding to 0-95% range of inhibition, and absorbance readings were non-significant (p<0.05, n=4) in absence of ACE2 as well as at very low ACE2 concentrations within the calibration curve <10 ng/100 μL/well (p<0.05, n=4). The ACE2 concentrations were adjusted to achieve saturation of RBD amount coated on the plate.

Results and Discussion

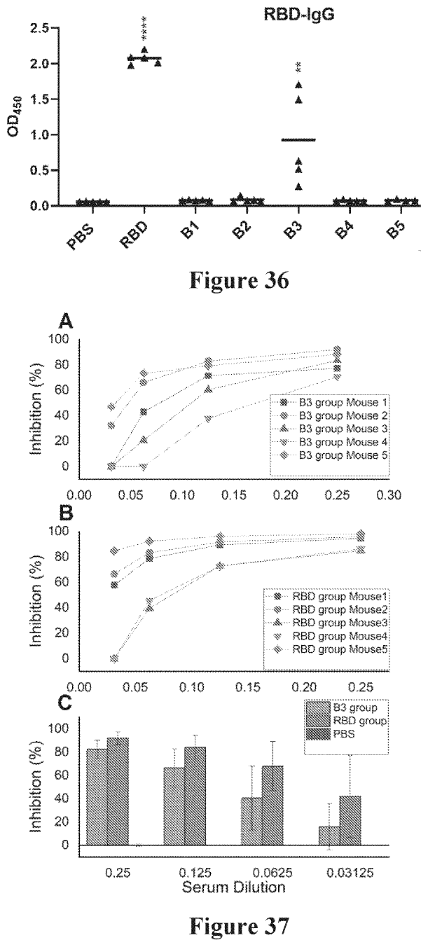

Five potential SARS-CoV-2 epitopes were designed and synthesized (B1-B5), taking into consideration known SARS-CoV epitopes and ACE2-RBD critical binding amino acid residues. Epitopes were co-administered with “gold standard” experimental adjuvant (CFA) in BALB/c mice. The mice were examined toward production of antibodies against RBD region of SARS-CoV-2 spike protein (FIG. 36). As expected RBD adjuvanted with CFA generated high antibody titers against itself. Epitope (B3) was able to trigger statistically significant antibody production against RBD when co-administered with CFA. Mice immunized with B1 did not generate anti-RBD IgG as expected since the sequence of B1 is located out of spike protein RBD domain. Moreover, B1 also did not generate significant antibody titers against itself when examined by ELISA.

Despite differences in antibody responses between RBD and B3 groups, the ability of sera to inhibit interaction of viral RBD with ACE2 were almost identical (FIG. 37). Sera from all BALB/c mice (both B3 and RBD groups) were able to completely inhibit RBD binding to ACE2 at high sera concentration. Upon 32-fold dilution RBD sera from 3/5 mice maintained inhibition potential, while B3 sera from 2/5 mice had still ability to block RBD binding. Consequently, B3 was selected as promising epitope for further studies.

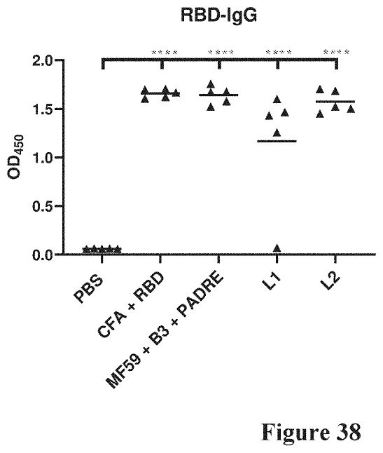

We have demonstrated previously that a poly-leucine delivery system was the most efficient among tested poly(hydrophobic amino acid) systems to generate high antibody titers. Therefore, B3 was conjugated to a poly-leucine unit, forming (Leu)₁₀-B3. In addition, universal human T-helper PADRE (AKFVAAWTLKAAA; SEQ ID NO:7) was also conjugated to the polyleucine unit, producing (Leu)₁₀-PADRE. PADRE is commonly used to enhance the quality and longevity of humoral immune responses [30]. Both conjugates were anchored (via polyleucine moiety) to liposomes to further enhance vaccine potency. Mice (C57BL/6) were immunized subcutaneously with 50 μL of L1 (bearing 30 μg of (Leu)₁₀-PADRE and 30 μg of (Leu)₁₀-B3) and L2 (bearing 30 μg (Leu)₁₀-PADRE and 60 μg (Leu)₁₀-B3) (FIG. 38). As positive control RBD adjuvanted with CFA and B3+PADRE adjuvanted with MF59 were used. Both vaccine candidates generated high antibody titters against RBD, especially L2 generated same level of RBD-IgG as positive controls for all mice.

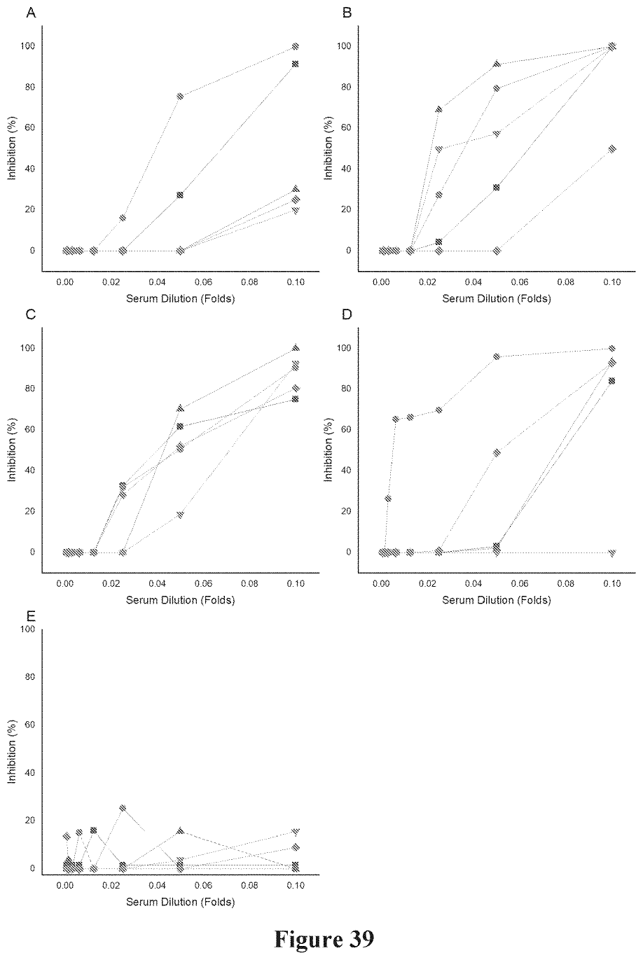

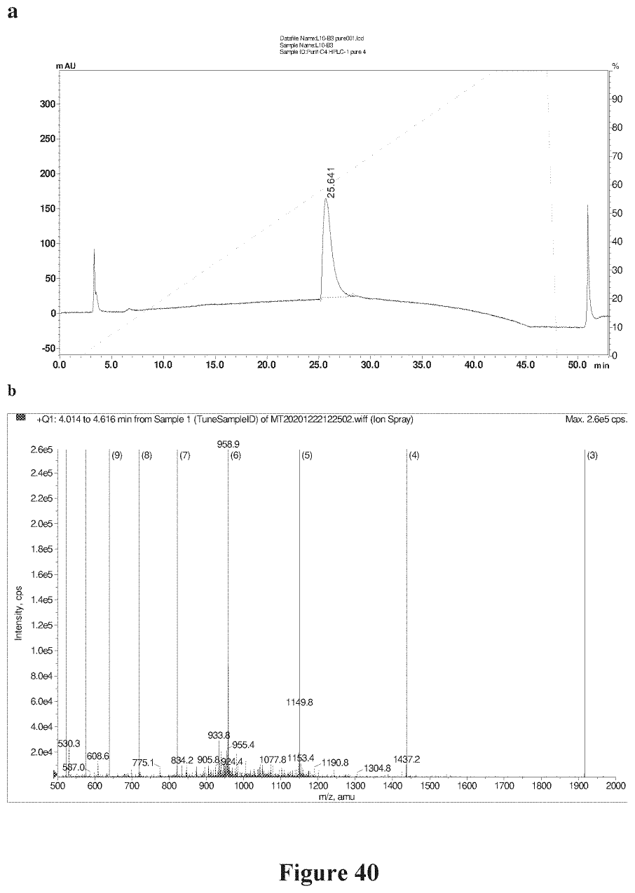

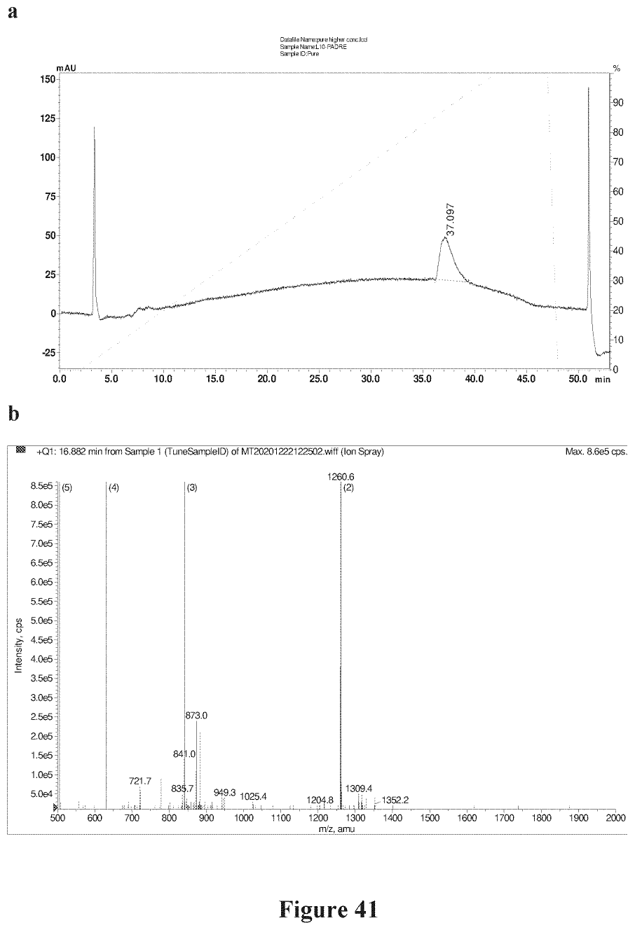

The ability of sera to inhibit interaction of viral RBD with ACE2 were tested for all the groups: PBS, CFA+RBD, B3+PADRE+MF59, L1 and L2 (FIG. 39); however, higher sera dilution were used than in the first experiment (FIG. 37). In the similar dilution, CFA+RBD immunized C57BL/6 mice sera appears to be less effective than sera from BALB/c mice immunized with the same adjuvanted protein fragment (CFA+RBD). All other groups at highest sera concentration inhibited RBD/ACE2 binding efficiently for almost all mice sera. However, at dilution 1/20 this inhibition was reduced, to fall-down practically to zero at 1/100 dilution. Interestingly, anti-RBD IgG titers were measured at 1/100 dilution and showed high OD values, suggesting that large number of produced antibodies had no inhibitory activity towards RBD/ACE2 binding, even in positive control group (RBD+CFA). Therefore, further improvement of the delivery system is required, which may include: a) conjugation of PADRE to B3 (T-helper conjugation to B-cell epitope is often recommend for vaccine design); b) examination of conformational properties of B3 and its modification by replacement of polyleucine with other hydrophobic amino acid (e.g. beta sheet inducing polyvaline sequence); c) optimization of liposomal formulation; and d) replacement of PADRE T-helper with carrier protein.

Conclusion

We have examined several peptide fragments of SARS-CoV-2 spike protein as potential peptide antigens. Among them only B3 generated high IgG titers against RBD region of spike protein when administered with CFA. Moreover, these antibodies were able to inhibit RBD/ACE2 binding. Once administered as polyleucine conjugate ((Leu)₁₀-B3) in liposomes the antigen was also able to generate high IgG titers which were able to inhibit RBD/ACE2 binding at high concentration.

### REFERENCES—EXAMPLE 1

1. Skwarczynski M, Toth I. Peptide-based synthetic vaccines. Chem Sci 7, 842-854 (2016).
2. Purcell A W, McCluskey J, Rossjohn J. More than one reason to rethink the use of peptides in vaccine design. Nat Rev Drug Discovery 6, 404-414 (2007).
3. Steer A C, et al. Status of research and development of vaccines for Streptococcus pyogenes. Vaccine 34, 2953-2958 (2016).
4. Carapetis J R, Steer A C, Mulholland E K, Weber M. The global burden of group A streptococcal diseases. Lancet Infect Dis 5, 685-694 (2005).
5. Paar J A, et al. Prevalence of Rheumatic Heart Disease in Children and Young Adults in Nicaragua. Am J Cardiol 105, 1809-1814 (2010).
6. Gorton D, Govan B, Olive C, Ketheesan N. B- and T-Cell Responses in Group A Streptococcus M-Protein- or Peptide-Induced Experimental Carditis. Infect Immun 77, 2177-2183 (2009).
7. Brandt E R, et al. New multi-determinant strategy for a group A streptococcal vaccine designed for the Australian Aboriginal population. Nat Med 6, 455-459 (2000).
8. Azmi F, Ahmad Fuaad A A, Skwarczynski M, Toth I. Recent progress in adjuvant discovery for peptide-based subunit vaccines. Hum Vaccin Immunother 10, 778-796 (2014).
9. Petrovsky N, Aguilar J C. Vaccine adjuvants: Current state and future trends. Immunol Cell Biol 82, 488-496 (2004).
10. Montomoli E, Piccirella S, Khadang B, Mennitto E, Camerini R, De Rosa A. Current adjuvants and new perspectives in vaccine formulation. Expert Rev Vaccines 10, 1053-1061 (2011).
11. Reed S G, Orr M T, Fox C B. Key roles of adjuvants in modern vaccines. Nat Med 19, 1597-1608 (2013).
12. Skwarczynski M, et al. Polyacrylate dendrimer nanoparticles: a self-adjuvanting vaccine delivery system. Angew Chem Int Ed Engl 49, 5742-5745 (2010).
13. Ahmad Fuaad A A, et al. Polymer-peptide hybrids as a highly immunogenic single-dose nanovaccine. Nanomedicine (Lond) 9, 35-43 (2014).
14. Liu T Y, et al. Self-adjuvanting polymer-peptide conjugates as therapeutic vaccine candidates against cervical cancer. Biomacromolecules 14, 2798-2806 (2013).
15. Chandrudu S, et al. Linear and branched polyacrylates as a delivery platform for peptide-based vaccines. Ther Deliv 7, 601-609 (2016).
16. Liu T Y, et al. Polyacrylate-based delivery system for self-adjuvanting anticancer peptide vaccine. J Med Chem 58, 888-896 (2015).
17. Skwarczynski M, Toth I. Lipid-core-peptide system for self-adjuvanting synthetic vaccine delivery. In: Bioconjugation Protocols (ed\hat{ }(eds Mark S S). Humana Press (2011).
18. Sohma Y, et al. O-N intramolecular acyl migration reaction in the development of prodrugs and the synthesis of difficult sequence-containing bioactive peptides. Biopolymers 76, 344-356 (2004).
19. Georgousakis M M, Hofmann A, Batzloff M R, McMillan D J, Sriprakash K S. Structural optimisation of a conformational epitope improves antigenicity when expressed as a recombinant fusion protein. Vaccine 27, 6799-6806 (2009).
20. Trent A, et al. Peptide Amphiphile Micelles Self-Adjuvant Group A Streptococcal Vaccination. AAPS J 17, 380-388 (2015).
21. Malkov S N, Zivkovic M V, Beljanski M V, Hall M B, Zaric S D. A reexamination of the propensities of amino acids towards a particular secondary structure: classification of amino acids based on their chemical structure. Journal of Molecular Modeling 14, 769-775 (2008).
22. Bouchard M, Zurdo J, Nettleton E J, Dobson C M, Robinson C V. Formation of insulin amyloid fibrils followed by FTIR simultaneously with CD and electron microscopy. Protein Science 9, 1960-1967 (2000).
23. Yang C-A, et al. Correlation of copper interaction, copper-driven aggregation, and copper-driven H2O2 formation with Aβ40 conformation. International Journal of Alzheimer's Disease 2011, (2010).
24. Park H S, Cleary P P. Active and passive intranasal immunizations with streptococcal surface protein C5a peptidase prevent infection of murine nasal mucosa-associated lymphoid tissue, a functional homologue of human tonsils. Infect Immun 73, 7878-7886 (2005).
25. Skwarczynski M, Toth I. Recent advances in peptide-based subunit nanovaccines. Nanomedicine (London, U K) 9, 2657-2669 (2014).
26. Zhao G, Chandrudu S, Skwarczynski M, Toth I. The application of self-assembled nanostructures in peptide-based subunit vaccine development. Eur Polym J 93, 670-681 (2017).
27. Negahdaripour M, Golkar N, Hajighahramani N, Kianpour S, Nezafat N, Ghasemi Y. Harnessing self-assembled peptide nanoparticles in epitope vaccine design. Biotechnol Adv 35, 575-596 (2017).
28. Liao J, et al. Linear aggregation of gold nanoparticles in ethanol. Colloids and Surfaces A: Physicochemical and Engineering Aspects 223, 177-183 (2003).
29. Friedlander S K, Ogawa K, Ullmann M. Elasticity of nanoparticle chain aggregates: implications for polymer fillers and surface coatings. Powder Technol 118, 90-96 (2001).
30. Cella M, Engering A, Pinet V, Pieters J, Lanzavecchia A. Inflammatory stimuli induce accumulation of MHC class II complexes on dendritic cells. Nature 388, 782-787 (1997).
31. Ma D Y, Clark E A. The role of CD40 and CD154/CD40L in dendritic cells. Semin Immunol 21, 265-272 (2009).
32. Moyano D F, et al. Nanoparticle Hydrophobicity Dictates Immune Response. J Am Chem Soc 134, 3965-3967 (2012).
33. Rad-Malekshahi M, et al. Self-Assembling Peptide Epitopes as Novel Platform for Anticancer Vaccination. Mol Pharm 14, 1482-1493 (2017).

### REFERENCES—EXAMPLE 2

[1] F. Bray, J. Ferlay, I. Soerjomataram, R. L. Siegel, L. A. Torre, A. Jemal, Global cancer statistics 2018: GLOBOCAN estimates of incidence and mortality worldwide for 36 cancers in 185 countries, CA: a cancer journal for clinicians 68(6) (2018) 394-424.
[2] D. Maine, S. Hurlburt, D. Greeson, Cervical cancer prevention in the 21st century: cost is not the only issue, American journal of public health 101(9) (2011) 1549-1555.
[3] T. Y. Liu, W. M. Hussein, I. Toth, M. Skwarczynski, Advances in peptide-based human papillomavirus therapeutic vaccines, Current topics in medicinal chemistry 12(14) (2012) 1581-92.
[4] T.-Y. Liu, W. M. Hussein, Z. Jia, Z. M. Ziora, N. A. J. McMillan, M. J. Monteiro, I. Toth, M. Skwarczynski, Self-Adjuvanting Polymer-Peptide Conjugates As Therapeutic Vaccine Candidates against Cervical Cancer, Biomacromolecules 14(8) (2013) 2798-2806.
[5] S. G. Reed, S. Bertholet, R. N. Coler, M. Friede, New horizons in adjuvants for vaccine development, Trends in Immunology 30(1) (2009) 23-32.
[6] R. J. Nevagi, I. Toth, M. Skwarczynski, 12-Peptide-based vaccines, in: S. Koutsopoulos (Ed.), Peptide Applications in Biomedicine, Biotechnology and Bioengineering, Woodhead Publishing 2018, pp. 327-358.
[7] T. Y. Liu, A. K. Giddam, W. M. Hussein, Z. Jia, N. A. McMillan, M. J. Monteiro, I. Toth, M. Skwarczynski, Self-adjuvanting therapeutic peptide-based vaccine induce CD8+ cytotoxic T lymphocyte responses in a murine human papillomavirus tumor model, Current drug delivery 12(1) (2015) 3-8.
[8] T.-Y. Liu, W. M. Hussein, A. K. Giddam, Z. Jia, J. M. Reiman, M. Zaman, N. A. J. McMillan, M. F. Good, M. J. Monteiro, I. Toth, M. Skwarczynski, Polyacrylate-Based Delivery System for Self-adjuvanting Anticancer Peptide Vaccine, Journal of Medicinal Chemistry 58(2) (2015) 888-896.
[9] W. M. Hussein, T. Y. Liu, Z. Jia, N. A. J. McMillan, M. J. Monteiro, I. Toth, M. Skwarczynski, Multiantigenic peptide-polymer conjugates as therapeutic vaccines against cervical cancer, Bioorganic & medicinal chemistry 24(18) (2016) 4372-4380.
[10] M. Khongkow, T.-Y. Liu, S. Bartlett, W. M. Hussein, R. Nevagi, Z. Jia, M. J. Monteiro, J. W. Wells, U. Rungsardthong Ruktanonchai, M. Skwarczynski, I. Toth, Liposomal formulation of polyacrylate-peptide conjugate as a new vaccine candidate against cervical cancer, Precision Nanomedicine 1(3) (2018) 183-193.
[11] Y. Guo, C. Sakonsinsiri, I. Nehlmeier, M. A. Fascione, H. Zhang, W. Wang, S. Pöhlmann, W. B. Turnbull, D. Zhou, Compact, Polyvalent Mannose Quantum Dots as Sensitive, Ratiometric FRET Probes for Multivalent Protein-Ligand Interactions, Angewandte Chemie (International ed. in English) 55(15) (2016) 4738-4742.

### REFERENCES—EXAMPLE 3

1. Purcell, A. W.; McCluskey, J.; Rossjohn, J. More than one reason to rethink the use of peptides in vaccine design. Nat. Rev. Drug Discovery 2007, 6, 404-414.
2. Skwarczynski, M.; Toth, I. Peptide-based synthetic vaccines. Chemical Science 2016, 7, 842-854.
3. Nevagi, R. J.; Toth, I.; Skwarczynski, M. 12-Peptide-based vaccines. In Peptide Applications in Biomedicine, Biotechnology and Bioengineering, Koutsopoulos, S., Ed. Woodhead Publishing: 2018; pp 327-358.
4. Giddam, A. K.; Zaman, M.; Skwarczynski, M.; Toth, I. Liposome-based delivery system for vaccine candidates: constructing an effective formulation. Nanomedicine 2012, 7, 1877-1893.
5. Azizi, A.; Kumar, A.; Diaz-Mitoma, F.; Mestecky, J. Enhancing Oral Vaccine Potency by Targeting Intestinal M Cells. PLOS Pathogens 2010, 6, e1001147.
6. Marasini, N.; Skwarczynski, M.; Toth, I. Oral delivery of nanoparticle-based vaccines. Expert Rev. Vaccines 2014, 13, 1361-76.
7. Loukas, A.; Hotez, P. J.; Diemert, D.; Yazdanbakhsh, M.; McCarthy, J. S.; Correa-Oliveira, R.; Croese, J.; Bethony, J. M. Hookworm infection. Nat Rev Dis Primers 2016, 2, 16088.
8. Bethony, J.; Chen, J.; Lin, S.; Xiao, S.; Zhan, B.; Li, S.; Xue, H.; Xing, F.; Humphries, D.; Yan, W.; Chen, G.; Foster, V.; Hawdon, J. M.; Hotez, P. J. Emerging patterns of hookworm infection: influence of aging on the intensity of Necator infection in Hainan Province, People's Republic of China. Clin Infect Dis 2002, 35, 1336-44.
9. Seid, C. A.; Curti, E.; Jones, R. M.; Hudspeth, E.; Rezende, W.; Pollet, J.; Center, L.; Versteeg, L.; Pritchard, S.; Musiychuk, K.; Yusibov, V.; Hotez, P. J.; Bottazzi, M. E. Expression, purification, and characterization of the Necator americanus aspartic protease-1 (Na-APR-1 (M74)) antigen, a component of the bivalent human hookworm vaccine. Human Vaccines & Immunotherapeutics 2015, 11, 1474-1488.
10. Pearson, M. S.; Pickering, D. A.; Tribolet, L.; Cooper, L.; Mulvenna, J.; Oliveira, L. M.; Bethony, J. M.; Hotez, P. J.; Loukas, A. Neutralizing Antibodies to the Hookworm Hemoglobinase Na-APR-1: Implications for a Multivalent Vaccine against Hookworm Infection and Schistosomiasis. The Journal of Infectious Diseases 2010, 201, 1561-1569.
11. Skwarczynski, M.; Dougall, A. M.; Khoshnejad, M.; Chandrudu, S.; Pearson, M. S.; Loukas, A.; Toth, I. Peptide-based subunit vaccine against hookworm infection. PloS one 2012, 7, e46870-e46870.
12. Ahmad Fuaad, A. A. H.; Pearson, M. S.; Pickering, D. A.; Becker, L.; Zhao, G.; Loukas, A. C.; Skwarczynski, M.; Toth, I. Lipopeptide Nanoparticles: Development of Vaccines against Hookworm Parasite. ChemMedChem 2015, 10, 1647-1654.
13. Schulze, K.; Ebensen, T.; Chandrudu, S.; Skwarczynski, M.; Toth, I.; Olive, C.; Guzman, C. A. Bivalent mucosal peptide vaccines administered using the LCP carrier system stimulate protective immune responses against Streptococcus pyogenes infection. Nanomed.-Nanotechnol. Biol. Med. 2017, 13, 2463-2474.
14. Sedaghat, B.; Stephenson, R. J.; Giddam, A. K.; Eskandari, S.; Apte, S. H.; Pattinson, D. J.; Doolan, D. L.; Toth, I. Synthesis of Mannosylated Lipopeptides with Receptor Targeting Properties. Bioconjugate Chem. 2016, 27, 533-548.
15. Ahmad Fuaad, A. A.; Roubille, R.; Pearson, M. S.; Pickering, D. A.; Loukas, A. C.; Skwarczynski, M.; Toth, I. The use of a conformational cathepsin D-derived epitope for vaccine development against Schistosoma mansoni. Bioorg. Med. Chem. 2015, 23, 1307-12.
16. Skwarczynski, M.; Toth, I. Lipid-core-peptide system for self-adjuvanting synthetic vaccine delivery. In Bioconjugation Protocols, Mark, S. S., Ed. Humana Press: 2011; Vol. 751, pp 297-308.
17. Zhong, W.; Skwarczynski, M.; Toth, I. Lipid Core Peptide System for Gene, Drug, and Vaccine Delivery. Aust. J. Chem. 2009, 62, 956-967.
18. Bartlett, S.; Skwarczynski, M.; Toth, I. Lipids as activators of innate immunity in peptide vaccine delivery. 2018; Vol. 25.
19. Zhao, G.; Chandrudu, S.; Skwarczynski, M.; Toth, I. The application of self-assembled nanostructures in peptide-based subunit vaccine development. Eur. Polym. J. 2017, 93, 670-681.
20. Chan, A.; Hussein, W. M.; Ghaffar, K. A.; Marasini, N.; Mostafa, A.; Eskandari, S.; Batzloff, M. R.; Good, M. F.; Skwarczynski, M.; Toth, I. Structure-activity relationship of lipid core peptide-based Group A Streptococcus vaccine candidates. Bioorganic &Medicinal Chemistry 2016, 24, 3095-3101.
21. Zaman, M.; Chandrudu, S.; Giddam, A. K.; Reiman, J.; Skwarczynski, M.; McPhun, V.; Moyle, P. M.; Batzloff, M. R.; Good, M. F.; Toth, I. Group A Streptococcal vaccine candidate: contribution of epitope to size, antigen presenting cell interaction and immunogenicity. Nanomedicine (Lond) 2014, 9, 2613-24.
22. Skwarczynski, M.; Toth, I. Recent advances in peptide-based subunit nanovaccines. Nanomedicine (London, U. K.) 2014, 9, 2657-2669.
23. Ghaffar, K. A.; Giddam, A. K.; Zaman, M.; Skwarczynski, M.; Toth, I. Liposomes as nanovaccine delivery systems. Curr. Top. Med. Chem. 2014, 14, 1194-208.
24. Nandedkar, T. D. Nanovaccines: recent developments in vaccination. J. Biosci. 2009, 34, 995-1003.
25. des Rieux, A.; Fievez, V.; Garinot, M.; Schneider, Y. J.; Preat, V. Nanoparticles as potential oral delivery systems of proteins and vaccines: A mechanistic approach. J. Controlled Release 2006, 116, 1-27.
26. Marasini, N.; Giddam, A. K.; Ghaffar, K. A.; Batzloff, M. R.; Good, M. F.; Skwarczynski, M.; Toth, I. Multilayer engineered nanoliposomes as a novel tool for oral delivery of lipopeptide-based vaccines against group A Streptococcus. Nanomedicine-Uk 2016, 11, 1223-1236.
27. Reitermann, A.; Metzger, J.; WiesmULler, K.-H.; Jung, G.; Bessler, W. G. Lipopeptide Derivatives of Bacterial Lipoprotein Constitute Potent Immune Adjuvants Combined with or Covalently Coupled to Antigen or Hapten. In Biological Chemistry Hoppe-Seyler, 1989; Vol. 370, p 343.
28. Skwarczynski, M.; A. Kamaruzaman, K.; Srinivasan, S.; Zaman, M.; Lin, I. C.; R. Batzloff, M.; F. Good, M.; Toth, I. M-Protein-derived Conformational Peptide Epitope Vaccine Candidate against Group A Streptococcus. Current Drug Delivery 2013, 10, 39-45.
29. Skwarczynski, M.; Toth, I. Lipid-Core-Peptide System for Self-Adjuvanting Synthetic Vaccine Delivery. In Bioconjugation Protocols: Strategies and Methods, Mark, S. S., Ed. Humana Press: Totowa, N.J., 2011; pp 297-308.
30. Wang, T.; Jiang, H.; Zhao, Q.; Wang, S.; Zou, M.; Cheng, G. Enhanced mucosal and systemic immune responses obtained by porous silica nanoparticles used as an oral vaccine adjuvant: Effect of silica architecture on immunological properties. International Journal of Pharmaceutics 2012, 436, 351-358.
31. Sotillo, J.; Sanchez-Flores, A.; Cantacessi, C.; Harcus, Y.; Pickering, D.; Bouchery, T.; Camberis, M.; Tang, S. C.; Giacomin, P.; Mulvenna, J.; Mitreva, M.; Berriman, M.; LeGros, G.; Maizels, R. M.; Loukas, A. Secreted proteomes of different developmental stages of the gastrointestinal nematode Nippostrongylus brasiliensis. Mol Cell Proteomics 2014, 13, 2736-51.
32. Pearson, M. S.; Bethony, J. M.; Pickering, D. A.; de Oliveira, L. M.; Jariwala, A.; Santiago, H.; Miles, A. P.; Zhan, B.; Jiang, D.; Ranjit, N.; Mulvenna, J.; Tribolet, L.; Plieskatt, J.; Smith, T.; Bottazzi, M. E.; Jones, K.; Keegan, B.; Hotez, P. J.; Loukas, A. An enzymatically inactivated hemoglobinase from Necator americanus induces neutralizing antibodies against multiple hookworm species and protects dogs against heterologous hookworm infection. FASEB J2009, 23, 3007-19.
33. Camberis, M.; Le Gros, G.; Urban Jr., J. Animal Model of Nippostrongylus brasiliensis and Heligmosomoides polygyrus. Current Protocols in Immunology 2003, 55, 19.12.1-19.12.27.
34. Luo, Z.; Li, P.; Deng, J.; Gao, N.; Zhang, Y.; Pan, H.; Liu, L.; Wang, C.; Cai, L.; Ma, Y. Cationic polypeptide micelle-based antigen delivery system: A simple and robust adjuvant to improve vaccine efficacy. Journal of Controlled Release 2013, 170, 259-267.
35. Fuaad, A. A. H. A.; Skwarczynski, M.; Toth, I. The Use of Microwave-Assisted Solid-Phase Peptide Synthesis and Click Chemistry for the Synthesis of Vaccine Candidates Against Hookworm Infection. Methods Mol Biol 2016, 1403, 639-653.
36. Giacomin, P.; Gordon, D.; Botto, M.; Daha, M.; D Sanderson, S.; Taylor, S.; Dent, L. The role of complement in innate, adaptive and eosinophil-dependent immunity to the nematode Nippostrongylus brasiliensis. 2008; Vol. 45, p 446-55.
37. Pearson, M. S.; Bethony, J. M.; Pickering, D. A.; de Oliveira, L. M.; Jariwala, A.; Santiago, H.; Miles, A. P.; Zhan, B.; Jiang, D.; Ranjit, N.; Mulvenna, J.; Tribolet, L.; Plieskatt, J.; Smith, T.; Bottazzi, M. E.; Jones, K.; Keegan, B.; Hotez, P. J.; Loukas, A. An enzymatically inactivated hemoglobinase from Necator americanus induces neutralizing antibodies against multiple hookworm species and protects dogs against heterologous hookworm infection. FASEB journal: official publication of the Federation of American Societies for Experimental Biology 2009, 23, 3007-3019.
38. Eichenberger, R. M.; Ryan, S.; Jones, L.; Buitrago, G.; Polster, R.; Montes de Oca, M.; Zuvelek, J.; Giacomin, P. R.; Dent, L. A.; Engwerda, C. R.; Field, M. A.; Sotillo, J.; Loukas, A. Hookworm Secreted Extracellular Vesicles Interact With Host Cells and Prevent Inducible Colitis in Mice. Frontiers in immunology 2018, 9, 850-850.

### REFERENCES—EXAMPLE 4

1. Skwarczynski, M.; Zhao, G.; Boer, J. C.; Ozberk, V.; Azuar, A.; Cruz, J. G.; Giddam, A. K.; Khalil, Z. G.; Pandey, M.; Shibu, M. A.; Hussein, W. M.; Nevagi, R. J.; Batzloff, M. R.; Wells, J. W.; Capon, R. J.; Plebanski, M.; Good, M. F.; Toth I. Poly(amino acids) as a Potent Self-adjuvanting Delivery System for Peptide-based Nanovaccines Science Advances 2020, 6, eaax2285.
2. Ahmad Fuaad, A. A. H.; Skwarczynski, M.; Toth, I. The Use of Microwave-Assisted Solid-Phase Peptide Synthesis and Click Chemistry for the Synthesis of Vaccine Candidates Against Hookworm Infection. In Vaccine Design: Methods and Protocols: Volume 1: Vaccines for Human Diseases, Thomas, S., Ed. Springer New York: New York, N.Y., 2016; pp 639-653.
3. Skwarczynski, M.; Zaman, M.; Toth, I. Lipo-Peptides/Saccharides for Peptide Vaccine Delivery—Chapter 78. Elsevier Inc.: 2013; p 571-579.
4. Jadhav, K. B., Woolcock, K. J., & Muttenthaler, M. (2020). Anhydrous Hydrogen Fluoride Cleavage in Boc Solid Phase Peptide Synthesis. Methods in molecular biology (Clifton, N.J.), 103, 41--57. https://doi.org/10.1007/978-1-0716-0227-0_4.
5. Greenfield, N. J. Using circular dichroism spectra to estimate protein secondary structure. Nat. Protoc. 2006, 1, 2876-2890.

### REFERENCES—EXAMPLE 5

1. Vaccination. In Primer to the Immune Response (Second Edition), Mak, T. W.; Saunders, M. E.; Jett, B. D., Eds. Academic Cell: Boston, 2014; pp 333-375.
2. Chang, K. P. Vaccination for disease prevention and control: The necessity of renewed emphasis and new approaches. J Immunol Immunotech 2014, 1, 10.17653/2374-9105.SSe0001.
3. Skwarczynski, M.; Toth, I. Peptide-based synthetic vaccines. Chem. Sci. 2016, 7, 842-854.
4. Malonis, R. J.; Lai, J. R.; Vergnolle, O. Peptide-based vaccines: Current progress and future challenges. Chem. Rev. 2020, 120, 3210-3229.
5. Christensen, D. Vaccine adjuvants: Why and how. Human vaccines & immunotherapeutics 2016, 12, 2709-2711.
6. Azmi, F.; Ahmad Fuaad, A. A.; Skwarczynski, M.; Toth, I. Recent progress in adjuvant discovery for peptide-based subunit vaccines. Hum Vaccin Immunother 2014, 10, 778-796.
7. Shi, S.; Zhu, H.; Xia, X.; Liang, Z.; Ma, X.; Sun, B. Vaccine adjuvants: Understanding the structure and mechanism of adjuvanticity. Vaccine 2019, 37, 3167-3178.
8. Nevagi, R. J.; Toth, I.; Skwarczynski, M. Peptide-based Vaccines. In Peptide Applications in Biomedicine, Biotechnology and Bioengineering, Koutsopoulos, S., Ed. Woodhead Publishing: Oxford, United Kingdom, 2018; pp 327-358.
9. Skwarczynski, M.; Toth, I. Recent advances in peptide-based subunit nanovaccines. Nanomedicine (London, U. K.) 2014, 9, 2657-2669.
10. Sun, B.; Xia, T. Nanomaterial-based vaccine adjuvants. J Mater Chem B 2016, 4, 5496-5509.
11. Lebre, F.; Hearnden, C. H.; Lavelle, E. C. Modulation of Immune Responses by Particulate Materials. Advanced Materials 2016, 28, 5525-5541.
12. Zhao, G.; Chandrudu, S.; Skwarczynski, M.; Toth, I. The application of self-assembled nanostructures in peptide-based subunit vaccine development. Eur. Polym. J. 2017, 93, 670-681.
13. Negahdaripour, M.; Golkar, N.; Hajighahramani, N.; Kianpour, S.; Nezafat, N.; Ghasemi, Y. Harnessing self-assembled peptide nanoparticles in epitope vaccine design. Biotechnol. Adv. 2017, 35, 575-596.
14. Black, M.; Trent, A.; Kostenko, Y.; Lee, J. S.; Olive, C.; Tirrell, M. Self-Assembled Peptide Amphiphile Micelles Containing a Cytotoxic T-Cell Epitope Promote a Protective Immune Response In Vivo. Advanced Materials 2012, 24, 3845-3849.
15. Skwarczynski, M.; Zaman, M.; Urbani, C. N.; Lin, I. C.; Jia, Z.; Batzloff, M. R.; Good, M. F.; Monteiro, M. J.; Toth, I. Polyacrylate dendrimer nanoparticles: a self-adjuvanting vaccine delivery system. Angew. Chem. Int. Ed. Engl. 2010, 49, 5742-5745.
16. Zaman, M.; Abdel-Aal, A.-B. M.; Fujita, Y.; Phillipps, K. S. M.; Batzloff, M. R.; Good, M. F.; Toth, I. Immunological Evaluation of Lipopeptide Group A Streptococcus (GAS) Vaccine: Structure-Activity Relationship. PLoS One 2012, 7, e30146.
17. Nevagi, R. J.; Skwarczynski, M.; Toth, I. Polymers for subunit vaccine delivery. Eur. Polym. J. 2019, 114, 397-410.
18. Bartlett, S.; Skwarczynski, M.; Toth, I. Lipids as Activators of Innate Immunity in Peptide Vaccine Delivery. Curr. Med. Chem. 2020, 27, 2887-2901.
19. Abdel-Aal, A. B. M.; Batzloff, M. R.; Fujita, Y.; Barozzi, N.; Faria, A.; Simerska, P.; Moyle, P. M.; Good, M. F.; Toth, I. Structure-activity relationship of a series of synthetic lipopeptide self-adjuvanting group A streptococcal vaccine candidates. J. Med. Chem. 2008, 51, 167-172.
20. Faruck, M. O.; Zhao, L.; Hussein, W. M.; Khalil, Z. G.; Capon, R. J.; Skwarczynski, M.; Toth, I. Polyacrylate-Peptide Antigen Conjugate as a Single-Dose Oral Vaccine against Group A Streptococcus. Vaccines 2020, 8, 23.
21. Chandrudu, S.; Bartlett, S.; Khalil, Z. G.; Jia, Z.; Hussein, W. M.; Capon, R. J.; Batzloff, M. R.; Good, M. F.; Monteiro, M. J.; Skwarczynski, M.; Toth, I. Linear and branched polyacrylates as a delivery platform for peptide-based vaccines. Ther Deliv 2016, 7, 601-609.
22. Liu, T. Y.; Hussein, W. M.; Giddam, A. K.; Jia, Z.; Reiman, J. M.; Zaman, M.; McMillan, N. A.; Good, M. F.; Monteiro, M. J.; Toth, I.; Skwarczynski, M. Polyacrylate-based delivery system for self-adjuvanting anticancer peptide vaccine. J. Med. Chem. 2015, 58, 888-896.
23. Ahmad Fuaad, A. A.; Jia, Z.; Zaman, M.; Hartas, J.; Ziora, Z. M.; Lin, I. C.; Moyle, P. M.; Batzloff, M. R.; Good, M. F.; Monteiro, M. J.; Skwarczynski, M.; Toth, I. Polymer-peptide hybrids as a highly immunogenic single-dose nanovaccine. Nanomedicine (Lond) 2014, 9, 35-43
24. Liu, T. Y.; Hussein, W. M.; Jia, Z.; Ziora, Z. M.; McMillan, N. A.; Monteiro, M. J.; Toth, I.; Skwarczynski, M. Self-adjuvanting polymer-peptide conjugates as therapeutic vaccine candidates against cervical cancer. Biomacromolecules 2013, 14, 2798-2806.
25. Skwarczynski, M.; Zhao, G.; Boer, J. C.; Ozberk, V.; Azuar, A.; Cruz, J. G.; Giddam, A. K.; Khalil, Z. G.; Pandey, M.; Shibu, M. A.; Hussein, W. M.; Nevagi, R.; Batzloff, M. R.; Wells, J. W.; Capon, R. J.; Plebanski, M.; Good, M. F.; Toth, I. Poly(amino acids) as a potent self-adjuvanting delivery system for peptide-based nanovaccines. Sci Adv 2020, 6, eaax2285.
26. Nevagi, R. J.; Dai, W.; Khalil, Z. G.; Hussein, W. M.; Capon, R. J.; Skwarczynski, M.; Toth, I. Structure-activity relationship of group A Streptococcus lipopeptide vaccine candidates in trimethyl chitosan-based self-adjuvanting delivery system. Eur. J. Med. Chem. 2019, 179, 100-108.
27. Chan, A.; Hussein, W. M.; Ghaffar, K. A.; Marasini, N.; Mostafa, A.; Eskandari, S.; Batzloff, M. R.; Good, M. F.; Skwarczynski, M.; Toth, I. Structure-activity relationship of lipid core peptide-based Group A Streptococcus vaccine candidates. Bioorg. Med. Chem. 2016, 24, 3095-3101.
28. Zeng, W.; Ghosh, S.; Lau, Y. F.; Brown, L. E.; Jackson, D. C. Highly immunogenic and totally synthetic lipopeptides as self-adjuvanting immunocontraceptive vaccines. J. Immunol. 2002, 169, 4905-4912.
29. Zaman, M.; Abdel-Aal, A.-B. M.; Fujita, Y.; Ziora, Z. M.; Batzloff, M. R.; Good, M. F.; Toth, I. Structure-activity relationship for the development of a self-adjuvanting mucosally active lipopeptide vaccine against Streptococcus pyogenes. J. Med. Chem. 2012, 55, 8515-8523.
30. Eskandari, S.; Pattinson, D. J.; Stephenson, R. J.; Groves, P. L.; Apte, S. H.; Sedaghat, B.; Chandurudu, S.; Doolan, D. L.; Toth, I. Influence of physicochemical properties of lipopeptide adjuvants on the immune response: A rationale for engineering a potent vaccine. Chem-Eur J 2018, 24, 9892-9902.
31. Hayman, W. A.; Brandt, E. R.; Relf, W. A.; Cooper, J.; Saul, A.; Good, M. F. Mapping the minimal murine T cell and B cell epitopes within a peptide vaccine candidate from the conserved region of the M protein of group A Streptococcus. Int. Immunol. 1997, 9, 1723-1733.
32. Alexander, J.; Del Guercio, M.-F.; Maewal, A.; Fikes, J.; Chesnut, R. W.; Sette, A.; Qiao, L.; Paulson, J.; DeFrees, S.; Bundle, D. R. Linear PADRE T helper epitope and carbohydrate B cell epitope conjugates induce specific high titer IgG antibody responses. J Immunol 2000, 164, 1625-1633.
33. Sekuloski, S.; Batzloff, M. R.; Griffin, P.; Parsonage, W.; Elliott, S.; Hartas, J.; O'Rourke, P.; Marquart, L.; Pandey, M.; Rubin, F. A.; Carapetis, J.; McCarthy, J.; Good, M. F. Evaluation of safety and immunogenicity of a group A Streptococcus vaccine candidate (MJ8VAX) in a randomized clinical trial. PLoS One 2018, 13, e0198658.
34. La Rosa, C.; Longmate, J.; Lacey, S. F.; Kaltcheva, T.; Sharan, R.; Marsano, D.; Kwon, P.; Drake, J.; Williams, B.; Denison, S.; Broyer, S.; Couture, L.; Nakamura, R.; Kelsey, M. I.; Krieg, A. M.; Diamond, D. J.; Zaia, J. A. Clinical evaluation of safety and immunogenicity of PADRE-cytomegalovirus (CMV) and tetanus-CMV fusion peptide vaccines with or without PF03512676 adjuvant. J. Infect. Dis. 2012, 205, 1294-1304
35. Malkov, S. N.; Zivkovic, M. V.; Beljanski, M. V.; Hall, M. B.; Zaric, S. D. A reexamination of the propensities of amino acids towards a particular secondary structure: classification of amino acids based on their chemical structure. J. Mol. Model. 2008, 14, 769-775.
36. Azuar, A.; Jin, W.; Mukaida, S.; Hussein, W.; Toth, I.; Skwarczynski, M. Recent advances in the development of peptide vaccines and their delivery systems against Group A Streptococcus. Vaccines 2019, 7, 10.3390/vaccines7030058.
37. Reed, S. G.; Orr, M. T.; Fox, C. B. Key roles of adjuvants in modern vaccines. Nat. Med. 2013, 19, 1597-1608.
38. Colaco, M.; Park, J.; Blanch, H. The kinetics of aggregation of poly-glutamic acid based polypeptides. Biophys Chem 2008, 136, 74-86.
39. Nevagi, R. J.; Khalil, Z. G.; Hussein, W. M.; Powell, J.; Batzloff, M. R.; Capon, R. J.; Good, M. F.; Skwarczynski, M.; Toth, I. Polyglutamic acid-trimethyl chitosan-based intranasal peptide nano-vaccine induces potent immune responses against group A Streptococcus. Acta Biomater. 2018, 80, 278-287.
40. Lilliu, E.; Villeri, V.; Pelassa, I.; Cesano, F.; Scarano, D.; Fiumara, F. Polyserine repeats promote coiled coil-mediated fibril formation and length-dependent protein aggregation. J. Struct. Biol. 2018, 204, 572-584.
41. Ohnishi, S.; Kamikubo, H.; Onitsuka, M.; Kataoka, M.; Shortle, D. Conformational preference of polyglycine in solution to elongated structure. J. Am. Chem. Soc. 2006, 128, 16338-16344.
42. Nordstrom, T.; Pandey, M.; Calcutt, A.; Powell, J.; Phillips, Z. N.; Yeung, G.; Giddam, A. K.; Shi, Y.; Haselhorst, T.; von Itzstein, M.; Batzloff, M. R.; Good, M. F. Enhancing vaccine efficacy by engineering a complex synthetic peptide to become a super immunogen. J. Immunol. 2017, 199, 2794-2802.
43. Doruker, P.; Bahar, I. Role of water on unfolding kinetics of helical peptides studied by molecular dynamics simulations. Biophysical journal 1997, 72, 2445-2456.
44. Marqusee, S.; Robbins, V. H.; Baldwin, R. L. Unusually stable helix formation in short alanine-based peptides. Proc. Natl. Acad. Sci. U.S.A 1989, 86, 5286-5290.
45. Forood, B.; Perez-Paya, E.; Houghten, R. A.; Blondelle, S. E. Formation of an extremely stable polyalanine beta-sheet macromolecule. Biochem. Biophys. Res. Commun. 1995, 211, 7-13.
46. Blondelle, S. E.; Forood, B.; Houghten, R. A.; PerezPaya, E. Polyalanine-based peptides as models for self-associated beta-pleated-sheet complexes. Biochemistry 1997, 36, 8393-8400.
47. Rucker, A. L.; Creamer, T. P. Polyproline II helical structure in protein unfolded states: lysine peptides revisited. Protein Sci. 2002, 11, 980-985.
48. Rivera-Hernandez, T.; Rhyme, M. S.; Cork, A. J.; Jones, S.; Segui-Perez, C.; Brunner, L.; Richter, J.; Petrovsky, N.; Lawrenz, M.; Goldblatt, D.; Collin, N.; Walker, M. J. Vaccine-induced Th1-type response protects against invasive Group A Streptococcus infection in the absence of opsonizing antibodies. Mbio 2020, 11, e00122-20.
49. Ahmad Fuaad, A. A. H.; Skwarczynski, M.; Toth, I. The use of microwave-assisted solid-phase peptide synthesis and click chemistry for the synthesis of vaccine candidates against hookworm infection. In Methods in molecular biology (Clifton, N.J.), Thomas, S., Ed. Springer Science+Business Media: New York, 2016; Vol. 1403, pp 639-653.
50. Greenfield, N. J. Using circular dichroism spectra to estimate protein secondary structure. Nat. Protoc. 2006, 1, 2876-2890.

### REFERENCES—EXAMPLE 6

1. Organization, W. H., Coronavirus disease 2019 (COVID-19): situation report, 72. 2020.
2. Nicola, M., et al., The socio-economic implications of the coronavirus pandemic (COVID-19): A review. Int J Surg, 2020. 78: p. 185-193.
3. Tu, Y.-F., et al., A Review of SARS-CoV-2 and the Ongoing Clinical Trials. International journal of molecular sciences, 2020. 21(7): p. 2657.
4. Anderson, E. J., et al., Safety and Immunogenicity of SARS-CoV-2 mRNA-1273 Vaccine in Older Adults. N Engl J Med, 2020.
5. Folegatti, P. M., et al., Safety and immunogenicity of the ChAdOx1 nCoV-19 vaccine against SARS-CoV-2: a preliminary report of a phase 1/2, single-blind, randomised controlled trial. The Lancet, 2020. 396(10249): p. 467-478.
6. Mulligan, M. J., et al., Phase I/II study of COVID-19 RNA vaccine BNT162b1 in adults. Nature, 2020. 586(7830): p. 589-593.
7. Tang, S., et al., Aerosol transmission of SARS-CoV-2? Evidence, prevention and control. Environment international, 2020. 144: p. 106039-106039.
8. Wrapp, D., et al., Cryo-EM structure of the 2019-nCoV spike in the prefusion conformation. Science, 2020. 367(6483): p. 1260-1263.
9. Naqvi, A. A. T., et al., Insights into SARS-CoV-2 genome, structure, evolution, pathogenesis and therapies: Structural genomics approach. Biochimica et biophysica acta. Molecular basis of disease, 2020. 1866(10): p. 165878-165878.
10. Piccoli, L., et al., Mapping Neutralizing and Immunodominant Sites on the SARS-CoV-2 Spike Receptor-Binding Domain by Structure-Guided High-Resolution Serology. Cell, 2020.
11. Tian, X., et al., Potent binding of 2019 novel coronavirus spike protein by a SARS coronavirus-specific human monoclonal antibody. Emerg Microbes Infect, 2020. 9(1): p. 382-385.
12. Li, C. K., et al., T cell responses to whole SARS coronavirus in humans. J Immunol, 2008. 181(8): p. 5490-500.
13. Chi, X., et al., A neutralizing human antibody binds to the N-terminal domain of the Spike protein of SARS-CoV-2. Science, 2020. 369(6504): p. 650-655.
14. Thevarajan, I., et al., Breadth of concomitant immune responses prior to patient recovery: a case report of non-severe COVID-19. Nat Med, 2020. 26(4): p. 453-455.
15. Zhang, Y., et al., Inflammatory Response Cells During Acute Respiratory Distress Syndrome in Patients With Coronavirus Disease 2019 (COVID-19). Ann Intern Med, 2020.
16. Zhou, F., et al., Clinical course and risk factors for mortality of adult inpatients with COVID-19 in Wuhan, China: a retrospective cohort study. Lancet, 2020. 395(10229): p. 1054-1062.
17. Tay, M. Z., et al., The trinity of COVID-19: immunity, inflammation and intervention. Nat Rev Immunol, 2020. 20(6): p. 363-374.
18. Eroshenko, N., et al., Implications of antibody-dependent enhancement of infection for SARS-CoV-2 countermeasures. Nature Biotechnology, 2020. 38(7): p. 789-791.
19. Wang, Q., et al., Immunodominant SARS Coronavirus Epitopes in Humans Elicited both Enhancing and Neutralizing Effects on Infection in Non-human Primates. ACS Infect Dis, 2016. 2(5): p. 361-76.
20. Sekimukai, H., et al., Gold nanoparticle-adjuvanted S protein induces a strong antigen-specific IgG response against severe acute respiratory syndrome-related coronavirus infection, but fails to induce protective antibodies and limit eosinophilic infiltration in lungs. Microbiol Immunol, 2020. 64(1): p. 33-51.
21. Tseng, C. T., et al., Immunization with SARS coronavirus vaccines leads to pulmonary immunopathology on challenge with the SARS virus. PLoS One, 2012. 7(4): p. e35421.
22. Skwarczynski, M. and I. Toth, Peptide-based synthetic vaccines. Chem. Sci., 2016. 7(2): p. 842-854.
23. Berry, J. D., et al., Neutralizing epitopes of the SARS-CoV S-protein cluster independent of repertoire, antigen structure or mAb technology. MAbs, 2010. 2(1): p. 53-66.
24. Channappanavar, R., et al., Virus-specific memory CD8 T cells provide substantial protection from lethal severe acute respiratory syndrome coronavirus infection. J Virol, 2014. 88(19): p. 11034-44.
25. Zhang, B.-z., et al., Mapping the Immunodominance Landscape of SARS-CoV-2 Spike Protein for the Design of Vaccines against COVID-19. 2020: p. 2020.04.23.056853.
26. Zhang, B.-z., et al., Mining of epitopes on spike protein of SARS-CoV-2 from COVID-19 patients. Cell Research, 2020. 30(8): p. 702-704.
27. Dai, C., et al., Application of Fmoc-SPPS, Thiol-Maleimide Conjugation, and Copper(I)-Catalyzed Alkyne-Azide Cycloaddition “Click” Reaction in the Synthesis of a Complex Peptide-Based Vaccine Candidate Against Group A Streptococcus, in Methods in molecular biology (Clifton, N.J.). 2020. p. 13-27.
28. Byrnes, J. R., et al., A SARS-CoV-2 serological assay to determine the presence of blocking antibodies that compete for human ACE2 binding. medRxiv: the preprint server for health sciences, 2020: p. 2020.05.27.20114652.
29. He, Y., et al., Receptor-binding domain of severe acute respiratory syndrome coronavirus spike protein contains multiple conformation-dependent epitopes that induce highly potent neutralizing antibodies. J Immunol, 2005. 174(8): p. 4908-15.
30. Alexander, J., et al., Linear PADRE T helper epitope and carbohydrate B cell epitope conjugates induce specific high titer IgG antibody responses. Journal of Immunology, 2000. 164(3): p. 1625-1633.

1. An immunogenic agent suitable for administration to a subject, said immunogenic agent comprising a hydrophobic peptide covalently coupled or conjugated to at least one antigenic molecule.

2. An immunogenic agent having the structure: [Antigenic molecule]ₙ-[Carrier/Linker]ₘ-[Hydrophobic peptide],
wherein: n is 1 or a higher number;
m is 0 or a higher number; and
“-” represents a covalent coupling or conjugation.

3. A method of producing an immunogenic agent, comprising the step of covalently coupling a hydrophobic peptide to at least one antigenic molecule to thereby produce the immunogenic agent.

4. An immunogenic agent when produced by the method according to claim 3.

5. An immunogenic complex comprising a plurality of immunogenic agents according to any one of claims 1, 2 and 4, wherein a plurality of hydrophobic peptides interact in the immunogenic complex.

6. A method of producing an immunogenic complex comprising the step of combining a plurality of immunogenic agents of any one of claims 1, 2 and 4, whereby the plurality of immunogenic agents self-assemble into the immunogenic complex.

7. An immunogenic complex when produced by the method of claim 6.

8. The immunogenic agent of any one of claims 1, 2 and 4, the immunogenic complex of claim 5 or claim 7, or the method of claim 3 or claim 6, wherein the hydrophobic peptide comprises or consists of natural amino acids.

9. The immunogenic agent of any one of claims 1, 2, 4 and 8, the immunogenic complex of any one of claims 5, 7 and 8, or the method of any one of claims 3, 6 and 8, wherein the hydrophobic peptide comprises or consists of anywhere between about 2 and about 50 amino acids.

10. The immunogenic agent of any one of claims 1, 2, 4, 8 and 9, the immunogenic complex of any one of claims 5 and 7-9, or the method of any one of claims 3, 6, 8 and 9, wherein the hydrophobic amino acids are preferably selected from the group consisting of: glycine, proline, valine, alanine, phenylalanine, leucine, polyglycine, polyproline, polyvaline, polyalanine, polyphenylalanine and polyleucine.

11. The immunogenic agent of any one of claims 1, 2, 4 and 8-10, the immunogenic complex of any one of claims 5 and 7-10, or the method of any one of claims 3, 6 and 8-10, wherein the hydrophobic peptide enables anchoring of the immunogenic agent to a liposomal membrane or other lipophilic surface such as a micelle or nanoparticle.

12. The immunogenic agent of any one of claims 1, 2, 4 and 8-11, the immunogenic complex of any one of claims 5 and 7-11, or the method of any one of claims 3, 6 and 8-11, wherein:
the conformation of the at least one antigenic molecule is controlled by changing the amino acid sequence and/or length of the hydrophobic peptide; and/or
the solubility of the immunogenic agent is controlled by changing the amino acid sequence and/or length of the hydrophobic peptide.

13. The immunogenic agent of any one of claims 1, 2, 4 and 8-12, the immunogenic complex of any one of claims 5 and 7-12, or the method of any one of claims 3, 6 and 8-12, wherein the immunogenic agent is linear, branched or dendritic in structure.

14. The immunogenic agent of any one of claims 1, 2, 4 and 8-13, the immunogenic complex of any one of claims 5 and 7-13, or the method of any one of claims 3, 6 and 8-13, wherein the at least one antigenic molecule comprises, consists essentially of, or consists of a peptide, a protein or carbohydrate, or a fragment or a derivative thereof.

15. The immunogenic agent of any one of claims 1, 2, 4 and 8-14, the immunogenic complex of any one of claims 5 and 7-14, or the method of any one of claims 3, 6 and 8-14, wherein the at least one antigenic molecule or fragment or derivative thereof, is of a pathogen.

16. The immunogenic agent of any one of claims 1, 2, 4 and 8-15, the immunogenic complex of any one of claims 5 and 7-15, or the method of any one of claims 3, 6 and 8-15, wherein the at least one antigenic molecule and the hydrophobic peptide are situated at opposed termini of the immunogenic agent.

17. The immunogenic agent of any one of claims 1, 2, 4 and 8-16, the immunogenic complex of any one of claims 5 and 7-16, or the method of any one of claims 3, 6 and 8-16, wherein when the immunogenic agent comprises a plurality of antigenic molecules, a carrier or linker is utilised to couple or connect the antigenic molecules together in a contiguous, branched or dendritic form or manner.

18. The immunogenic agent of any one of claims 1, 2, 4 and 8-17, the immunogenic complex of any one of claims 5 and 7-17, or the method of any one of claims 3, 6 and 8-17, wherein:
at least one carrier or linker balances the hydrophobic:hydrophilic ratio of the immunogenic agent to assist with proper conformation of the immunogenic agent, and/or solubility of the immunogenic agent; and/or
at least one carrier or linker comprises at least one amino acid that is capable of forming a branched chain, for attachment to the hydrophobic peptide.

19. The immunogenic agent of any one of claims 1, 2, 4 and 8-18, the immunogenic complex of any one of claims 5 and 7-18, or the method of any one of claims 3, 6 and 8-18, wherein:
the at least one antigenic molecule consists of or comprises the SARS-CoV spike protein epitope AIHADQLTPTWRVYSTG (S₆₂₃₋₆₃₉) (SEQ ID NO:15);
the at least one antigenic molecule consists of or comprises the SARS-CoV spike protein epitope STEIYQAGSTPCNGVEGFNCYFPLQSYGFQPTNGVGYQPY (S_{469_508}) (SEQ ID NO:16);
the at least one antigenic molecule consists of or comprises the SARS-CoV spike protein epitope VGGNYNYLYRLFRKSNLKPFERDISTEIYQAGSTPCNGV (S_{445_483}) (SEQ ID NO:17);
the at least one antigenic molecule consists of or comprises the SARS-CoV spike protein epitope FLPFQQFGRDIADT (S₅₅₉₋₅₇₂) (SEQ ID NO:18); and/or
the at least one antigenic molecule consists of or comprises the SARS-CoV spike protein epitope SVLYNSASFSTFKCYGVSPTKLNDLCFTNV (S_{366_395}) (SEQ ID NO:19).

20. A composition comprising at least one said immunogenic agent of any one of claims 1, 2, 4 and 8-19, and/or at least one said immunogenic complex of any one of claims 5 and 7-19, and at least one acceptable carrier, diluent or excipient.

21. The composition of claim 20, further comprising:
liposomes, micelles or nanoparticles; or
liposomes, micelles or nanoparticles, and a mannose targeting moiety.

22. A method of eliciting an immune response in a subject, said method comprising the step of administering to the subject at least one said immunogenic agent of any one of claims 1, 2, 4 and 8-19, at least one said immunogenic complex of any one of claims 5 and 7-19, or at least one said composition of claim 20 or claim 21, to thereby elicit an immune response in the subject.

23. A method of immunizing a subject, said method comprising the step of administering to the subject at least one said immunogenic agent of any one of claims 1, 2, 4 and 8-19, at least one said immunogenic complex of any one of claims 5 and 7-19, or at least one said composition of claim 20 or claim 21, to thereby immunize the subject.

24. A method of treating or preventing a disease, disorder or condition in a subject, said method comprising the step of administering to the subject at least one said immunogenic agent of any one of claims 1, 2, 4 and 8-19, at least one said immunogenic complex of any one of claims 5 and 7-19, or at least one said composition of claim 20 or claim 21.

25. The method of any one of claims 22-24, wherein:
the animal is a mammal, bird, pig or human; and/or
the method does not require co-administration of an adjuvant or other general immune-stimulant.
